(19) United States
(12) Patent Application Publication
(10) Pub. No.: US 2005/0241012 A1
(43) Pub. Date: Oct. 27, 2005

(54) Multigene profiling in the kidney to tailor drug therapy

(76) Inventors: Sanjay Kumar Nigam, Del Mar, CA (US); Satish A. Eraly, San Diego, CA (US)

Correspondence Address: BUCHANAN INGERSOLL LLP;(INCLUDING BURNS, DOANE, SWECKER & MATHIS), 12230 EL CAMINO REAL, SUITE 300, SAN DIEGO, CA, 92130, US

(21) Appl. No.: 10/968,848

(22) Filed: Oct. 18, 2004

Related U.S. Application Data
(60) Provisional application No. 60/512,550, filed on Oct. 17, 2003.

(51) Int. Cl. A01K 67/027

(52) U.S. Cl. 800/18

(57) ABSTRACT

Provided are compositions, transgenic animals and methods for screening and analyzing drugs for toxicity and clearance. Such methods include creating a transgenic animal that lacks expression of two or more slc22 family member—organic ion transporters. Further disclosed are methods useful in determining a subjects sensitivity and a drugs efficacy based upon single nucleotide length polymorphisms in organic ion transport genes.

### CROSS REFERENCE TO RELATED APPLICATIONS

This application claims priority under 35 U.S.C. §119 from Provisional Application Ser. No. 60/512,550, filed Oct. 17, 2003, the disclosure of which is incorporated herein by reference.

### STATEMENT REGARDING FEDERALLY SPONSORED RESEARCH

The invention was funded in part by Grant No. R01-HD40011 awarded by NICHD. The government may have certain rights in the invention.

### TECHNICAL FIELD

This invention relates to transgenic organisms, more particularly related to knockout organisms lacking one or more organic anion transporters (OATs), methods of identifying polymorphisms associated with specific OATs and methods of tailoring drug therapy.

### BACKGROUND

Multiple drug transporters are present in various organs of the body including the kidney, the intestinal epithelium, the brain microvessel endothelium, and the liver. Humans are highly variable in their ability to metabolize and excrete drugs. This variability underlies a great deal of morbidity and mortality as standardized doses of medications can produce drug levels that range from sub-therapeutic to toxic. Organic anion and cation transporters (OATs and OCTs, respectively) a family of transmembrane proteins largely expressed in excretory organs such as kidney and liver are a major component of the human xenobiotic excretion machinery. These proteins interact with many commonly used drugs including antibiotics, anti-hypertensives, and anti-inflammatories among others. As such it is probable that variations in either the coding or regulatory sequences of OAT and OCT genes contribute significantly to differences in drug-handling capability. Delineation of the genomic structure and critical cis-regulatory elements of these genes will be an essential step in correlating such variations with clinical phenotypes.

### SUMMARY

The organic anion transport system of the renal proximal tubule is responsible for the excretion of many pharmaceuticals of great clinical significance; these include numerous antibiotics, antivirals, antihypertensives, and anti-inflammatories. This transport system proceeds in two steps: basolateral uptake followed by apical secretion. Each step appears to be mediated by a pair of functionally redundant organic anion transporter (OAT) proteins. Basolateral entry is due to, for example, OAT1 and OAT3, while apical exit is due to, for example, OAT4 and RST.

Because of the necessity of anionic and cationic transport in tissue metabolism processes, OATs and OCTs have been implicated in numerous drug interactions and nephrotoxic drug reactions. The study of OAT and OCTs, including their genetics and regulation, is expected to be crucial to renal pharmacology and pharmacogenetics.

The invention provides OAT double knockout non-human transgenic animals. Knocking out either the apical or basolateral OAT pair, or one apical and one basolateral OAT in mice (e.g., “double” knockouts) would create highly sensitive animal models for the detection of drug toxicity. Knockout of the apical pair (e.g., OAT4/RST) would result in proximal tubular accumulation of substrates (due to the “unopposed” action of the basolateral OATs). Thus, OAT4/RST double knockout animals, for example, would be highly sensitive to nephrotoxic OAT substrates, and could accordingly be used to screen for such toxicity. Conversely, knockout of the basolateral pair (e.g., OAT1/OAT3) would lead to delayed clearance of substrates. Thus, OAT1/OAT3 double knockout animals, for example, would represent a sensitive model system in which to screen for extra-renal/systemic toxicity.

In addition, polymorphisms in human OAT genes that are likely to affect function (based on their effects on transport activity in vitro) can be introduced into mice (by homologous recombination) to create “humanized” mice representing different OAT variants. These mice would represent important models in which to test the impact of various human OAT polymorphisms on drug-handling and toxicity.

The invention also provides methods of determining the susceptibility of a subject to a particular drug based upon a polymorphism. For example, following characterization of the functional consequences of various OAT polymorphisms, an “OAT genotype” of a subject (based upon the particular polymorphisms that are present) could be determined. Techniques for identifying polymorphisms are known including the use of microarrays. Identification of a particular polymorphism is useful in order to guide drug therapy. For example, if a particular polymorphism were known to be associated with a predisposition to toxicity from a certain drug, that drug would be avoided in subjects carrying that polymorphism. The complement of polymorphisms present in an individual subject could be readily determined through such a screening method.

Using the methods and compositions of the invention it will be possible to predict potential OAT substrates and inhibitors based on molecular modeling of transporters. From molecular structural modeling information, it will be possible to predict which novel drugs are likely to pass through these transporters and design inhibitors.

The invention also provides an OAT6 polypeptide, polynucleotide, vectors, host cells and methods of use.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic depicting the Basolateral entry of organic anions(OAs). Basolateral OA entry occurs in exchange for intracellular dicarboxylates (DCs). The latter are maintained at high concentrations through the action of a sodium-dicarboxylate cobrane transporter, which in turn is driven by the sodium gradient established by the sodium/potassium ATPase. The mechanism of apical exit of organic anions is sodium-independent and may involve anion exchange.

FIG. 2 shows the sequences of organic anion transporters (OATs)1-5, UST1, and UST3, as well as sequences of organic cation transporters (OCTs), novel organic cation transporters (OCTNs), and fly-like putative transporters (Flipts) were aligned (ClustalX), and the alignment output used to generate a dendrogram. Paired genes are enclosed in ellipses, and the text boxes next to the pairs indicate (where known) the tissue distribution, tubular localization, membrane localization, and transport mechanism of the pair members (reading from top to bottom).

FIGS. 3A and B shows is a diagram depicting the role of transporters in the kidney. (A) Basolateral organic anion transporters (OATS) in the renal proximal tubule mediate uptake of potentially harmful substrates (heavy black arrow), resulting in nephrotoxicity. (B) Competition from other OAT substrates (gray arrow) results in decreased uptake of toxins, and thus decreased nephrotoxicity. However, such competition might also result in delayed clearance, and therefore increased extra-renal toxicity.

FIG. 4(A) Knockout of apical organic anion transporters (OATS) can result in increased nephrotoxicity due to unopposed transport of potential nephrotoxins into the proximal tubular cell by basolateral OATS.

FIG. 4(B) Knockout of basolateral OATS can lead to increased extrarenal toxicity due to delayed clearance.

FIG. 5 Exon/intron structures of murine OAT1 and 3. Exons (gray rectangles) and introns (the lines joining them) are set to the same scale. The sizes of exons in base pairs (bp) and the sizes of introns in kilobases (kb) are indicated below the corresponding exons and intron numbers, respectively. The overall sizes of the two genes, the relative orientation, and the intergenic distance between them are indicated below the structures.

FIG. 6. Phylogenetic footprints (PFs) in the 5′ flanking regions of OAT1 (equivalent to the OAT1-3 intergenic region) and OAT3. The entire intergenic sequence between murine OAT1 and 3 (7.5 kb) was compared by pair-wise BLAST to the corresponding sequence from the human genome, delineating three PFs (PFI 1-3) in the OAT1 5′ flanking region. Similarly, 10 kb of 5′ flanking (upstream) sequence form the murine and human OAT3 was compared to delineate five PFs (PFu 1-5) in the OAT3 5′ flanking region. Footprints are numbered in decreasing order of significance (please refer to Table 1 for the properties). The locations of the footprints are depicted schematically at the top of the figure.

FIG. 7A and B shows conserved transcription factor binding sites in the Pfs upstream of OAT3 (PFu 1-5; A) and OAT1 (Pfi 1-3; B). The sequences of the indicated PFs were examined for the presence of matches to binding-site matrices from the TransFac database. Matches present in both the mouse and human sequences are boxed, with matches to factors implicated in kidney development highlighted in gray. Only the sequences of the sense (“top”) strands are depicted in the alignments, with the upper sequence in each alignment being from mouse and the lower sequence from human. Matches to the sense strand are indicated with “+” and matches to the complementary antisense strand are indicated with “−”. Sequence numberings are relative to the transcription start site of the corresponding mouse gene.

FIGS. 8A and B show the phylogenic relationships and expression patterns of paired organic anion and cation transporters (OATs and OCTs, respectively). (A) Sequence of the known human OATs and OCTs were aligned with ClustalX and the alignment output was used to generate a dendogram. One thousand bootstrap replicates were performed and the numbers at the branch points indicate that number of times each grouping from the original tree occurred in the replicate trees. Paired genes (Table 2) are enclosed in ellipses. (B) Tissue distributions of paired OATs and OCTs were determined by semi-quantitative PCR on serially diluted cDNAs from 16 human tissues; lane 1 kidney, lane 2 placenta, lane 3 ovary, lane 4 prostate, lane 5 testis, lane 6 liver, lane 7 ileum, lane 8 colon, lane 9 pancreas, lane 10 lung, lane 11 heart, lane 12 muscle, lane 13 spleen, lane 14 thymus, lane 15 WBC, and lane 16 brain. The upper panels for each gene depict amplifications from undiluted template containing ˜1 ng of each cDNA, and the lower panels 10 pg (i.e., from 100-fold diluted template). Amplifications from 100 fg of cDNA. PCRs shown are representative of multiple replicates, and amplifications with human beta actin primers served as controls for sample integrity. Paired OATs and OCTs are schematically depicted above the PCRs. Genes (arrows, with direction indicating orientation) and intergenic regions (solid line segments) are drawn to scale (lower left corner of figure), and their chromosomal locations are indicated. Gene pairs on adjacent chromosomal regions are joined by broken lines.

FIGS. 9A and B show targeted disruption of Oat3 gene. (A) The genomic locus (exons 1-5) and targeting construct for Oat3 are shown. When hybridized with XhaI-digested genomic DNA, the G7 probe detects 6-kb wild-type fragment and a 3 kb mutant fragment. The positions of the PCR primers used to detect the wild-type allele and targeted allele are shown (arrow heads). R. EcoRI; H, HindIII; X, XhaI; rho, XhoI. (B) The Oat3 allelic pattern was analyzed by PCR of genomic DNA. Three different forward primers, a 3 kb one specific for exon 3 of the Oat3 gene (Oat3for) and two specific for the neomycin cassette present in the exon 3 deletion constrict (Neolfor and Neoafor), were each paired with a single reverse primer located in the intron region just prior to exon 4 of Oat3 (K03′). PCR products for the OAT3forKO3′ (a), NeolforKO3′ (b), and NeoBforKO3′ (c) primer pairs are 200, 200, and 230 bp, respectively. Identification of wild-type (wt), heterozygous Oat3^{±} (het), and Oat3^{-/-} knockout (KO) offspring are shown.

FIG. 10A-F shows a histopathological analysis of wild-type and OAT3^{-/-} mouse tissues. Paraffin sections of formalin-fixed tissues from three wild-type and four Oat3 knockout animals were stained with hematoxylin and eosin and examined by light microscopy. Panels A, C, and E are low magnification (X4) images of kidney, liver, and choroid plexus, respectively, from a representative wild-type animal. Panels B, D, and F are low magnification (X4) images of kidney, liver, and choroid plexus, respectively, from a representative Oat3^{-/-} animal. Insets show a region of interest at high magnification (X40) from each of the sections. No morphological abnormalities were observed in any of the animals examined.

FIG. 11A-B shows Northern blot analysis of Oat3 expression in kidney and liver. (A) Approximately 10 pg of total kidney (K) and liver (L) RNA from wild-type (wt), heterozygous (het), and Oat3^{-/-} (KO) littermates was separated by electrophoresis and transferred to a nylon membrane. The membrane was cut into identical halves and exposed to probes generated using either rat OAT1 or mouse Oat3 cDNA as template. No Oat3 mRNA expression was detected in kidney of Oat3^{-/-} mice, but it was readily detected in wild-type and to a lesser degree in heterozygous littermates. No Oat3 signal was detected in liver. Oat1 gene expression was readily detected in the kidney, but not in the liver, of all three animals. The blots were stripped and reprobed with human beta-actin to confirm the integrity of the RNA. The experiment was repeated in two independent sets of wild-type, heterozygous, and Oat3^{-/-} littermates with similar results. (B) To examine sexual dimorphism of Out3 expression in mice, a blot containing total kidney and liver RNA from a male (M) and a female (F) wild-type mouse, a male Oat3^{-/-} mouse, and a male and a female wild-type rat was prepared and screened. Oat3 expression was detected in the kidney of the male and female wild-type mice and rats. Importantly, a faint Oat3 signal was also detected in the male rat liver, but not in the liver of the male mouse. Inclusion of male Oat3 knockout RNA demonstrated specificity of the probes and screening of the blot for 8-actin monitored sample integrity.

FIG. 12 shows PCR analysis of total RNA isolated from CP of adult rat, wild-type and Oat3^{-/-} mice. The RNA was reverse transcribed and used as template for PCR using Oat1-, Oat2-, and Oat3-specific primers that amplify 417-, 325-, and 338 bp products, respectively. Lanes labeled 1, 2, and 3 correspond to Oat1, Oat2, and Oat3 PCR reactions, respectively, from rat, wild-type mice, and Oat 3 knockout mice. A 100-bp ladder is shown. PCR reaction products were obtained for Ot1, Oat2, and Oat3 in wild-type rat and mouse CP, indicating expression of all three organic anion transporters in both species. No native Oat3 gene product was detected in the Oat3^{-/-} mice.

FIG. 13 oocytes three days after injection with either mouse Oat1 or mouse Oat3 cRNA. Oocytes were randomly sorted into test groups and 1 h uptake determined. PAH, mediated uptake of 10 μM [³H]PAH was observed in Oat1- and Oat3-expressing oocytes demonstrating PAH to be a substrate for both Oat1 and Oat3 and establishing the presence of functional transporters in the experimental groups. ES, mediated uptake of 90 nM [³H]estrone sulfate by OAT1 was negligible, whereas Oat3-expressing oocytes exhibited substantial ES transport that was completely blocked by 1 mM probenecid. This confirms ES as a substrate for Oat3, but not Oat1. TC, mediated uptake of 500 nM [³H]taurocholate by Oat3 was readily detected; however, Oat1 failed to support uptake. The experiment was repeated twice with similar results. The data shown are mean values ±S.E. from a single animal (5 oocytes/treatment).

FIG. 14 shows organic anion uptake in renal and hepatic slices. Tissue slices from wild-type and OAT3^{-/-} littermates were incubated for 1 h with substrate ([³H]taurocholate or [³H]para-aminohippurate, 100 nm [³H]lestrone sulfate) in the presence and absence of inhibitors (1 mM bromosulfophthalein or probenecid). Substantial inhibitor-sensitive uptake of taurocholate, estrone sulfate, and PAH was observed in slices from wild-type mouse kidneys. In contrast, renal uptake of each of the substrates was significantly reduced in the knockout animals. Quinine sulfate (Q)-sensitive renal uptake of the organic cation [¹⁴C]TEA was unaffected by Oat3 loss, demonstrating the proper functioning of this related transport system in knockout animals. No significant differences in uptake were measured between hepatic slices from wild-type and Oat-littermates. Experiments were repeated in 3-4 wild-type and knockout littermate pairs, and representative results are shown. Data were calculated as tissue to medium T/M ratios and are presented as mean values ±S. E. (3 slices, treatment). Statistical comparisons (unpaired t test): *, significantly lower than corresponding (wild-type or knockout) control, p<0.05; **, significantly lower than corresponding control, p<0.01.

FIG. 15A-D are confocal images showing FL accumulations in isolated wild-type and Oat3^{-/31} choroid plexus tissue. The CP is composed of capillary projections surrounded by a single layer of cells that protrude into the cerebrospinal fluid-filled ventricles of the brain. The orientation is such that the CSF bathes the apical membrane of the cell and the basal membrane is toward the underlying fenestrated capillary. (A and C) Transmitted light images of wild-type and Oat3^{-/-} CP, respectively, showing the tissue structure. (B and D) Corresponding fluorescence micrographs of the CP shown in (A) and (C). Confocal images were acquired 45 min after exposure to 1 μM FL in the aCSF medium. Panel B, in wild-type CP, note the intracellular concentration of FL above the medium concentration and the fluorescence intensity of the capillaries higher than the cells. Panel C, FL accumulation is markedly lower in the cells and capillaries of Oat3^{-/-} CP. The positions of representative cells and capillaries (CUD) are indicated by arrows. A 20-μm bar is shown.

FIG. 16 shows the quantitation of FL and FL-MTX uptake in intact CP. Fluorescence levels in cells and vessels of CP from 4 wild-type and 4 Out3^{-/-} mice were measured (n=5-10 adjacent cellular and capillary areas/CP). Cellular and capillary FL levels were significantly reduced in CP from Oat3^{-/-} mice as compared with wild-type. No difference in capillary accumulation of FL-MIX was observed between wild-type and Out3^{-/-} CP. Data are given as mean ±S.E. for each animal.

FIG. 17 FL-MTX accumulation in the capillaries of intact wild-type and Oat3^{-/-} choroid plexus. CP were exposed to 2 μM FL-MTX in aCSF for 45 min and subsequently examined by confocal microscopy. The position of representative cells and capillaries (cap) are indicated by arrows. (A and C) Transmitted light images of wild-type and Oat3^{-/-} CP, respectively. (B and D) Corresponding fluorescence micrographs of the CP shown in (A) and (C). Note the lack of concentration of fluorescent signal within the cells and the intense fluorescent signal within the underlying capillaries, in both wild-type and Oat3^{-/-} CP. Photomultiplier gain was turned up slightly to visualize the cells in these images. A 20-μm bar is shown.

FIG. 18 shows the expression of OATs, OCTs and OCTNs in mouse adult olfactory mucosa (Upper panel) and mouse adult kidney (Bottom panel). RT-PCR: OAT, OCT and OAT6 products were amplified by RT-PCR from cDNA derived from olfactory mucosa and kidney. OCT1-2 and OCTN1-3 were detected in olfactory mucosa. OAT1 and OAT6 were detected in olfactory mucosa. Amplification of a G3PDH product was used to control for sample integrity.

FIG. 19 shows the cDNA (SEQ ID NO:35) and predicted amino acid sequence (SEQ ID NO:36) of OAT6. Positions of introns are indicated by the vertical bars transecting the sequence. The 12 putative transmembrane domains (1-12) were assigned on the basis of predicted hydrophobocity

FIG. 20 shows the exon/intron structure of OAT6. Exons are indicated by the rectangles and introns by the lines joining them. The size of exons and introns in bp are indicated below the corresponding exon and intron numbers, respectively. The arrow below the structure indicates the orientation of the gene with respect to its chromosome.

FIG. 21 shows an alignment of the peptide sequence of OAT6 with those of the slc22 family members. Lineage-specific motifs are boxed.

FIG. 22 is a dendrogram of the slc22 family. Sequence of the indicated slc22 family members were aligned with CLUSTAL X and the alignment output was used to generate a dendrogram.

FIG. 23 shows the intron phasing of mouse organic anion and cation transporters. Intron positions and their phases are indicated by ovals, exons and their sizes in base pairs are indicated by boxes. Exons whose sizes are exactly conserved in a majority of OATs, OCTs and OCTNs are indicated by dashes under each group.

FIG. 24 is an E-Blot. Proportional representation of OATs and OCTs in liver, eye, brain and kidney. ESTs are first sorted by tissue. Kidney: 93396 ESTs, Brain: 39024 ESTs, Liver: 80333 ESTs, Eye: 96322 ESTs.

FIG. 25 shows expression of OAT6 in adult and fetal mouse. OAT6 specific products were amplified by RT-PCR from cDNAs derived from the indicated adult and fetal mouse tissues (7-17 day embryo). Panel A (adult): OAT6 was detected in olfactory mocusa (OM) and testis. Amplifications of a G3PDH product were used to control for sample integrity. PCRs shown are representative of multiple replicates. Panel B (fetal): expression of OAT6 was detected at 7 day embryo.

### DETAILED DESCRIPTION

Organic anion and cation transporters, slc22 family members (OATs, OCTs, OCTNs, and ORCTLs) are transmembrane proteins essential to renal excretion and are encoded by a group of related genes. Multiple OATs and OCTs have been identified in the last few years. Examples of these organic anion transporters include:

| Organisms | OAT (aliases) | GenBank Accession |
|---|---|---|
| Murine | OAT 1 (NKT slc22a6) | MMU52842 and NM008766 |
| Murine | OAT 2 (NLT slc22a7) | AB069965 |
| Murine | OAT 3 (Roct slc22a8) | NM_031194 and AB079895 |
| Human | OAT 1 | AF097490 |
| Human | OAT 2 | AF210455 |
| Human | OAT 3 | AF097491 |
| Human | OAT 4 | AB026116 |
| Human | OAT 5 | BK001421 |
| Murine | OAT 6 | SEQ ID NO: 35 and 36 |

The above identified GenBank references are incorporated herein by reference in the entirety.

Additionally, UST1, UST3, and OAT5, have sequence homology to transport organic anions as well. Examples of organic cation transporters include OCT1 (slc22a1), OCT2 (slc22a2), and OCT3 (slc22a3), while OCTN1 (slc22a4), OCTN2 (slc22a5; UST2), OCTN3 (slc22a9), and CT2 transports carnitine as well as cations.

Active transport of endogenous metabolites and xenobiotics from blood to urine across the cells of the renal proximal tubule is an important protective mechanism. Accordingly, there are excretory transport systems in the kidney comprising groups of organic anion transporters (OATs) and organic cation transporters (OCTs), which are subfamilies within the amphiphilic solute transporter branch (SLC22A) of the major facilitator superfamily. In the adult, these transporters are also expressed in other barrier epithelia such as the intestine, placenta, retinal pigment epithelium, and the choroid plexus (CP). Their expression in the CP (located in the ventricles of the brain), coupled with evidence that neurotransmitters (e.g., choline) and neurotransmitter metabolites (e.g., 5-hydroxyindoleacetic acid (from serotonin) and homovanillic acid (from dopamine)) are substrates for the OATs and OCTs, suggests that these transporters actively regulate the composition of brain extracellular fluid. This regulation of the extracellular fluid is accomplished by controlling the flux of xenobiotics and central nervous system by-products from cerebrospinal fluid (CSF) to blood. Moreover, during development the spatiotemporal pattern of renal OAT expression suggests that these genes may be useful in understanding the mechanisms of proximal tubule maturation. Transient OAT expression in unexpected sites (e.g., spinal cord, bone, and skin) during development may indicate that these transporters play a critical role in the formation or preservation of extrarenal tissues, as well. Thus, elucidation of the specific mechanisms regulating OAT expression may provide insight into the processes controlling development, CSF-blood equilibrium, and drug handling capacity in the kidney.

Six members of the organic anion transporter family have been characterized thus far: Oat1, Oat2, Oat3, Oat4, OATS and OAT6. Oat1, originally described as novel kidney transporter, NKT, (GenBank accession no. MMU52842 (murine), incorporated herein by reference), has been localized to the basolateral membrane of renal proximal tubules and to the apical membrane of CP through direct observation of an Oat1/green fluorescent protein fusion construct and by immunohistochemistry on adult rat kidney sections. Uptake by Oat1 is trans-stimulated by glutarate, demonstrating that it functions as an organic anion/dicarboxylate exchanger, consistent with its localization in the basolateral membrane of proximal tubule cells. Initial characterization studies of Oat2 (originally described as novel liver transporter), Oat3, and Oat4 indicated that, unlike Oat1, uptake mediated by these transporters is not subject to trans-stimulation, indicating that they function as facilitative transporters rather than exchangers. Mechanistically this would suggest that these transporters are located in the apical membrane in the proximal tubule; however, human OAT3 has recently been localized to the basolateral membrane by immunocytochemistry.

Oat3 (Slc22a8) was originally identified as a gene of unknown specificity that had sequence homology to the transporter genes Oat1 and Oat2. It was subsequently demonstrated that its expression is absent in the juvenile cystic kidney (jck) mouse model and markedly reduced in the kidneys of mice homozygous for the osteosclerosis (oc) mutation. It was, therefore, designated as “reduced in osteosclerosis transporter,” or Roct. However, it is now known that Roct shares a 92 and 64% identity at the amino acid level with the recently cloned rat and human Oat3 genes, respectively, and is the murine Oat3 ortholog.

Organic transporters play critical roles in drug clearance and metabolism in vivo. Thus, by determining the role of specific organic transporters one can modify drug clearance and metabolism by, for example, inhibiting one or more specific organic transporters or by modifying a specific drug such that it is not cleared by a specific organic transporter.

Organic anion transport is known, on the basis of physiological studies, to be regulated by steroid particularly androgens, as well as by the substrates themselves. Investigations to date have revealed a marked sexual dimorphism in OAT expression, with OAT2 and OAT3 messenger RNA levels negatively and positively regulated, respectively, by testosterone. The potential implications of these findings for gender differences in drug-handling in humans are clear. Post-translationally, OATS have been found to be regulated by phosphorylation: epidermal growth factor, acting through mitogen-activated protein kinases induces OAT activity, and protein kinase C.

Endogenous OAT substrates, which include cyclic nucleotides, prostaglandins, folate, and of course dicarboxylates, suggest the potential functioning of OATs in various cellular and physiological processes. In addition, certain unexpected observations on the ontogeny of the OATs hint at a potential role in development (possibly due to morphogenetic activity of the above substrates). For example, OATS 1-3 manifest transient embryonic expression in a variety of disparate tissues, including brain, spinal cord, dura matter, intestine, lung, skin, and bone, in addition to liver and kidney. Study of the evolution of OATs through identification of orthologs in phylogenetically distant (and simpler) organisms might provide clues to any additional functions performed by these genes. A search of the recently completed Cuenorhabditis elegans (worm) and Drosophila melanogaster (fly) genomes and have found several OAT-like sequences. Because each gene in the entire C. elegans genome has been systematically inactivated with RNA interference, null mutations for the putative worm OATs are available for developmental and functional analysis.

In addition, the sequencing of the human genome has uncovered a remarkable feature of the chromosomal organization of OAT genes. Six of the eight known OATs are found in three tightly linked pairs (i.e., as adjoining neighbors without other genes interposed between them); specifically, these are OAU and OAT1 and 3, OAT4 and URAT1, and UST3 and OAT5. Inspection of the dendrogram of the OAT family reveals that these physical pairs are also proximal tubule closely related ‘phylogenetic pairs’ (FIG. 2). Furthermore, pair members have similar tissue distributions: OAT1 and 3 are in kidney and to a lesser extent brain; OAT4 and URAT1 are also in kidney, but not in brain, with OAT4 present in placenta as well: OAT5 and UST3 are in liver. These observations suggest that the pairing of OAT genes might exist to facilitate the coordinated transcription (co-regulation) of pair members, for example, through their utilization of a shared regulatory DNA sequence.

Human OAT1 and 3 and rat OAT1 and 3 were all specifically detected in the basolateral concentrations of the proximal tubule in agreement with previous observations, and where investigated. Expression of OAT 1 and 3 was found throughout S1-S3 (in contrast to earlier studies suggesting that rat OAT1 was restricted to the S2 segment). However, rat OAT3 was additionally found in the cortical and medullary thick ascending loop of Henle, connecting tubules, and cortical and medullary collecting ducts. Human OAT4 and URAT1 resembled human OAT1 and 3 in being exclusive to the proximal tubule, but were localized to the apical rather than basolateral surface.

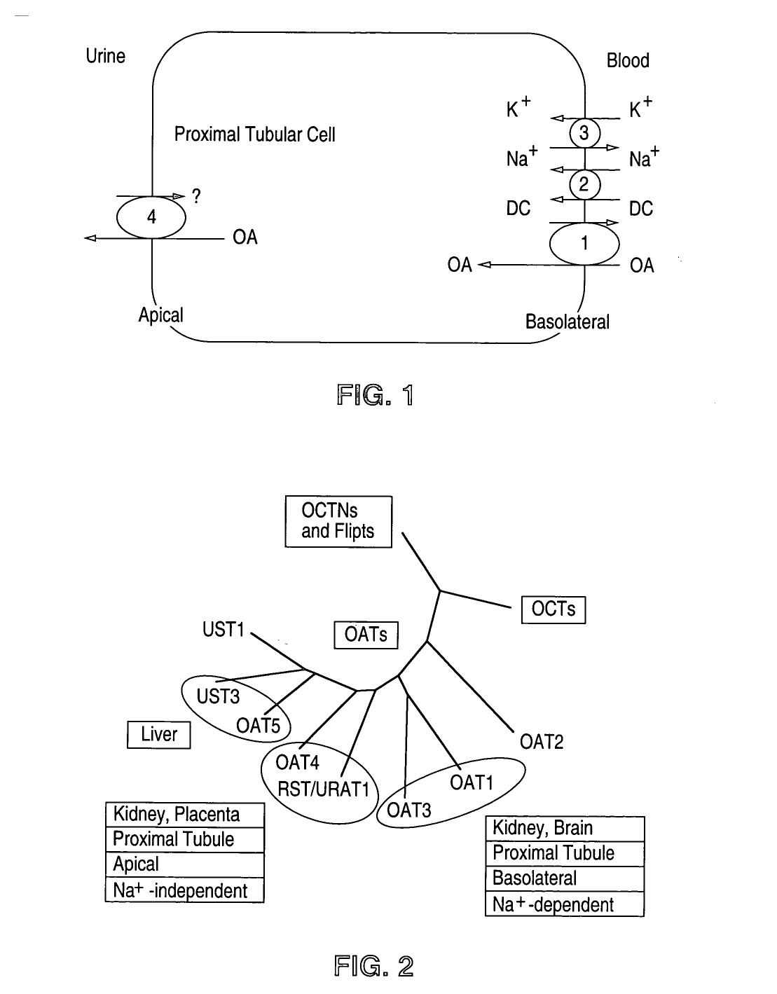

Therefore, tubular and membrane localization does appear to sort with the chromosomal pairings, with OAT1 and 3 at the basolateral surface of the proximal tubule and OAT4 and URAT1 at its apical surface (FIG. 2). Consistent with what is known about the physiology of basolateral and apical organic anion transport (FIG. 1), the basolateral pair, OAT1 and OAT3, couple organic anion influx to the sodium-dependent dicarboxylate gradient, while the apical pair, URAT1 (which couples organic anion efflux to uptake of tubular urate) and OAT4, are sodium-independent. Thus the OAT1/OAT3 and OAT4/URAT1 gene pairs appear to operate at the basolateral and apical steps respectively of tubular renal organic anion secretion. It should be noted that the membrane colocalization of pair-members does not imply their functional interdependence, as individual OATS are known to independently mediate organic anion transport.

The availability of molecular clones for the OATs has enabled the rapid (and continuing) functional characterization of these transporters. Notable among recently identified substrates is urate, the tubular reabsorption of which appears to be due to exchange by the apically located URAT1 for intracellular organic anions. Urate, and therefore URATI, have been proposed to contribute to the relative longevity of humans. However, though not noted as such in the report of its cloning, URAT1 appears to be the human ortholog of the previously identified murine RST. The presence of an ortholog in the comparatively short-lived mouse mitigates the hypothesis that URAT1 makes a major contribution to human longevity. Other recently reported substrates include uremic toxins (including indoxyl sulfate which has been hypothesized to contribute to the progression of renal failure), mercaptopurates, and the heavy metal chelator 2,3-dimercapto-1-propanesulfonate.

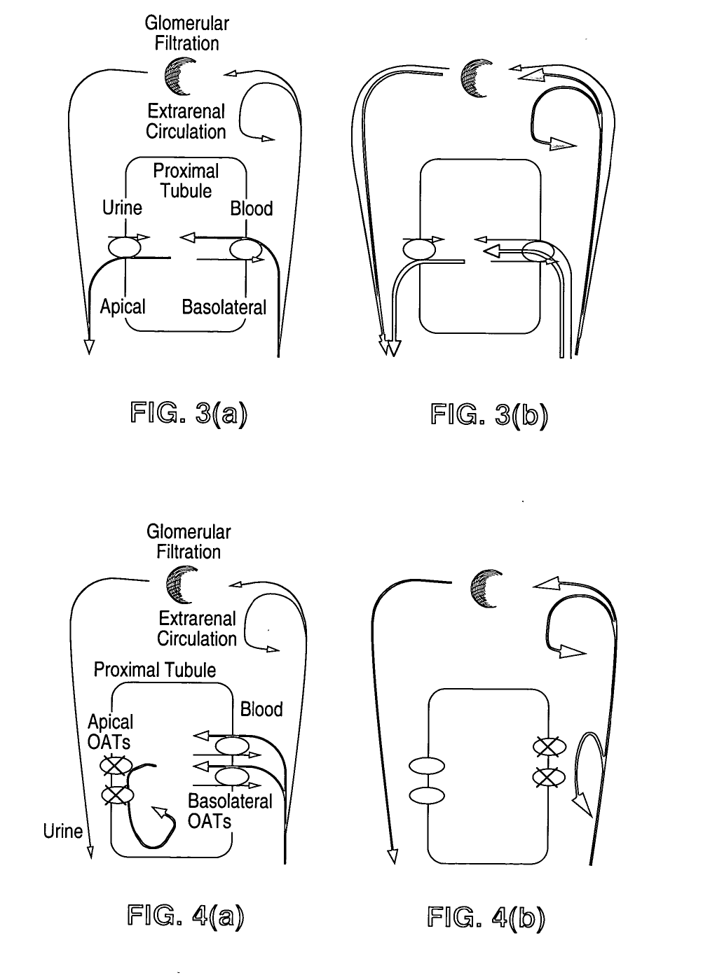

Nephrotoxins as substrates for OATS have turned out to be a recurring theme, as exemplified by the transport of ochratoxin A, cephaloridine, tetracycline, mercuric conjugates, nephrotoxic cysteine conjugates, and the antivirals adefovir and cidofovir (the latter are the topic of much current interest because of their potential role in the treatment of smallpox following a bioterror attack). Thus, the proximal tubule might be a primary target for toxicity precisely because potential toxins accumulate within it through the action of the basolateral OATS. Toxicity might therefore be expected to be reduced with OAT inhibitors or competitive substrates (FIG. 3). Indeed, this appears to be the mechanism underlying the protective action of probenecid (the traditionally used OAT competitive inhibitor)and NSAIDs (which are known OAT substrates) against toxicity from adefovir and cidofovir, cephaloridine, ochratoxin A, and mercury.

Although cloning of organic transporters is a first step to understanding the function of the protein, such in vitro and in silico studies do not provide a full understanding of a protein's function. In vivo functional analysis can be achieved by gene knockout techniques in mammalian systems (e.g., in mice, rats, and the like). The direct approach to elucidation of the in vivo function of the OATs is of course through generation of the corresponding knock-out mice. Thus, the invention provides knockout non-human organisms lacking one or more (typically at least two, “double knockouts”) OAT genes.

“Knock-in” refers to the fusion of a portion of a wild-type gene to the cDNA of a heterologous gene

“Knock-out” refers to partial or complete suppression of the expression of a protein encoded by an endogenous DNA sequence in a cell. The “knock-out” can be affected by targeted deletion of the whole or part of a gene encoding a protein, in an embryonic stem cell. As a result, the deletion may prevent or reduce the expression of the protein in any cell in the whole animal in which it is normally expressed. For example, an “OAT3 knock-out animal” refers to an animal in which the expression OAT3 has been reduced or suppressed by the introduction of a recombinant nucleic acid molecule that disrupts at least a portion of the genomic DNA sequence encoding OAT3.

“Transgenic animal” refers to an animal to which exogenous DNA has been introduced while the animal is still in its embryonic stage. In most cases, the transgenic approach aims at specific modifications of the genome, e.g., by introducing whole transcriptional units into the genome, or by up- or down-regulating pre-existing cellular genes. The targeted character of certain of these procedures sets transgenic technologies apart from experimental methods in which random mutations are conferred to the germline, such as administration of chemical mutagens or treatment with ionizing solution.

The term “knockout mammal” and the like, refers to a transgenic mammal wherein a given gene has been suppressed by recombination with a targeting vector. It is to be emphasized that the term is intended to include all progeny generations. Thus, the founder animal and all F1, F2, F3, and so on, progeny thereof are included.

The term “chimera,” “mosaic,” “chimeric mammal” and the like, refers to a transgenic mammal with a knockout in some of its genome-containing cells.

The term “heterozygote,” “heterozygotic mammal” and the like, refers to a transgenic mammal with a knockout on one of a chromosome pair in all of its genome-containing cells.

The term “homozygote,” “homozygotic mammal” and the like, refers to a transgenic mammal with a knockout on both members of a chromosome pair in all of its genome-containing cells.

A “non-human animal” of the invention includes mammals such as rodents, non-human primates, sheep, dog, cow, chickens, amphibians, reptiles, etc. Typical non-human animals are selected from the rodent family including rat and mouse, most typically mouse, though transgenic amphibians, such as members of the Xenopus genus, and transgenic chickens can also provide important tools for understanding and identifying agents which can affect, for example, protein function and disease models.

In the animals of the invention, at least two OAT genes, at least two OCT genes or an OAT/OCT gene are mutated such that the animals do not express the functional gene products. In one aspect, the double knockout comprises a basolateral OAT gene and an apical OAT gene. In another aspect, the knockout comprises two basolateral OAT genes or two apical OAT genes.

A “mutation” is a detectable change in the genetic material in the animal, which is transmitted to the animal's progeny. A mutation is usually a change in one or more deoxyribonucleotides, the modification being obtained by, for example, adding, deleting, inverting, or substituting nucleotides.

Typically, the genome of the transgenic non-human mammal comprises one or more deletions in one or more exons of the genes and further comprises a heterologous selectable marker gene.

In principle, knockout animals may have one or both copies of the gene sequence of interest disrupted. In the latter case, in which a homozygous disruption is present, the mutation is termed a “null” mutation. In the case where only one copy of the nucleic acid sequence of interest is disrupted, the knockout animal is termed a “heterozygous knockout animal”. The double knockout animals of the invention are typically homozygous for the disruption of both OAT genes being targeted.

It is important to note that it is not necessary to disrupt a gene to generate a transgenic organism lacking functional expression. The invention includes the use of antisense molecules that are transformed into a cell, such that production of an OAT polypeptide is inhibited. Such an antisense molecule is incorporated into a germ cell as described more fully herein operably linked to a promoter such that the antisense construct is expressed in all cells of a transgenic organism.

Techniques for obtaining the transgenic animals of the invention are well known in the art; the techniques for introducing foreign DNA sequences into the mammalian germ line were originally developed in mice. One route of introducing foreign DNA into a germ line entails the direct microinjection of linear DNA molecules into a pronucleus of a fertilized one-cell egg. Microinjected eggs are subsequently transferred into the oviducts of pseudopregnant foster mothers and allowed to develop. About 25% of the progeny mice inherit one or more copies of the micro-injected DNA. Currently, the most frequently used techniques for generating chimeric and transgenic animals are based on genetically altered embryonic stem cells or embryonic germ cells. Techniques suitable for obtaining transgenic animals have been amply described. A suitable technique for obtaining completely ES cell derived transgenic non-human animals is described in WO 98/06834.

To generate the animals of the invention, in the first step, transgenic animals are generated that lack an OAT1-5 or 6 function or an OCT function. Such animals can be obtained by standard gene targeting methods as described above, typically by using ES cells. In one aspect, the transgenics can be intercrossed to obtain a double knockout mice.

In another aspect, serial embryonic stem cell knockouts are used to obtain the double knockout. Thus, in a further aspect, the invention relates to a method for producing a double knockout non-human mammal comprising (i) providing an embryonic stem (ES) cell from the relevant animal species comprising a first intact OAT gene; (ii) providing a first targeting vector capable of disrupting the first intact OAT gene; (iii) introducing the first targeting vector into the ES cells under conditions where the intact first OAT undergoes homologous recombination with the first targeting vector to produce a mutant first OAT gene; (iv) introducing the ES cells carrying a disrupted first OAT gene into a blastocyst; (v) implanting the blastocyst into the uterus of pseudopregnant female; (vi) delivering animals from said females, identifying a first mutant animal that carries the mutant allele and obtaining mutant ES cells from the first mutant animal; (v) providing a second targeting vector capable of disrupting a second intact OAT gene; (vi) introducing the second targeting vector into the mutant ES cells under conditions where the intact second OAT gene undergoes homologous recombination with the second targeting vector to produce a mutant second OAT gene; (vii) introducing the mutant ES cells carrying a disrupted second OAT gene into a blastocyst; (viii) implanting the blastocyst into the uterus of pseudopregnant female; (ix) delivering animals from said females,; and (x) selecting for OAT double knockout animals and breeding them.

A “targeting vector” is a vector comprising sequences that can be inserted into the gene to be disrupted, e.g., by homologous recombination.

The targeting vector generally has a 5′ flanking region and a 3′ flanking region homologous to segments of the gene of interest, surrounding a foreign DNA sequence to be inserted into the gene. For example, the foreign DNA sequence may encode a selectable marker, such as an antibiotics resistance gene. Examples for suitable selectable markers are the neomycin resistance gene (NEO) and the hygromycin β-phosphotransferase gene. The 5′ flanking region and the 3′ flanking region are homologous to regions within the gene surrounding the portion of the gene to be replaced with the unrelated DNA sequence. DNA comprising the targeting vector and the native gene of interest are contacted under conditions that favor homologous recombination. For example, the targeting vector and native gene sequence of interest can be used to transform embryonic stem (ES) cells, in which they can subsequently undergo homologous recombination.

Thus, a targeting vector refers to a nucleic acid that can be used to decrease or suppress expression of a protein encoded by endogenous DNA sequences in a cell. In a simple example, the knockout construct is comprised of an OAT polynucleotide, such as the OAT1 polynucleotide sequence, with a deletion in a critical portion of the polynucleotide so that a functional OAT1 cannot be expressed therefrom. Alternatively, a number of termination codons can be added to the native polynucleotide to cause early termination of the protein or an intron junction can be inactivated. In a typical knockout construct, some portion of the polynucleotide is replaced with a selectable marker (such as the neo gene) so that the polynucleotide can be represented as follows: OAT1 5′/neo/OAT1 3′, where OAT1 5′ and OAT1 3′, refer to genomic or cDNA sequences which are, respectively, upstream and downstream relative to a portion of the OAT1 polynucleotide and where neo refers to a neomycin resistance gene.

Proper homologous recombination can be confirmed by Southern blot analysis of restriction endonuclease digested DNA using, as a probe, a non-disrupted region of the gene. Since the native gene will exhibit a restriction pattern different from that of the disrupted gene, the presence of a disrupted gene can be determined from the size of the restriction fragments that hybridize to the probe.

In an animal obtained by the methods above, the extent of the contribution of the ES cells that contain the disrupted first OAT gene or the second OAT gene to the somatic tissues of the transgenic animal can be determined visually by choosing animal strains for the source of the ES cells and blastocyst that have different coat colors.

In a one embodiment, the double knockout animals of the invention are mice. In other embodiments of this invention, the animals are rats, guinea pigs, rabbits, non-human primates or dogs. The production of knockout is described in further detail below.

The invention further provides for transgenic animals, which can be used for a variety of purposes, e.g., to identify therapeutics agents for OAT mediated disorders associated with, for example, drug toxicity, uptake and clearance.

The transgenic animals can typically contain a transgene, such as reporter gene, under the control of an OAT promoter or fragment thereof. Methods for obtaining transgenic and knockout non-human animals are well known in the art. Knock out mice are generated by homologous integration of a “targeting vector” construct into a mouse embryonic stem cell chromosome which encodes a gene to be knocked out. In one embodiment, gene targeting, which is a method of using homologous recombination to modify an animal's genome, can be used to introduce changes into cultured embryonic stem cells. By targeting an OAT gene of interest in ES cells, these changes can be introduced into the germlines of animals to generate chimeras. The gene targeting procedure is accomplished by introducing into tissue culture cells a DNA targeting vector that includes a segment homologous to a target OAT locus, and which also includes an intended sequence modification to the OAT genomic sequence (e.g., insertion, deletion, point mutation). The treated cells are then screened for accurate targeting to identify and isolate those which have been properly targeted.

Generally, the embryonic stem cells (ES cells) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. Mol. Biol. 87:27-45). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934). Still another ES cell line is the WW6 cell line (Ioffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. (1986)).

A targeting vector construct refers to a uniquely configured fragment of nucleic acid which is introduced into a stem cell line and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art. A typical targeting vector contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid which encodes a positive selectable marker, such as the neomycin resistance gene (neoR). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for OAT or other gene of interest to be knocked out. When the resulting construct recombines homologously with the chromosome at this locus, it results in the loss of the omitted coding sequence, otherwise known as the structural gene, from the genomic locus. A stem cell in which such a homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgenic animal which expresses a polypeptide of the targeted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. One skilled in the art will be familiar with useful markers and the means for detecting their presence in a given cell.

As mentioned above, the homologous recombination of the above described “knock out” and “knock in” constructs is sometimes rare and such a construct can insert nonhomologously into a random region of the genome where it has no effect on the gene which has been targeted for deletion, and where it can potentially recombine so as to disrupt another gene which was otherwise not intended to be altered. Such non-homologous recombination events can be selected against by modifying the above-mentioned targeting vectors so that they are flanked by negative selectable markers at either end (particularly through the use of two allelic variants of the thymidine kinase gene, the polypeptide product of which can be selected against in expressing cell lines in an appropriate tissue culture medium well known in the art—i.e. one containing a drug such as 5-bromodeoxyuridine). Non-homologous recombination between the resulting targeting vector comprising the negative selectable marker and the genome will usually result in the stable integration of one or both of these negative selectable marker genes and hence cells which have undergone non-homologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques which are well known to those familiar in the art. Alternatively, PCR can be used.

Each targeting vector to be inserted into the cell is linearized. Linearization is accomplished by digesting the DNA with a suitable restriction endonuclease selected to cut only within the vector sequence and not the 5′ or 3′ homologous regions or the selectable marker region.

For insertion, the targeting vector is added to the ES cells under appropriate conditions for the insertion method chosen, as is known to the skilled artisan. For example, if the ES cells are to be electroporated, the ES cells and targeting vector are exposed to an electric pulse using an electroporation machine and following the manufacturer's guidelines for use. After electroporation, the ES cells are typically allowed to recover under suitable incubation conditions. The cells are then screened for the presence of the targeting vector as explained herein. Where more than one construct is to be introduced into the ES cell, each targeting vector can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however the typical method is by microinjection. For microinjection, about 10-30 cells are collected into a micropipet and injected into embryos that are at the proper stage of development to permit integration of the foreign ES cell containing the recombination construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan.

While any embryo of the right stage of development is suitable for use, typical embryos are male. In mice, the typical embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the OAT gene knocked out in various tissues of the offspring by probing the Western blot with an antibody against the particular OAT protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of an OAT gene can be controlled by recombinase sequences.

Animals containing more than one knockout construct and/or more than one transgene expression construct are prepared in any of several ways. A typical manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

In another aspect, a transgenic animal can be obtained by introducing into a single stage embryo a targeting vector. The zygote is the best target for micro-injection. In the mouse, the male pronucleus reaches the size of approximately 20 micrometers in diameter which allows reproducible injection of 1-2pl of DNA solution. The use of zygotes as a target for gene transfer has an advantage in that in most cases the injected DNA will be incorporated into the host gene before the first cleavage (Brinster et al. (1985) PNAS 82:4438-4442). As a consequence, all cells of the transgenic animal will carry the incorporated nucleic acids of the targeting vector. This will in general also be reflected in the efficient transmission to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus. In some species such as mice, the male pronucleus is typically used. Typically the exogenous genetic material be added to the male DNA complement of the zygote prior to its being processed by the ovum nucleus or the zygote female pronucleus. It is thought that the ovum nucleus or female pronucleus release molecules which may affect the male DNA complement, perhaps by replacing the protamines of the male DNA with histones, thereby facilitating the combination of the female and male DNA complements to form the diploid zygote.

Thus, the exogenous genetic material is typically added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the a exogenous-nucleic acid (e.g., a targeting vector) into the embryo may be accomplished by any means known in the art such as, for example, microinjection, electroporation, or lipofection. Following introduction of the exogenous nucleic acid into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is used. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of a transgene (e.g., the exogenous genetic material or targeting vector constructs) which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of a targeting vector construct, in order to insure that one copy is functional.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of an exogenous polynucleotide (e.g., that of a targeting vector) by any suitable method as described herein. Alternative or additional methods include biochemical assays such as enzyme and/or immunological assays, histological stains for particular marker or enzyme activities, flow cytometric analysis, and the like.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different knockout, or both. Alternatively, the partner may be a parental line. Where in vitro fertilization is used, the fertilized embryo may be implanted into a surrogate host or incubated in vitro, or both. Using either method, the progeny may be evaluated using methods described above, or other appropriate methods.

Retroviral infection can also be used to introduce a targeting vector into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the targeting vector is typically a replication-defective retrovirus carrying the exogenous nucleic acid (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the targeting vector (e.g., the exogenous nucleic acids) since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

In another aspect, the invention relates to the use of an OAT, OAT/OCT, OCT double knockout animal, in particular a mouse, as a model to study drug metabolism and clearance.

In a further embodiment, the invention relates to cells and tissues that carry mutations in at least two OAT, OCT, or OAT/OCT genes. The cells can be primary cells or established cell lines obtained from the transgenic animals of the invention according to routine methods, i.e. by isolating and disintegrating tissue, in particular kidney tissue, blood brain barrier tissue, and the like, from the double knockout animal and passaging the cells.

Such cells and tissues derived from the animals of the invention, in which the activity of at least two OATs, at least two OCTs, or an OAT and OCT has been reduced or abolished, are useful in in vitro methods relating to drug clearance and metabolic studies and the functional analysis of OATs and OCTs.

In a further aspect, the invention relates to a method for determining whether a compound has cytotoxic potential, wherein a candidate compound is administered, for example, to an OAT double knockout animals and the ability of the compound to be metabolized or cleared from the animal's system is analyzed, wherein highly toxic effects will be readily apparent by determining the animal's survival.

The test compound can be administered to the non-human double knockout animal in a variety of ways, e.g. orally, in a suitable formulation, by parenteral injection, subcutaneous, intramuscular, or intra-abdominal injection, infusion, ingestion, suppository administration, and skin-patch application. The effect of the compound on, for example, kidney metabolism or brain tissue accumulation can be determined using methods well known to a person of ordinary skill in the art by analyzing the test compound in various tissues of the animal as well as conditions associated with cytotoxicity including apoptosis of cells in the tissues.

In an alternative method for screening compounds the test compounds can be contacted with cells derived from such double knockout animals. In such methods, cells are incubated with the compound. A compound that does not have cytotoxic effects on the cells is a compound that may be further assessed with regard to its therapeutic and/or cytotoxic effect by administering the compound to a double knockout animal as described above.

Cell lines derived from the double knockout animals are further useful to dissect the physiological and biochemical functions of various OAT pairs, OCT pairs and combinations thereof.

In one aspect the transgenic animals of the invention provides an animal model for studying drug clearance and toxicity. The model comprises a transgenic mouse whose genome contains a homozygous disruption of at least two OATs or OCTs or an OAT and an OCT. In one specific aspect, the double knockout comprises a homozygous knockout of two basolateral OATS (e.g., OATS 1 and 3). Knocking out of the basolateral pair (OAT1 and OAT3) can result in decreased uptake of substrates and thus decreased nephrotoxicity. However, lack of basolateral OAT activity can also result in delayed clearance of substrates, increasing extrarenal toxicity (FIG. 4).

In another aspect the double knockout comprises a homozygous knockout of two apical OATs (e.g., URAT1 and OAT4). Conversely, knocking out the apical pair (OAT4 and RST) can result in substrates accumulating in the proximal tubule, due to the unopposed action of the basolateral OATS, and thus in increased nephrotoxicity.

In another aspect, the double knockout comprises a homozygous knockout for one apical and one basolateral OAT (e.g., OAT1 and RST, OAT1 and OAT4, OAT3 and RST, or OAT3 and OAT4). The transgenic double knockout mouse of the invention displays at least one sign or symptom associated with dysfunctional anionic transport including, for example, the inability to secrete organic anions. Such measurements of organic anion transport can be performed using routine skill in the art.

The invention demonstrates that knockout of OAT3 results in specific decreases in kidney and choroid plexus secretion of organic anions, confirming the importance of this transporter for organic anion transport in vivo. No obvious morphological defects were noted in OAT3 knockout mice, this cannot be taken as evidence for the lack of a developmental role for OAT3, given the likelihood that other members of the OAT family, especially OAT1 (which, as noted above, is paired and largely co-expressed with OAT3), might confer functional redundancy. Knockouts of multiple OAT genes, particularly double knockouts of OAT gene pairs, can overcome such redundancy and yield fundamental insights into OAT function.

These data indicate a key role for Oat3 in systemic detoxification and in control of the organic anion distribution in cerebrospinal fluid. Thus, the resultant Oat3^{-/-} mice are fertile and exhibit no obvious morphological defects, but present a distinct physiological phenotype measurable as impaired organic anion transport function in renal and choroid plexus epithelia. This reduced transport capacity indicates that Oat3 plays an essential role in the disposition of organic anions in the general circulation and in the extracellular environment of the brain.

The invention also provides methods of identifying polymorphisms associated with rug toxicity associated with OATs, OCTs and combinations thereof. Variability in drug handling is a leading cause of morbidity and mortality, with approximately 2.2 million severe adverse drug reactions (ADRs) and 106 000 deaths attributable to ADRs in the lJS each year. Such statistics have provided impetus for the field of pharmacogenetics, which seeks broadly to correlate genetic variations the most frequent of which are single nucleotide polymorphisms (SNPs) with pharmacokinetic and pharmacodynamic parameters in order to help predict the risk of adverse events. While the cytochrome P450 enzymes have been the focus of much prior research in this area, the role of excretory molecules such as transporters is increasingly appreciated. In fact, given the variety and clinical importance of the pharmaceuticals that interact with OATs, they might be considered as the “cytochromes of the kidney”, meriting investigation of the potential relationship between OAT SNPs and the clinical response to a broad variety of pharmaceuticals. Cidofovir, for example has been proposed for the treatment of smallpox infections. However, there is concern about the potential use of this drug in treating a large number of otherwise healthy people (for example in the event of a bioterrorist attack) because of its nephrotoxicity. As noted above, this toxicity appears to be related to the action of OATs, suggesting that SNPs in these genes may predict a predisposition to such an adverse reaction. Given the prospect of treating many thousands of people with cidofovir, testing for variants in OATs has the potential to drastically diminish the incidence of nephrotoxic events. Similarly, two of the most commonly used classes of antihypertensive medications (ACE inhibitors and diuretics) are handled by OATs; the therapeutic activity of the latter might be directly related to transport by OATs into the tubular lumen. SNPs affecting the function of these transporters may account for much of the variation in the response to these antihypertensives. Given that only one-third of all hypertensive patients are adequately treated, the ability to predict response a priori will likely translate to a dramatic reduction in the debilitating consequences of poorly controlled blood pressure, cardiovascular, cerebrovascular and renal disease.

A number of OAT SNPs have been compiled. Discovery of critical SNPs will be assisted by knowledge of the position of the specific sub-sequences within OAT genes that are important for their structure/function or (transcriptional or post-transcriptional) regulation.

In this regard, traditional molecular biological and biochemical approaches to determining the relationship between gene sequences and their biological activity may serve as a vital preliminary to pharmacogenetic analyses. Along these lines, various investigators have recently begun the process of generating targeted mutations in order to identify the amino-acid residues important for Oxl'function. Comparative genomics provides a complementary approach to the identification of such residues (as they are likely to be highly conserved). Eventually, a list of critical OAT SNPs could be compared with an individual patient's genetic profile to potentially guide drug therapy.

In yet another aspect, the invention provides methods of modulating OAT expression. A computational analysis of the murine and human OAT1-genomic locus detected numerous conserved binding sites for several factors of known importance in the differentiation of the kidney, including Paxl, Pbx, Tcf, Wilms' tumor suppressor (WT1), and hepatocyte nuclear factor 1 (HNF1), any among which might play a role in the potential transcriptional co-regulation of the OAT1 and 3 gene pair. HNF1 is a particularly plausible candidate regulator of transporter gene expression, as it induces the transcription of other renal transporters, including the sodium Substrates, phosphate cotransporter (NaPi) and the type II sodium-glucose cotransporter (SGLT2). In addition, HNF1 knockout mice are a model of Fanconi syndrome (proximal tubule dysfunction resulting in urinary wasting of glucose, amino acids, and phosphate). One might accordingly predict that these knockouts manifest effective renal secretion of organic anions as well.

As yet there have been no studies of the transcriptional regulation of this important gene family. While such studies have traditionally been labor-intensive comparative genomics approaches are now available that have proven reliable guides to critical regulatory elements. The genomic sequencing of murine OAT1 (previously referred to as NKT) and OAT3 (Roct) and derivation of phylogenetic footprints (evolutionarily conserved non-coding sequences) by comparison to the human genome identified binding sites within these footprints for several transcription factors implicated in kidney development including PAX1 PBX WT1 and HNF1. Additionally, OATs and OCTs occur in the human and mouse genomes as tightly linked pairs (OAT1 and OAT3, UST3 and OAT5, OAT4 and URAT1/RST, OCT1 and 2, OCTN1 and 2, ORCTL3 and 4) that are also close phylogenetic relations with Flipt1 and 2 and OAT2 the only unpaired family members. The pair-members have similar tissue distributions suggesting that the pairing might exist to facilitate the co-regulation of the genes within each pair.

As of yet there have not been any investigations of the transcriptional regulation of OATs and OCTs. Such studies have traditionally involved exhaustive evaluation of large stretches of non-coding sequences in search of the relatively small regulatory elements hidden within them. However in the post-genomic era computational analyses such as phylogenetic footprinting (identification of evolutionarily conserved regions within non-coding sequences) and transcription factor binding site searches can e used to predict likely regulatory elements; these can then be prioritized for experimental verification greatly expediting analysis of transcriptional regulation.

The invention also provides putative transcriptional binding sites. The methods of the invention have used the genomic sequence of murine OAT1 and 3 and derived phylogenetic footprints by comparison to the publicly available human orthologs. Putative transcription factor binding sites were identified within these footprints representing potential regulatory elements. Promisingly many among these sites are recognized by factors important in differentiation of the kidney (the location of greatest OAT1 and 3 expression) including PAX1 PBX WT1 and HNF1. Furthermore in determining the chromosomal locations of OATs and OCTs it is noted a remarkable feature of their genomic organization: 12 of 16 human family members are co-localized with their nearest paralogs in six tightly linked pairs. Pair-members were found that have approximately similar expression patterns. These findings suggest that the pairing might exist to facilitate the co-regulation of pair members.

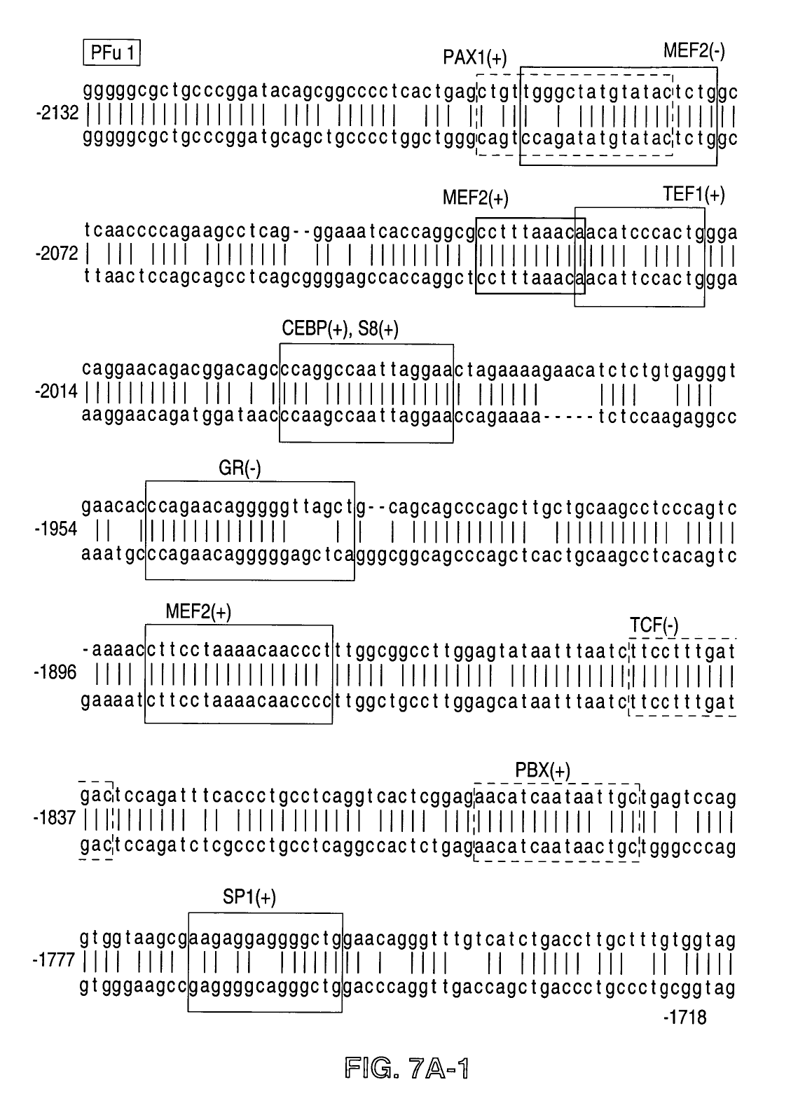

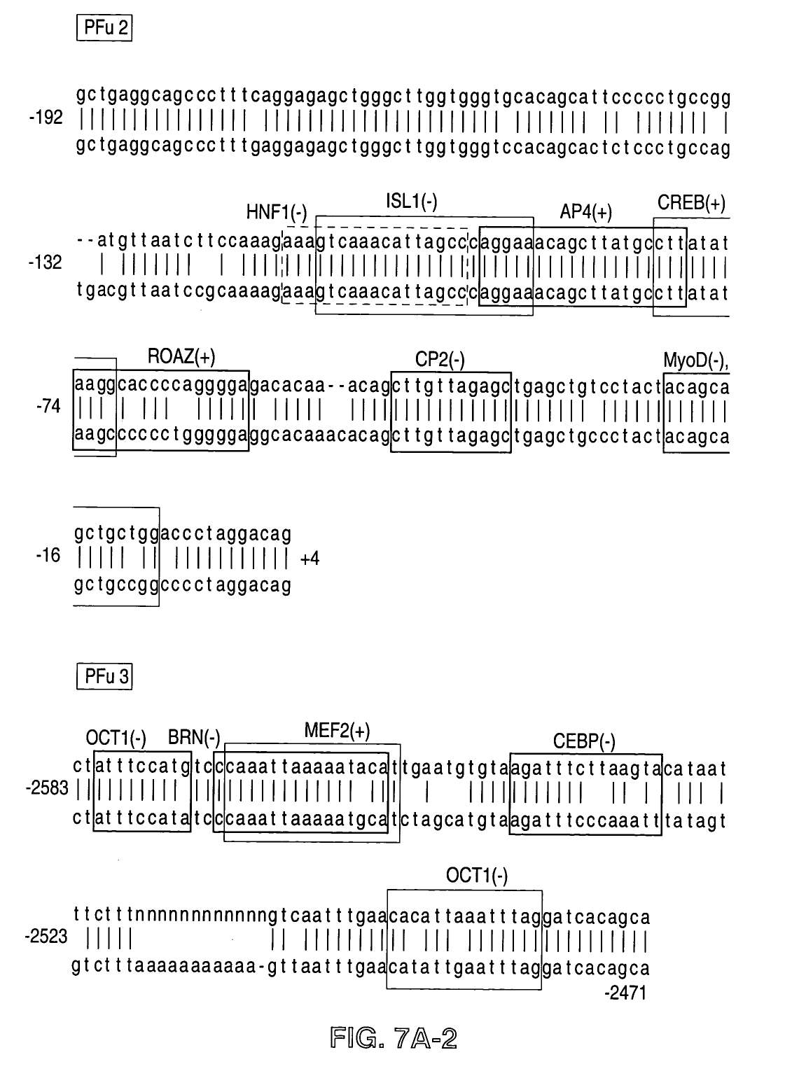

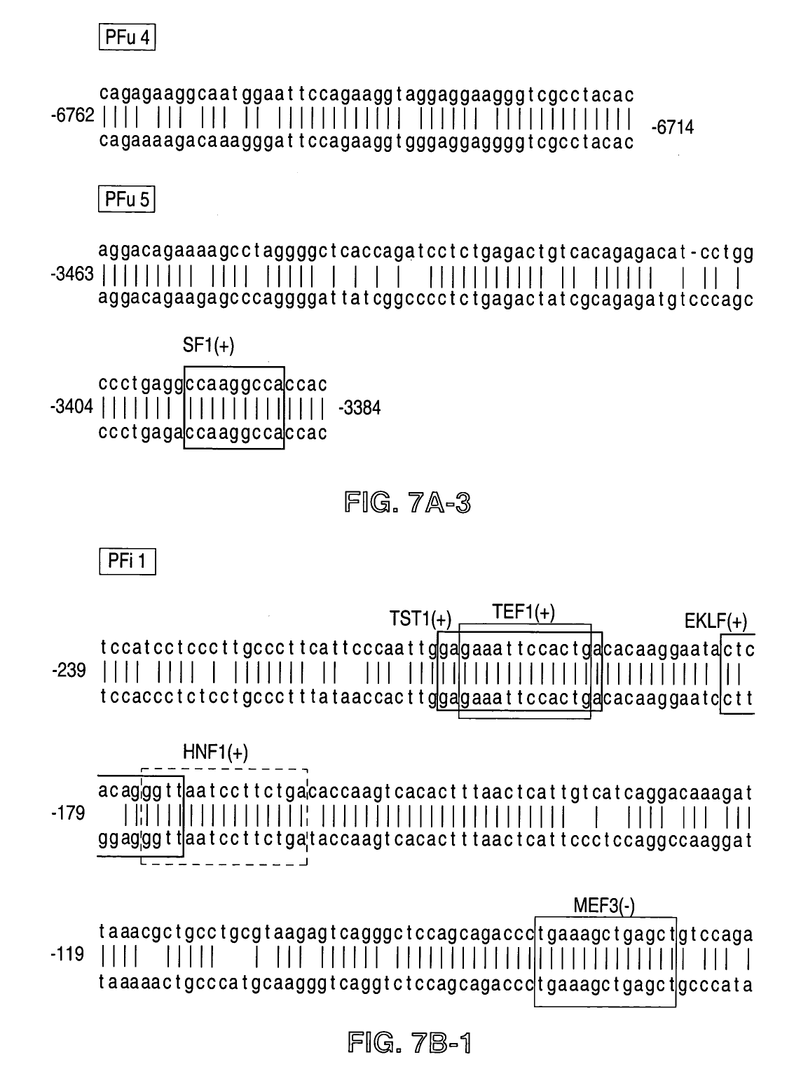

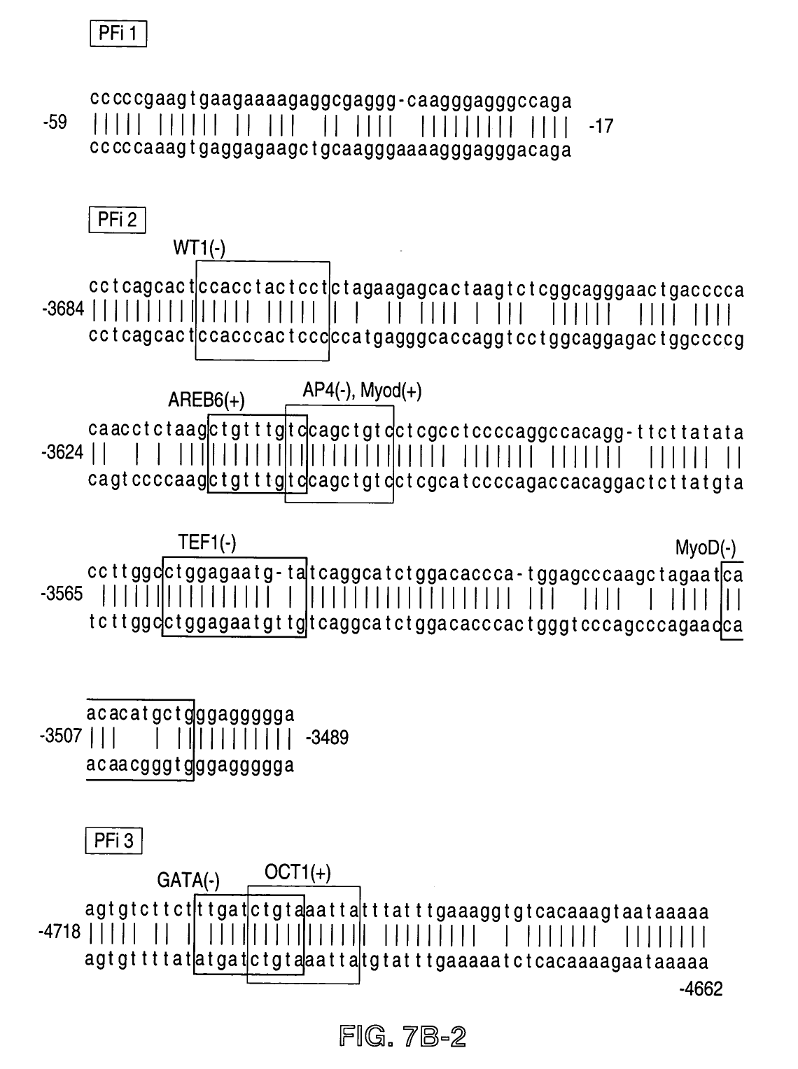

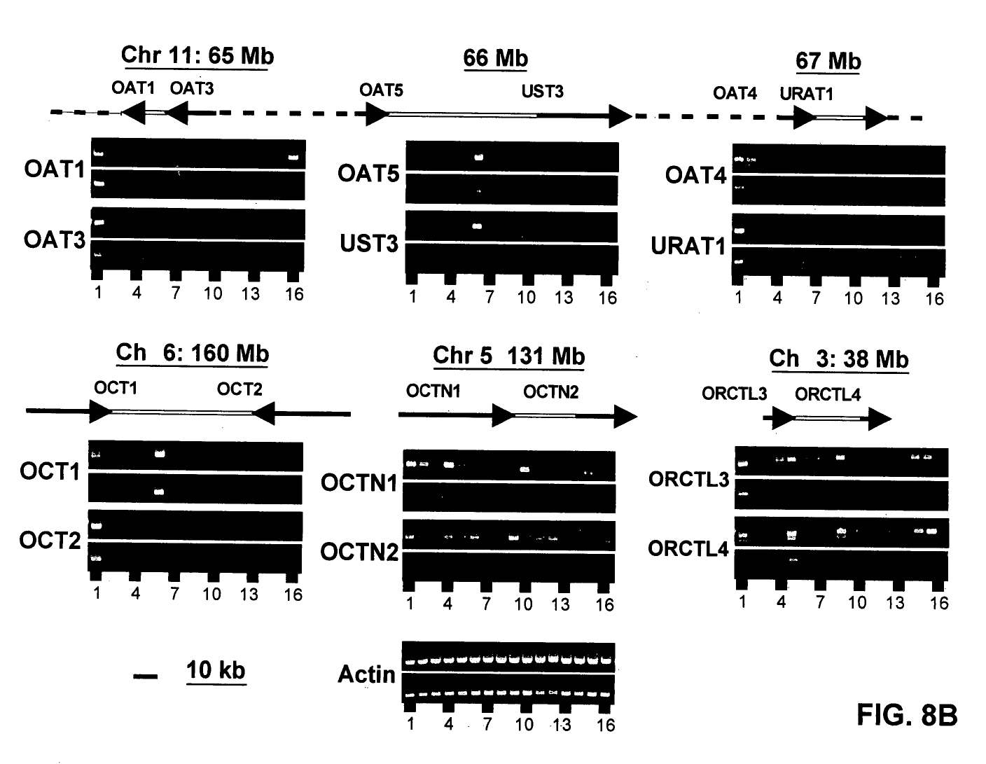

Over the last several years numerous transcription factor (TF) binding sites have been characterized leading to attempts to predict functional elements through identification of TF binding site matches within putative regulatory regions. However, due to the degeneracy of binding sites and the large size of mammalian genomes such searches have proved highly non-specific to the extent that the great majority of computationally identified TF sites have proved non-functional. Consequently multiple strategies have been advanced to improve specificity including prioritizing sites that are clustered occur multiply or are biologically plausible. In the methods of the invention, it was reasoned that only retaining TF sites that fell within PFs (using the PFs as a “filter” as it were to separate relevant from irrelevant sites) would greatly increase the likelihood of identifying functional sites. Matches were retained only if present in both mouse and human sequences; i.e. TF sites were required to both occur in a generally conserved region (the PF) as well as to themselves being specifically conserved. Numerous conserved motifs were identified (boxed in FIG. 7) many of which promisingly recognize factors of demonstrated importance in the differentiation of the kidney the major site of expression of OAT1 and 3 in adult (FIG. 8B). These include PAX1 PBX WT1 (Wilms' tumor suppressor) TCF and HNF1 and are indicated by gray boxes. Among these HNF1 is a particularly plausible candidate for a role in the transcriptional regulation of OAT1 and 3 as it has been demonstrated to induce transcription of other renal transporters including the Na-phosphate cotransporter (NaPi) and the Type II Na-glucose cotransporter (SGLT2). These regulatory functions likely account in part for the finding that HNF1 knock-out mice are a model of Fanconi syndrome-proximal tubular dysfunction resulting in urinary loss of glucose amino-acids and phosphates. One might therefore predict that these knockout mice manifest defective renal excretion of organic anions.

In another aspect, the invention provides a novel OAT termed OAT6. OAT6 is expressed predominantly in olfactory mucosa and testis. A sequence comparison and intron phasing analysis indicate that OAT6 is closely related to OAT1 and OAT3. OAT6 is also primal to he OAT1/OAT3 gene pair. Embryonic expression was observed at day 7. The data obtained from OAT6 suggest that olfactory mucosa may have a significant transport apparatus which could be important in the design of new therapeutic approaches for direct nose-to-brain transfer of drugs and olfaction. Supporting this, the data demonstrate that OAT1, OCT1-2, and OCTN1-3 are also expressed in olfactory mucosa.

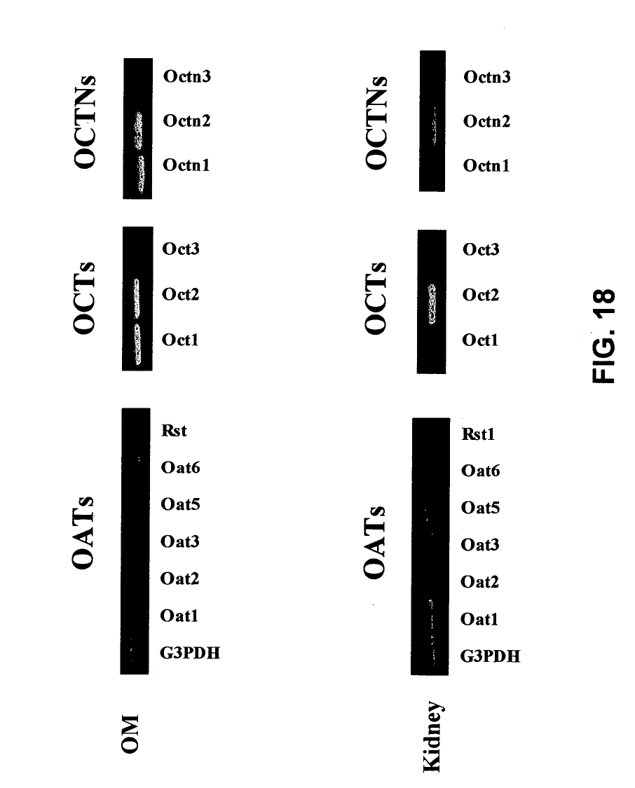

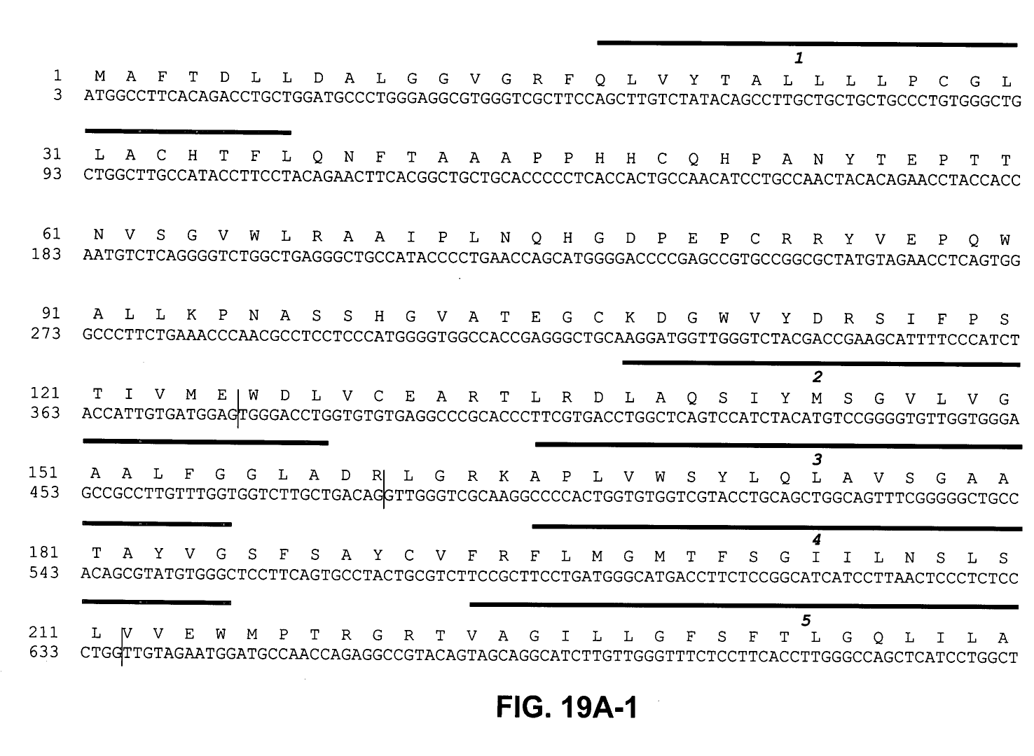

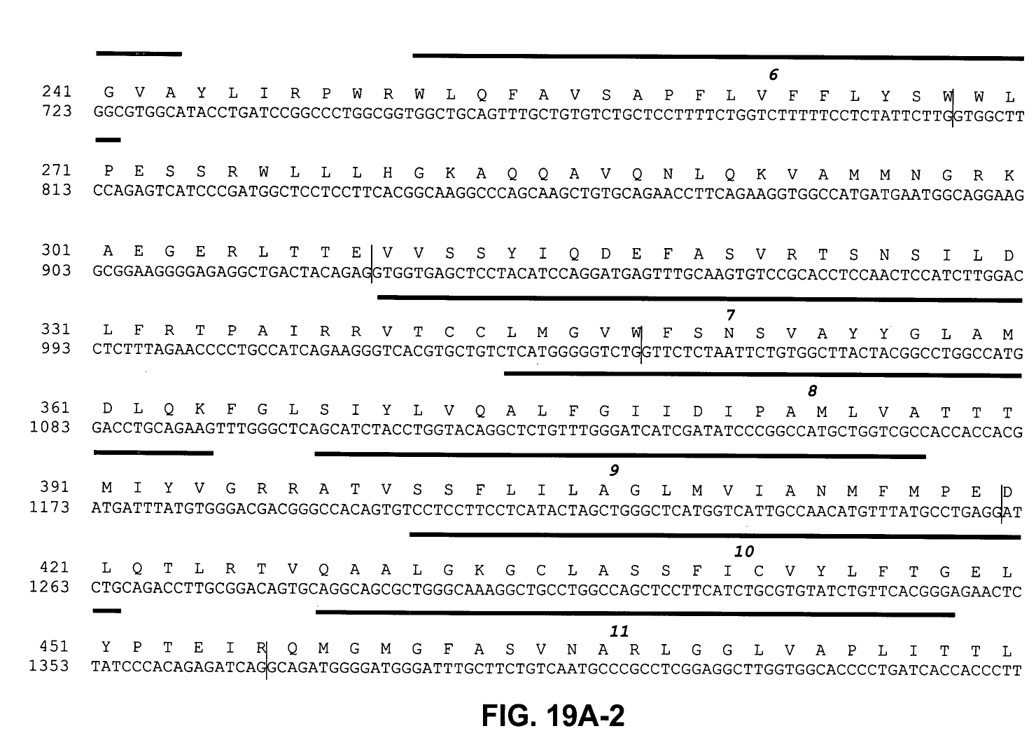

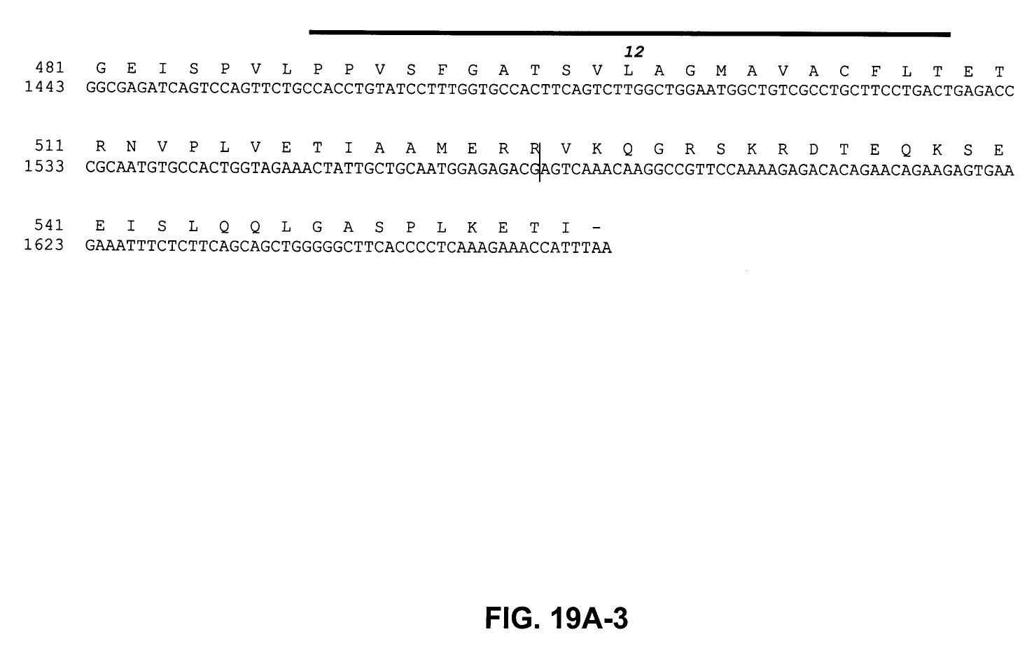

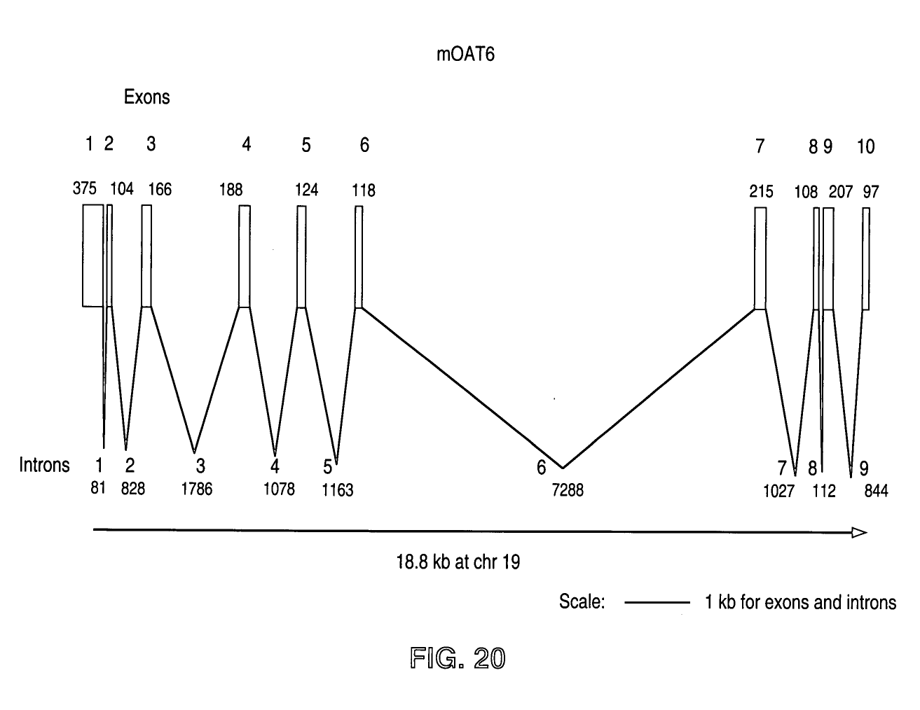

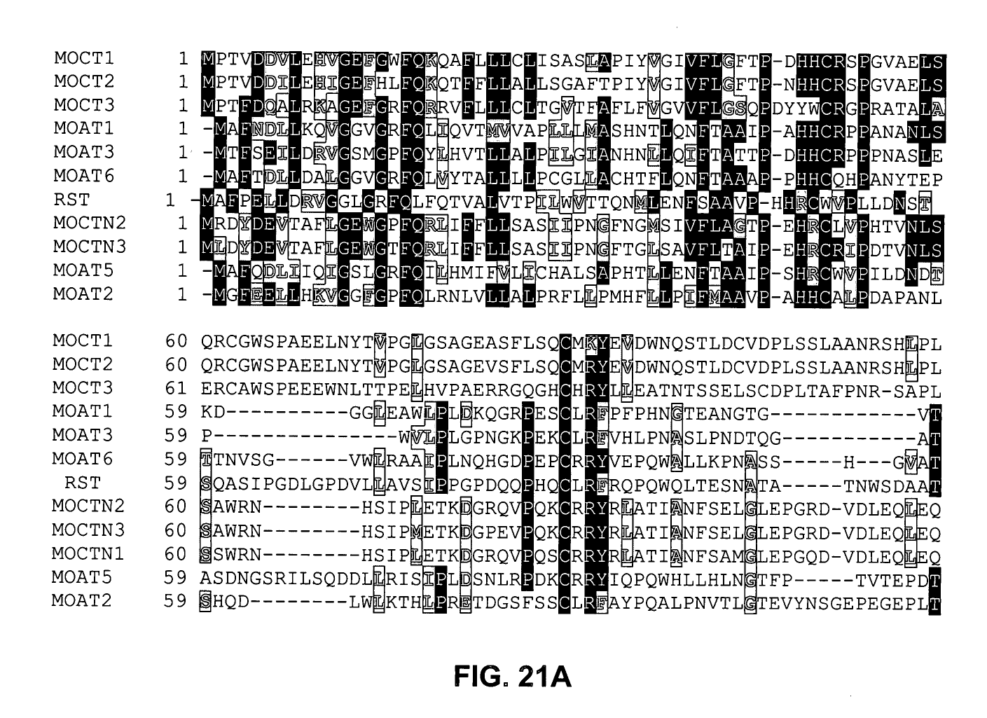

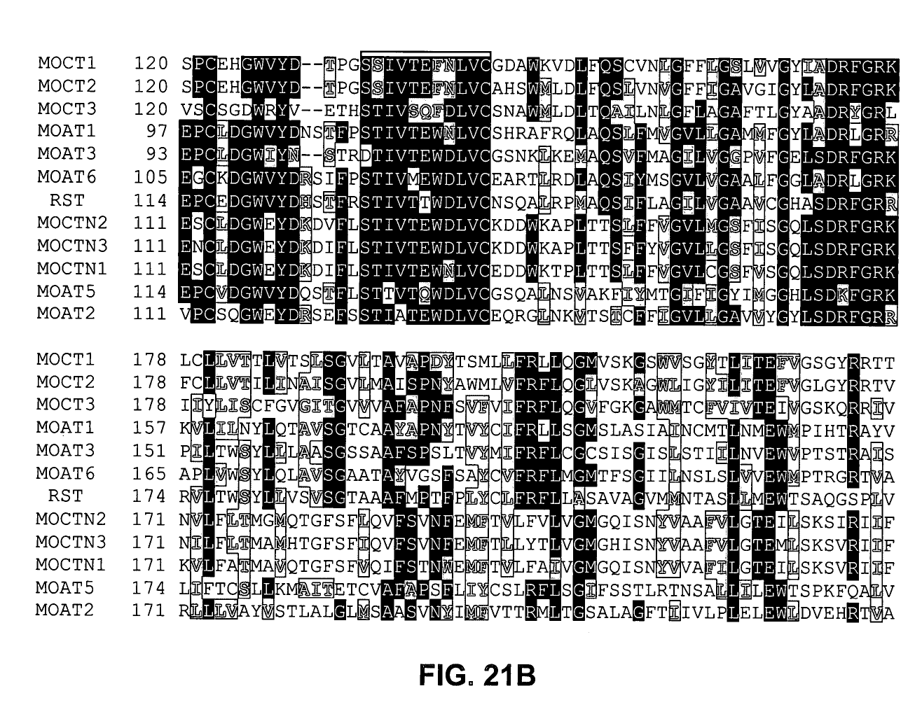

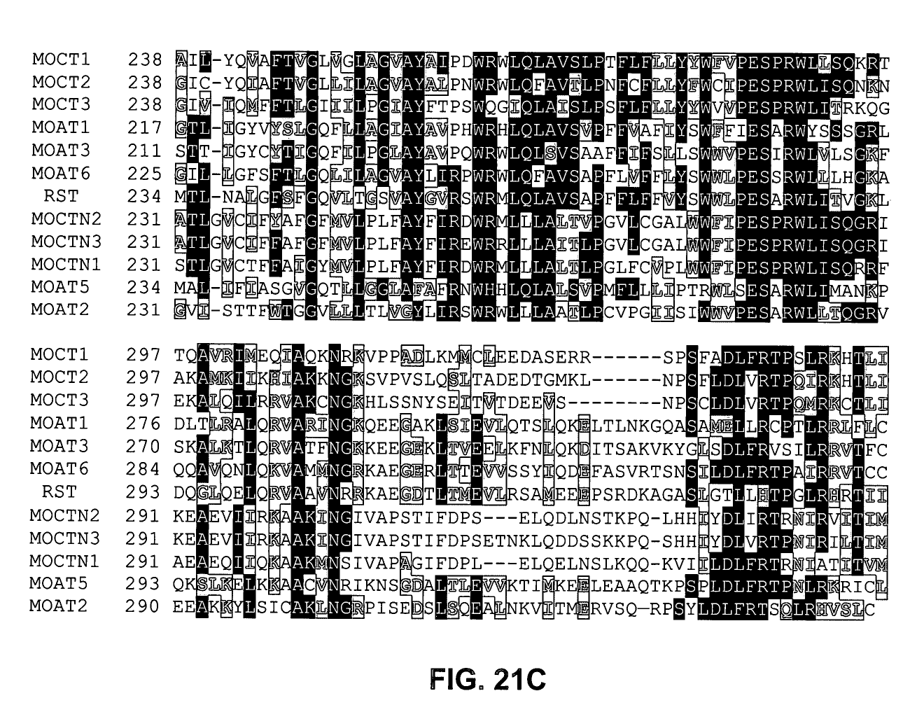

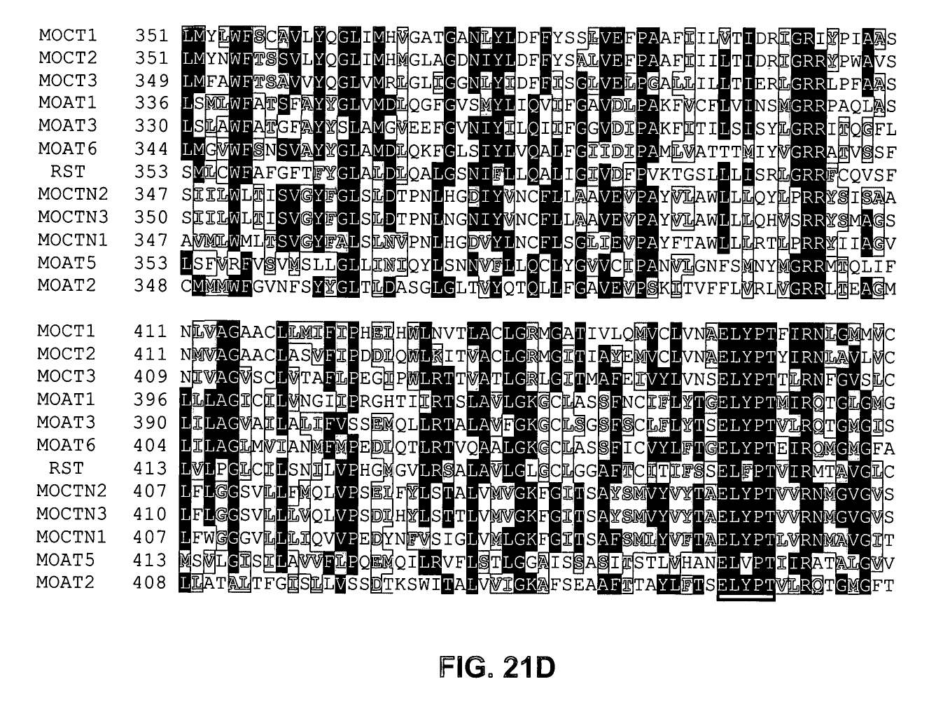

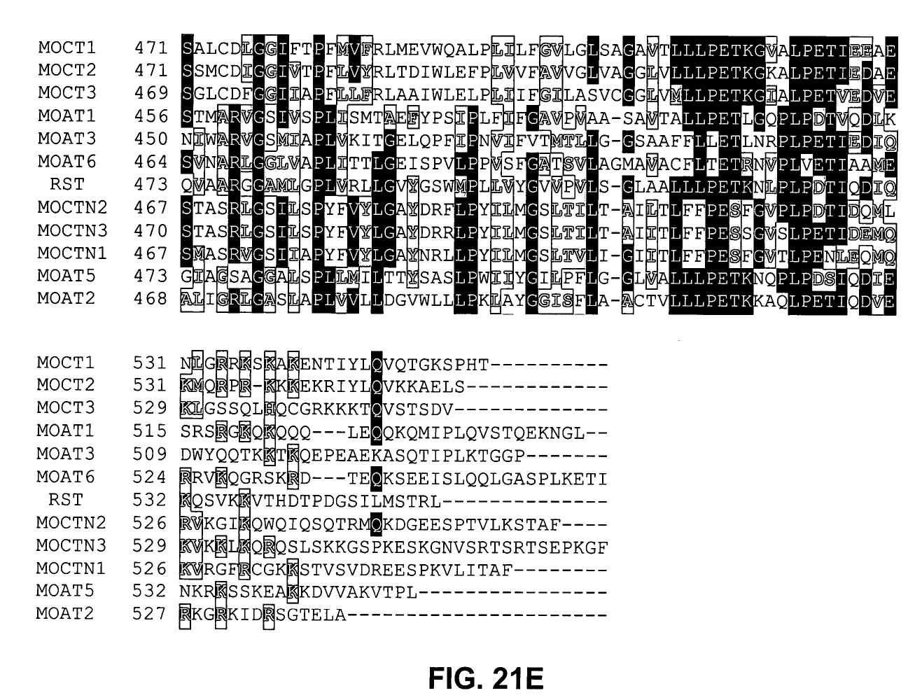

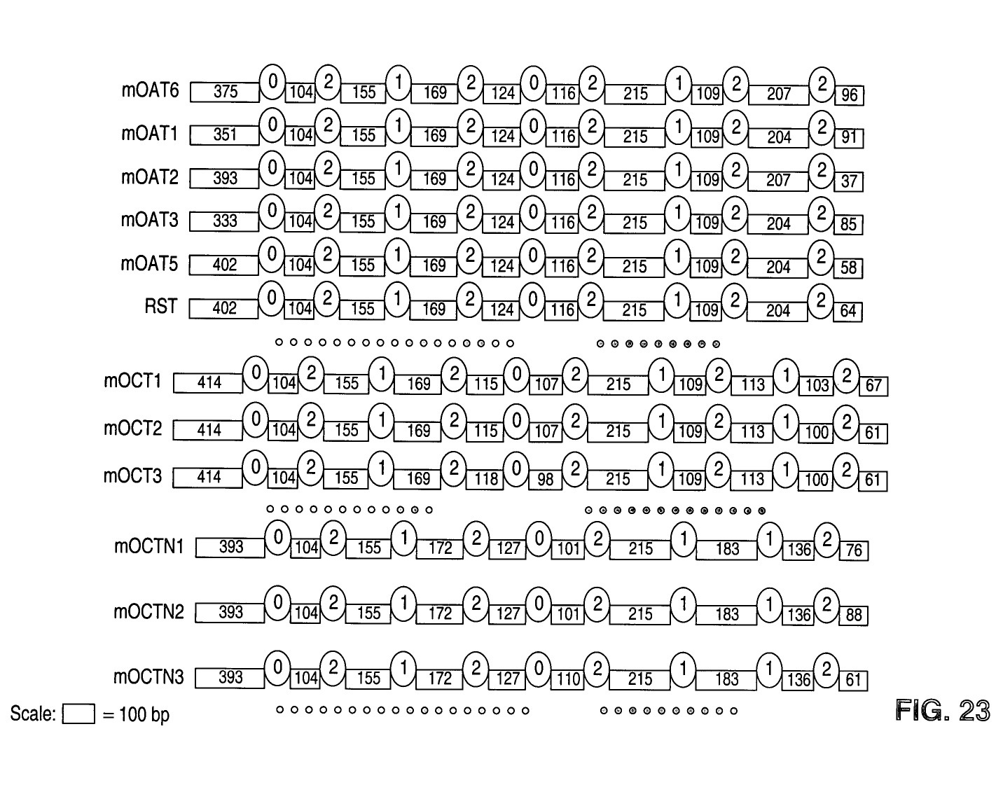

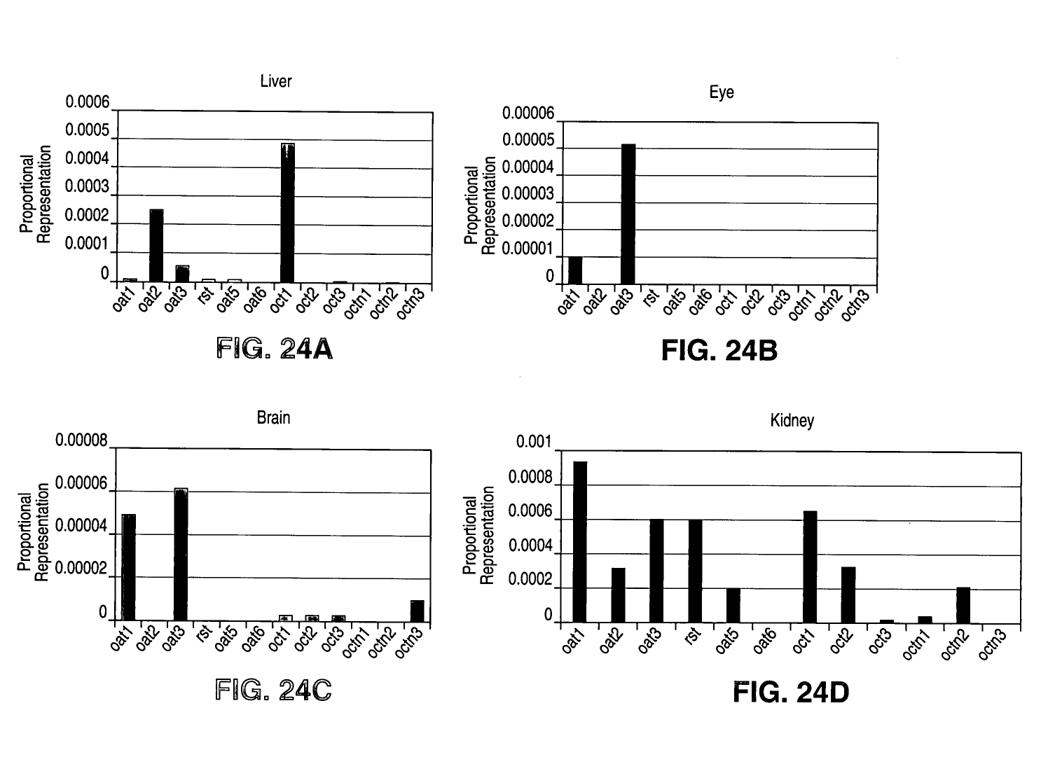

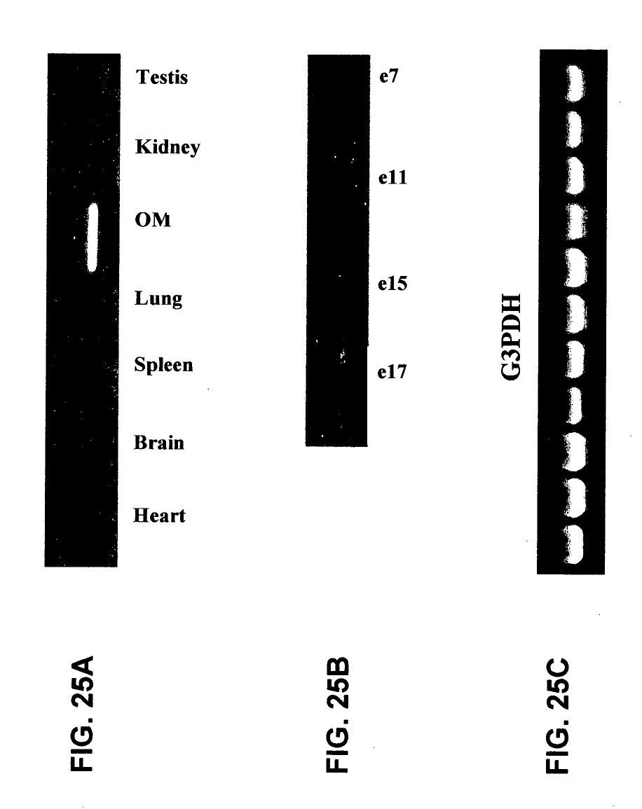

The invention thus provides a substantially purified OAT6 polypeptide. An OAT6 polypeptide sequence (SEQ ID NO:36) encoded by an OAT6 polynucleotide (SEQ ID NO:35) is shown in FIG. 18. An OAT6 polypeptide of the invention thus includes (i) a polypeptide comprising SEQ ID NO:36; (ii) a polypeptide encoded by a polynucleotide comprising SEQ ID NO:35; (iii) a polypeptide comprising a sequence that is at least 80%, 90%, 95%, 97%, 98% or 99% identical to SEQ ID NO:36 and has OAT6 activity; (iv) a polypeptide that is encoded by a polynucleotide that hybridizes to a nucleic acid consisting of SEQ ID NO:35 under moderate to high stringency conditions and wherein the polypeptide has OAT6 activity; and (v) a polypeptide comprising a fragment of any of (i) to (iv) above having OAT6 activity.

In one aspect, the OAT6 polypeptide may be altered by addition, substitution, or deletions of amino acids in order to modify its activity. For example, a peptide may be fused to the OAT6 polypeptide in order to effectuate additional enzymatic activity or to assist in purification or analysis. Alternatively, amino acids may be deleted to remove or modify the activity of the protein. Typically, deletions will be from 1 to 10 amino acids, 11-20 but typically less than 30% of the total number of amino acids in the OAT6 polypeptide. Useful fragments of OAT6 polypeptides comprise the extracellular, intracellular and/or soluble domains of OAT6. Such fragments are useful as antigens to generate antibodies.

In addition, an OAT6 polypeptide of the invention includes proteins or polypeptides that represent functionally equivalent polypeptides, for example and not by way of limitation, the sequences of SEQ ID NO:36 may contain deletions, additions or substitutions of amino acid residues within the polypeptide encoded by SEQ ID NO:35, but which results in a silent change, thus producing a functionally equivalent OAT6 polypeptide. Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; planar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent”, as utilized herein, refers to a polypeptide capable of exhibiting a substantially similar in vitro or in vivo activity as the endogenous OAT6 polypeptide encoded by the OAT6 polynucleotide described above, as judged by any of a number of criteria, including but not limited to antigenicity, i.e., the ability to bind to an anti-OAT6 antibody, immunogenicity, i.e., the ability to generate an antibody which is capable of binding a OAT6 protein or polypeptide, as well as molecular transport capabilities.

A substantially purified OAT6 protein, polypeptide, and derivative (including a fragment) is substantially free of other proteins, lipids, carbohydrates, nucleic acids, and other biological materials with which it is naturally associated. For example, a substantially purified functional fragments of OAT6 polypeptide can be at least 60%, by dry weight, the molecule of interest. One skilled in the art can purify functional fragment of OAT6 polypeptide using standard protein purification methods and the purity of the polypeptides can be determined using standard methods including, e.g., polyacrylamide gel electrophoresis (e.g., SDS-PAGE), column chromatography (e.g., high performance liquid chromatography), and amino-terminal amino acid sequence analysis.

Included within the scope of the invention are OAT6 proteins, polypeptides, and derivatives (including fragments) which are differentially modified during or after translation. Any of numerous chemical modifications may be carried out by known techniques, including but not limited to specific chemical cleavage by cyanogen bromide, trypsin, chymotrypsin, papain, V8 protease, acetylation, formylation, oxidation, reduction; metabolic synthesis in the presence of tunicamycin and the like. Additionally, the OAT6 polypeptide of the invention may be conjugated to other molecules to increase their water-solubility (e.g., polyethylene glycol), half-life, or ability to bind targeted tissue.

Furthermore, nonclassical amino acids or chemical amino acid analogs can be introduced as a substitution or addition into the OAT6 polypeptide. Non-classical amino acids include, but are not limited to, the D-isomer of the common amino acids, .alpha.-amino isobutyric acid, 4-aminobutyric acid, Abu, 2-amino butyric acid, .gamma.-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, beta-lanine, fluoroamino acids, designer amino acids, such as beta-methyl amino acids, alpha-methyl amino acids, N-alpha-methyl amino acids, and amino acid analogs in general. Furthermore, the amino acid can be D (dextrorotary) or L (levorotary).

While random mutations can be made to OAT6 polynucleotide (using random mutagenesis techniques known to those skilled in the art) and the resulting mutant OAT6 polypeptides tested for activity, site-directed mutation of the OAT6 polynucleotide can be engineered (using site-directed mutagenesis techniques well known to those skilled in the art) to create mutant OAT6 polypeptides with increased functional characteristics.

Peptides corresponding to one or more domains of the OAT6 polypeptide, truncated or deleted OAT6 proteins as well as fusion proteins in which the full length OAT6 proteins, polypeptides or derivatives (including fragments), or truncated OAT6, is fused to an unrelated protein are also within the scope of the invention and can be designed on the basis of the OAT6 nucleotide and OAT6 amino acid sequences disclosed herein. The fusion protein may also be engineered to contain a cleavage site located between a OAT6 polypeptide and the fusion domain, so that the OAT6 polypeptide may be cleaved away from the non-OAT6 moiety. Such fusion proteins or polypeptides include but are not limited to IgFc fusion which may stabilize the OAT6 protein in vivo; or fusion to an enzyme, fluorescent protein, or luminescent protein which provide a marker function.

The OAT6 polypeptide may be produced by recombinant DNA technology using techniques well known in the art. Method which are well known to those skilled in the art can be used to construct expression vectors containing an OAT6 polynucleotide and appropriate transcriptional translational control signals. These methods include, for example, in vitro recombinant DNA techniques, synthetic techniques, and in vivo genetic recombination. See, for example, the techniques described in Sambrook et al., 1989, supra, and Ausubel et al., 1989. Alternatively, RNA capable of encoding OAT6 polypeptide may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

As used herein, an “OAT6 polynucleotide” refers to (a) a polynucleotide comprising SEQ ID NO:35; (b) a polynucleotide that encodes a polypeptide having a sequence as set forth in SEQ ID NO:36 due to, for example, the degeneracy of the genetic code; (c) a polynucleotide that hybridizes to the complement of a nucleic acid consisting of SEQ ID NO:35, under, for example, stringent conditions, e.g., hybridization to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Willey & Sons, Inc., New York, at p. 2.10.3) and encodes a functionally equivalent OAT6 polypeptide; (d) a polynucleotide that hybridizes to the complement of a nucleic acid consisting of SEQ ID NO:35, under less stringent conditions, such as moderately stringent conditions, e.g., washing in 0.2% SSC/0.2% SDS/0.1% SDS at 42° C. (Ausubel et al., 1989, supra), and encodes a functionally equivalent OAT6 polypeptide; and (e) a fragment of any of (a) to (d) useful as primers, probes, encoding soluble domains, and/or antigenic fragments that are at least 15 nucleotides in length.

The invention also encompasses (a) vectors that contain any of the foregoing OAT6 polynucleotides including antisense molecules; (b) expression vectors that contain any of the foregoing OAT6 polynucleotide operatively associated with a regulatory element that directs the expression of the OAT6 polynucleotide; and (c) genetically engineered host cells that contain any of the foregoing OAT6 polynucleotides operatively associated with a regulatory element that directs the expression of the polynucleotide in the host cell. As used herein, regulatory elements include, but are not limited to, inducible and non-inducible promoters, enhancers, operators and other elements known to those skilled in the art that drive and regulate expression.

In addition to the gene sequences described above, homologs and orthologs of such OAT6 polypeptides and polynucleotides as may, for example, be present in other species, including humans, may be identified and may be readily isolated, without undue experimentation, by molecular biological techniques well known in the art. Further, there may exist genes at other genetic loci within the genome that encode proteins which have extensive homology to one or more domains of such gene products. These genes may also be identified via similar techniques.

The OAT6 gene and its homologs and orthologs can be obtained from other organisms thought to contain OAT6 activity. For obtaining cDNA, tissues and cells in which OAT6 is expressed are optimal. Tissues which can provide a source of genetic material for OAT6 and its homologs and orthologs, therefore, include testis and nasal mucosa from other species including humans.

For example, an isolated OAT6 polynucleotide may be labeled and used to screen a cDNA library constructed from mRNA obtained from the organism of interest. The hybridization conditions used should be of a lower stringency when the cDNA library is derived from an organism different from the type of organisms from which the labeled sequence was derived. Alternatively, the labeled fragment may be used to screen a genomic library derived from the organism of interest, again, using appropriately stringent condition. Low stringency conditions are well known in the art, and will vary predictably depending on the specific organism from which the library and the labeled sequences are derived. For guidance regarding such condition see, for example, Sambrook et al., 1989, Molecular Cloning, a Laboratory Manual, Cold Springs Harbor Press, N.Y.; and Ausubel et al., 1989, Current Protocols in Molecular Biology, Green Publishing Associates and Wiley Interscience, N.Y.

Further, a previously unknown OAT6 polynucleotide may be isolated by performing PCR using two degenerate oligonucleotide primer pools designed on the basis of the amino acid sequence of OAT6. The template for the reaction may be cDNA obtained by reverse transcription of mRNA prepared from human or non-human cell lines or tissue known or suspected to express an OAT6 gene.

An identified PCR product may be subcloned and sequenced to ensure that the amplified sequences represent the sequences of an OAT6 homolog or ortholog. The PCR fragment may then be used to isolate a full length cDNA clone by a variety of methods. For example, the amplified fragment may be labeled and used to screen a bacteriophage cDNA library. Alternatively, the labeled fragment may be used to screen a genomic library.

PCR technology may also be utilized to isolate full length cDNA sequences. For example, RNA may be isolated, following standard procedures, from an appropriate cellular or tissue source. A reverse transcription reaction may be performed on the RNA using an oligonucleotide primer specific for the most 5′ end of the amplified fragment for the priming of first strand synthesis. The resulting RNA/DNA hybrid may then be “tailed” with guanidines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies which may be used, see e.g., Sambrook et al., 1989, supra.

In cases where the OAT6 polynucleotide is the normal, or wild type nucleic acid, this polynucleotide may be used to isolate mutant alleles of OAT6. Mutant alleles may be isolated from subjects either known or proposed to have a genotype which contributes to drug toxicity and/or uptake difficulties of drug agents via nasal delivery. Mutant alleles and mutant allele products may then be utilized in the therapeutic and diagnostic systems described herein.

A cDNA of the mutant polynucleotide may be isolated, for example by PCR. In this case, the first cDNA strand may be synthesized by hybridizing an oligo-dT oligonucleotide to mRNA isolated from tissue known or suspected to be expressed in an individual putatively carrying the mutant allele, and by extending the new strand with reverse transcriptase. The second strand of the cDNA is then synthesized using an oligonucleotide that hybridizes specifically the 5′ end of the normal gene. Using these primers, the product is then amplified via PCR, cloned into a suitable vector, and subjected to DNA sequences analysis through methods known in the art. By comparing the DNA sequence of the mutant gene to that of the normal gene, the mutation(s) responsible for the loss or alteration of function of the mutant gene product can be ascertained.

A variety of host-expression vector systems may be utilized to express an OAT6 polynucleotide of the invention. Such host-expression systems represent vehicles by which the polynucleotide may be produced and subsequently purified, but also represent cells which, when transformed or transfected with the appropriate OAT6 polynucleotide, produce an OAT6 polypeptide of the invention. These include, but are not limited to, microorganisms such as bacteria (e.g., E. coli, B. subtilis) transformed with recombinant bacteriophage DNA, plasmid DNA or cosmid DNA expression vectors containing an OAT6 polynucleotide; yeast (e.g. Saccharomyces, Pichia) transformed with recombinant yeast expression vectors containing the OAT6 polynucleotide; insect cell systems infected with recombinant virus expression vectors (e.g., baculovirus) containing the OAT6 polynucleotide; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing an OAT6 polynucleotide; or mammalian cell systems (e.g., COS, SHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for an OAT6 polypeptide being expressed. For example, when a large quantity of such a polypeptide is to be produced, for the generation of pharmaceutical compositions of OAT6 polypeptide or for raising antibodies to an OAT6 polypeptide, for example, vectors which direct the expression of high levels of a fusion protein products that are readily purified may be desirable. Such vectors include, but are not limited to the E. coli expression vector pUR278 (Ruther et al., 1983, EMBO J. 2:1791), in which an OAT6 polynucleotide may be ligated individually into the vector in frame with the lac z coding region that a fusion protein is produced; pIN vectors (Inouye & Inouye, 1985, Nucleic Acids Res. 13:3101-3109); and the like. pGEX vectors may also be used to express foreign polypeptide as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa colifornica nuclear polyhedrosis virus (AcNPV) is used as a vector to express foreign genes. The virus grows in Spodoptera frugiperday cells. An OAT6 polynucleotide may be cloned individually into non-essential regions (for example the polyhedrin gene) of the virus and placed under the control of an AcNPV promoter. Successful insertion of an OAT6 polynucleotide will result in inactivation of the polyhedrin gene and production of non-occluded recombinant virus. These recombinant viruses are then used to infect S. frugiperda cells in which the inserted gene is expressed.

In mammalian host cells, a number of viral-based expression systems may be utilized. In cases where an adenovirus is used as an expression vector, an OAT6 polynucleotide may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing an OAT6 polypeptide in infected hosts (See Logan & Shenk, 1984, Proc. Nati. Acad. Sci, USA 81:3655-3659). Specific initiation signals may also be required for efficient translation of an inserted OAT6 polynucleotide. These signals include the ATG initiation codon and adjacent sequences. In cases where an OAT6 polynucleotide, including its own initiation codon and adjacent sequences, is inserted into the appropriate expression vector, no additional translation control signals may be needed. However, in cases where only a portion of an OAT6 polynucleotide is inserted, exogenous translational control signals, including, the ATG initiation codon must be provided.

Transfection via retroviral vectors, naked DNA methods and mechanical methods including micro injection and electroporation may be used to provide either stably transfected host cells (i.e., host cells that do not lose the exogenous DNA over time) or transient transfected host cells (i.e., host cells that lose the exogenous DNA during cell replication and growth).

The terms “identical” or percent “identity,” in the context of two or more nucleic acid molecules or polypeptide molecules, refer to two or more sequences or subsequences that are the same or have a specified percentage of nucleotides that are the same, when compared and aligned for maximum correspondence over a comparison window, as measured using a comparison algorithm or by manual alignment and visual inspection. Thus, if a sequence has the requisite sequence identity to the full sequence of SEQ ID NO:35 or 36 (polynucleotide or polypeptide, respectively) then it can also function to produce a polypeptide that has OAT6 activity (in the case of a polynucleotide) or is a functional OAT6 polypeptide (in the case of a polypeptide).

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identity for the test sequence(s) relative to the reference sequence, based on the designated or default program parameters. A “comparison window”, as used herein, includes reference to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Various algorithms are known in the art and include, e.g., the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, PILEUP, BESTFIT, FASTA, and TFASTA in the Wisconsin Genetics Software Package, Genetics Computer Group, 575 Science Dr., Madison, Wis.), or by manual alignment and visual inspection.

For purposes of determining percent sequence identity of the described invention (i.e., substantial similarity or identity) the BLAST algorithm is used, which is described in Altschul, J. Mol. Biol. 215:403-410, 1990. Software for performing BLAST analyses is publicly available through the National Center for Biotechnology Information (on the World Wide Web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. “T” is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues, always<0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment and include the following parameters for nucleotide comparison: a wordlength (W) of 11, an expectation (E) of 10, M=5, N=4. For amino acid sequences, the BLASTP program uses a wordlength (W) of 3, an expectation (E) of 10, and the BLOSUM62 scoring matrix (see, e.g., Henikoff, Proc. Natl. Acad. Sci. USA 89:10915, 1989).

The BLAST algorithm also performs a statistical analysis of the similarity between two sequences (see, e.g., Karlin, Proc. Nat'l. Acad. Sci. USA 90:5873-5787, 1993). One measure of similarity provided by the BLAST algorithm is the smallest sum-probability (P(N)), which provides an indication of the probability by which a match between two nucleotide or amino acid sequences would occur by chance. A nucleic acid is considered similar to a reference sequence if the smallest sum probability is less than 0.1. For example, it can be less than about 0.01, or less than about 0.001.

In another aspect, an OAT6 polypeptide can also be expressed in transgenic animals. Animals of any species, including, but not limited to, mice, rats, rabbits, guinea pigs, pigs, micro-pigs, goats, and non-human primates may be used to generate OAT6 transgenic animals.

Antibodies that interact with an OAT6 polypeptide are within the scope of this invention, and include antibodies capable of specifically recognizing one or more OAT6 polypeptide epitopes. Such antibodies may include, but are not limited to, polyclonal antibodies, monoclonal antibodies, humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Such antibodies may be used, for example, in the detection of an OAT6 polypeptide in a biological sample, including, but not limited to, mucosal tissue, testis tissue, blood, plasma, and serum. Alternatively, the antibodies may be used as a method for the inhibition of abnormal OAT6 polypeptide activity. Thus, such antibodies may be utilized as part of treatment for nasal mucosal disorders, and may be used as part of diagnostic techniques whereby subjects may be tested for abnormal levels of OAT6 polypeptides, or for the presence of abnormal forms of such polypeptides.

For the production of antibodies against an OAT6 polypeptide, various host animals may be immunized by injection with an OAT6 polypeptide, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsion, keyhole limpet hemocyanin, dinitrophenol, and potentially useful human adjuvants such as BCG, interferon and other cytokines effecting immunological response.

Polyclonal antibodies are a heterogenous population of antibody molecules derived from the sera of animals immunized with an antigen, such as an OAT6 polypeptide, or an antigenic functional derivative thereof. In general, for the production of polyclonal antibodies, host animals such as those described above, may be immunized by injection with .alpha.3-fucosyltransferase gene product supplemented with adjuvants as also described above.

Monoclonal antibodies (mAbs), which are homogenous population of antibodies to a particular antigen, may be obtained by any technique which provides for the production of antibody molecules by continuous cell lines in culture. These techniques include, but are not limited to, the hybridoma technique of Kohler and Milstein, (1975, Nature 256:495-497; and U.S. Pat. No. 4,376,110), human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies against an OAT6 polypeptide. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity

The antibodies described above can be used in the detection of OAT6 polypeptides in biological samples. OAT6 polypeptide from blood or other tissue or cell type may be easily isolated using techniques which are well known to those of skill in the art. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

For example, antibodies, or fragments of antibodies, such as those described above, useful in the invention may be used to quantitatively or qualitatively detect the presence of wild type or mutant OAT6 polypeptides. This can be accomplished, for example, by immunofluorescence techniques employing a fluorescently labeled antibody coupled with light microscopic, flow cytometric, or fluorimetric detection. Such techniques are useful since the OAT6 polypeptides are expressed on the cell surface.

The antibodies (or fragments thereof) useful in the invention may, additionally, be employed histologically, as in immunofluorescence or immunoelectron microscopy, for in situ detection of OAT6 polypeptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the OAT6 polypeptide, but also its distribution in the examined tissue. Using the present invention, those skill in the art will readily perceive that any of a wide variety of histological methods (such as staining procedures) can be modified in order to achieve such in situ detection.

Immunoassays for wild type or mutant OAT6 polypeptides typically comprise incubating a biological sample, such as a biological fluid, including but not limited to blood, plasma, or blood serum, a tissue extract, freshly harvested cells, or cells which have been incubate in tissue culture, in the presence of a detectably labeled antibody capable of identifying OAT6 polypeptides, and detecting the bound antibody by any of a number of techniques well known in the art.

Detection may also be accomplished using any of a variety of other immunoassays. For example, by radioactively labeling the antibody or antibody fragments, it is possible to detect wild type or mutant OAT6 polypeptides through the use of radioimmunoassays (RIA) (see, for example, Weintraub, Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound such fluorescein isothiocyanate, rhodomine, phycoerythrin, phycocyanin, allophycocyanin and fluorescamine.

The antibody can also be detectably labeled using fluorescence emitting metals. Additionally the antibody may be detected by coupling it to a chemiluminescent compound such as luminol, isoluminol, theramatic acreidinium ester and oxalate ester.

Also provided are OAT6 knockout non-human animals. Using the sequence information provided herein, one of skill in the art can utilize the teachings described above for the creation of double-knockout organisms to generate OAT 6 knockout organisms. Such OAT6 knockout organism would be useful in identifying drug toxicity by inhalation (e.g., nasal delivery).

The following examples are provided to further demonstrate the invention and do not limit the disclosure or the claims.

### EXAMPLES

Transgenic Knockouts. To begin to develop in vivo model systems for the assessment of the contributions of specific OAT family members to detoxification, development, and disease, targeted disruption of the murine Oat3 gene was carried out. Surviving Oat3^{-/-} animals appear healthy, are fertile, and do not exhibit any gross morphological tissue abnormalities. No Oat3 mRNA expression was detected in kidney, liver, or choroid plexus (CP) of Oat3^{-/-} mice. A distinct phenotype manifested by a substantial loss of organic anion transport capacity in kidney and CP was identified. Uptake sensitive to inhibition by bromo-sulfophthalein or probenecid was observed for taurocholate, estrone sulfate, and para-aminohippurate in renal slices from wild-type mice, whereas in Oat3^{-/-} animals transport of these substances was greatly reduced. No discernable differences in uptake were observed between hepatic slices from wild-type and Oat3^{-/-} littermates, suggesting Oat3 does not play a major role in hepatic organic anion uptake. Cellular accumulation of fluorescein was reduced by about 75% in CP from Oat3^{-/-} mice. However, capillary accumulation of fluorescein-methotrexate was unchanged, indicating the effects of Oat3 loss are restricted to the entry step and that Oat3 is localized to the apical membrane of CP.

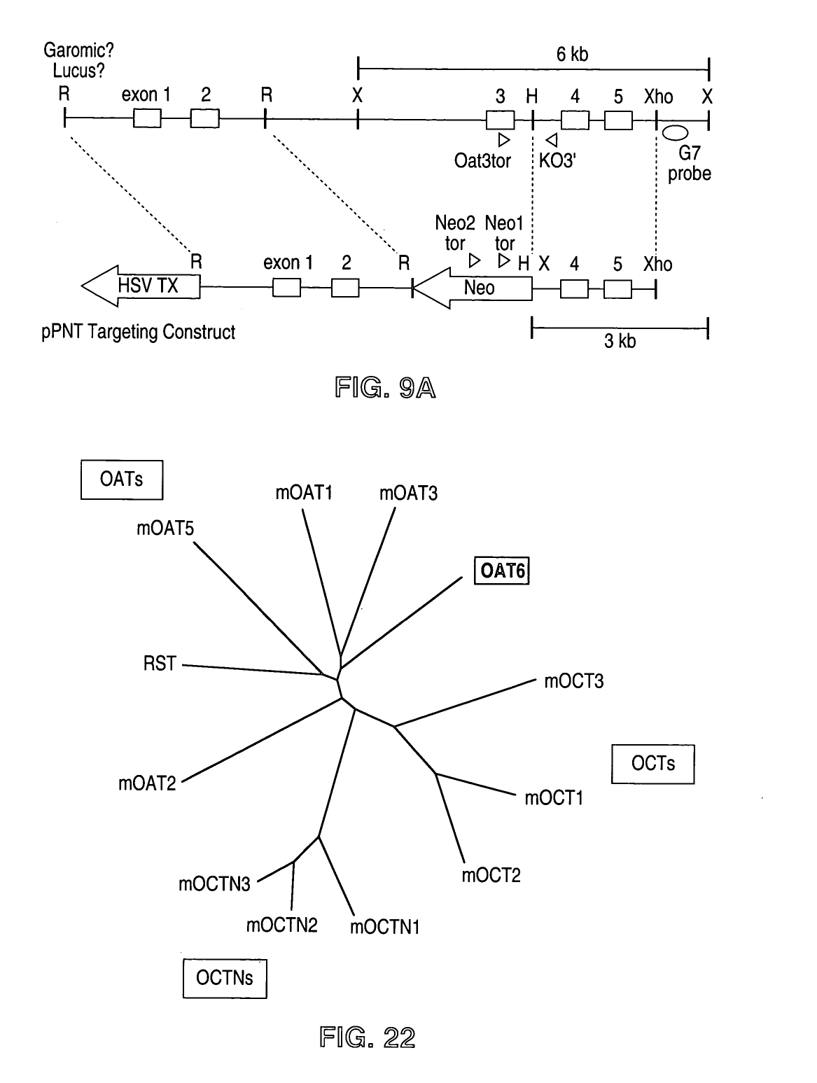

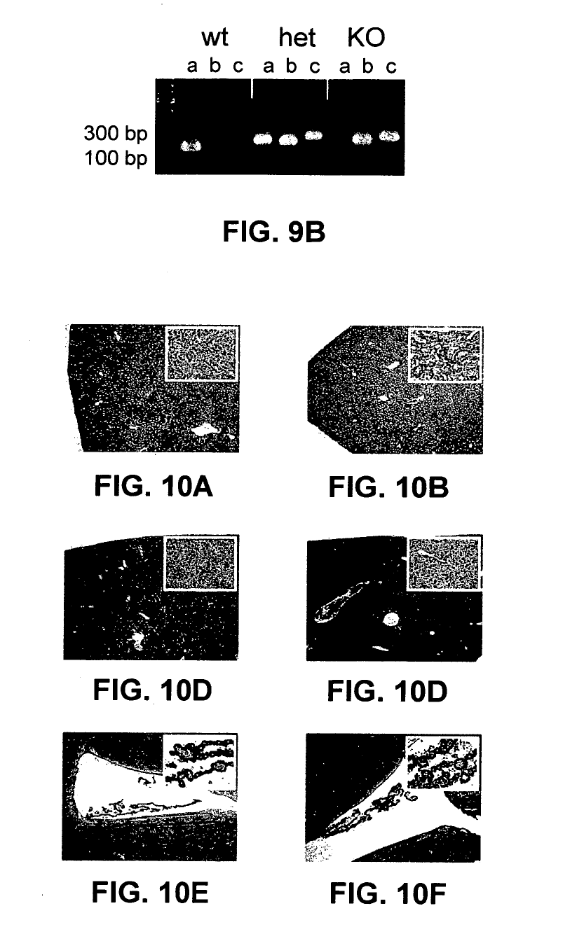

Oat3 Genomic Clone Isolation and Targeting Vector Construction. A BAC clone carrying the .Oat3 gene was isolated from the 129/Sv-derived CitbCJ7 library (Research Genetics, Inc., Huntsville, Ala.). A targeting construct for Oat3 was generated in the vector pPNT in which an internal fragment of the gene containing exon 3 was deleted and re-placed with a neomycin (Neo)-selectable marker (FIG. 9A). This was done by cloning a 6-kb EcoRI fragment containing exons 1 and 2 upstream of the Neo cassette (which is in an antisense orientation with respect to Oat3 transcription) and a 2-kb HindIII-XhoI fragment containing exons 4 and 5 downstream of the cassette. These fragments were inserted into pPNT such that the herpes simplex virus thymidine kinase cassette (used for counter selection) is upstream and in an antisense orientation with respect to the genomic sequences (FIG. 9A). Exon 3 deletion introduces a subsequent frameshift and premature stop codon such that direct splicing of exons 2 and 4 would result in a truncated peptide (281 versus 537 amino acids) with a scrambled amino acid sequence after residue 111.

Mice. The targeting construct was linearized by NotI digestion and electroporated into CJ-7 embryonic stem cells (a gift from Dr. Tom Gridley, Jackson Laboratory, Bar Harbor, Me.). Transfectants were selected in G418 (280 μg/ml) and ganciclovir (2 μM) and expanded for Southern blot analysis. Homologous recombinants were identified using the G7 probe, which is distal to the genomic sequences contained in the targeting construct (FIG. 9A). The G7 probe detects a 6-kb XbaI wild-type allele fragment and a 3-kb XbaI recombinant allele fragment. One embryonic stem cell line carrying both a wild-type and a targeted allele was identified in the first 35 clones analyzed; this was injected into blastocysts and a founder line established. Male chimeras were mated to C57BL/6 females, and heterozygous offspring were intercrossed to generate homozygous mutants.

A similar technique is carried out for a second OAT gene (e.g., OAT1) or an apical OAT (e.g., OAT4). The homozygotes knockouts for both OAT genes are then crossed to obtain double knockouts.

Mice were genotyped by polymerase chain reaction (PCR) analysis of their genomic DNA. Genomic DNA was isolated from tail snips by overnight digestion with 400 g/ml proteinase K in SNET buffer (20 mM Tris-Cl, pH 8, 5 mM EDTA, pH 8, 400 mM NaCl, and 1% w/v SDS) followed by extraction with phenol:chloroform:isoamyl alcohol and precipitation with isopropanol. Twenty nanograms of genomic DNA was used as template for PCR reactions using three different forward primers, one specific for exon 3 of the Oat3 gene (Oat3for) and two specific for the neomycin cassette present in the exon 3 deletion construct (Neolfor and Neo2for), each paired with a single reverse primer located in the intron region just prior to exon 4 of Oat3 (KO3): Oat3 for, 5′-CAGTCT-TCATGGCAGGTATACTGG-3′ (SEQ ID NO:1); Neolfor, 5′-GCGCATGCTCCAGACT-GCCTTGG-3′ (SEQ ID NO:2); Neo2for, 5′-GTGTAGCGCCAAGTGCCAGC-3′ (SEQ ID NO:3); KO3, 5′-GACAAAGAGAAGGCTATGACCTGG-3′ (SEQ ID NO:4). Cycle parameters were: denaturing at 95° C. for 15 min; followed by 30 cycles of 95° C. denaturing for 20 s, 60° C. annealing for 20 s, and 68° C. extension for 20 s. Homozygous wild-type mice do not carry Neo sequences and amplify only the Oat3for/KO3 combination. Mice homozygous for the targeted replacement of exon 3 with the inverted Neo cassette amplify only the Neolfor/KO3 and Neo2for/KO3 combinations. Heterozygous mice carry both alleles and amplify all 3 fragments. PCR products for the Oat3for/KO3, Neol for/KO3, and Neo2for/KO3 primer pairs are 200, 200, and 230 bp, respectively, and were visualized on a 1% agarose gel stained with ethidium bromide.

Histopathological Analysis. Three wild-type and four Oat3 knockout animals were euthanized by CO₂ inhalation. Tissues were dissected into approximately 50 volumes of 10% buffered formalin and fixed for 3 days prior to paraffin embedding. Embedded tissue was sectioned, stained with hematoxylin and eosin, and examined by light microscopy.

Northern Analysis. Approximately 10 μg of total kidney and liver RNA from wild-type, heterozygous, and Oat3^{-/-} littermates was separated by electrophoresis on a 1% agarose formaldehyde gel in MOPS buffer, capillary transferred overnight to a charged nylon membrane (Osmonics, Westborough, Mass.) with 20×SSC, and UV-cross-linked at 20,000 J/cm² with a Stratalinker (Stratagene, La Jolla, Calif.). The blot was cut into identical halves with one probed for Oat1 gene expression and the other for Oat3 gene expression. The Oat1 probe template (a 1,368-bp rat Oat1 fragment from position 186 to 1554) was generated by PCR from a cDNA clone, and the full-length Oat3 probe template was generated by NotI-HindIII double digest of a cDNA clone. Both templates were gel-isolated prior to labeling using the Qiaquick Gel Extraction kit (Qiagen, Inc., Chatsworth, Calif.). The ³²P-labeled probes were generated by random prime labeling using the Rediprime II kit (Amersham Biosciences), hybridized overnight at 68° C. in QuickHyb hybridization buffer (Stratagene), and the blots washed under conditions of high stringency (0.1×SSC, 0.1% SDS). The blots were stripped in boiling 0.1% SDS and reprobed with human β-actin. The experiments were repeated with two independent sets of wild-type, heterozygous, and Oat3^{-/-} littermates. A blot containing total kidney and liver RNA from a male and a female wild-type mouse, a male Oat3 knockout mouse, and a male and a female wild-type rat was also prepared and screened as described herein.

RT-PCR analysis of CP. Total RNA was isolated from several freshly collected lateral CP from adult rat and wild-type and Oat3^{-/-} mice using the Absolutely RNA RT-PCR Miniprep kit (Stratagene) according to the manufacturer's protocols (including treatment with DNase I). After denaturation for 5 min at 70° C. in the presence of 0.5 μg of oligo(dT) primer (Invitrogen), CP RNA was reverse transcribed for 1 h at 42° C. with 200 units of Moloney murine leukemia virus reverse transcriptase (Promega, Madison, Wis.) in a 25 μl reaction (containing 25 units of RNasin and 0.5 mM amounts of each DNTP). One microliter of the reverse transcription reaction was used as template for subsequent PCR with the following intron-spanning Oat1, Oat2, and Oat3 gene-specific primer pairs: Oat1for: 5′-ATGCCTATCCACACCCGTGC-3′ (SEQ ID NO:5); Oat1rev, 5′-GGCAAAGCTAGTGGCAAACC-3′ (SEQ ID NO:6); Oat2fbr, 5′-GCTGCA-TGATGGTGTGGTTTGG-3′ (SEQ ID NO:7); Oat2rev, 5′-GTACAACTCGGACGTGAACAGG-3′ (SEQ ID NO:8); Oat3for, 5′-CAGTCTTCATGGCAGGTATACTGG-3′ (SEQ ID NO:9); Oat3rev, 5′-CTGTAGCCAGCGCCACTGAG-3′ (SEQ ID NO:10). Cycle parameters were: denaturing at 95° C. for 15 min; followed by 35 cycles of 95° C. denaturing for 20 s, 58° C. annealing for 20 s, and 68° C. extension for 20 s. Products were visualized on a 1% agarose gel. stained with ethidium bromide. Oat3 mRNA expression was detected in kidney of Oat3^{-/-} mice, but it was readily detected in wild-type and to a lesser degree in heterozygous littermates. No Oat3 signal was detected in liver. Oat1 gene expression was readily detected in the kidney, but not in the liver, of all three animals. The blots were stripped and reprobed with human β-actin to confirm the integrity of the RNA. The experiment was repeated in two independent sets of wild-type, heterozygous, and Oat3^{-/-} littermates with similar results. To examine sexual dimorphism of Oat3 expression in mice, a blot containing total kidney and liver RNA from a male (M) and a female (F) wild-type mouse, a male Oat3^{-/-} mouse, and a male and a female wild-type rat was prepared and screened. Oat3 expression was detected in the kidney of the male and female wild-type mice and rats. Importantly, a faint Oat3 signal was also detected in the male rat liver, but not in the liver of the male mouse. Inclusion of male Oat3 knockout RNA demonstrated specificity of the probes and screening of the blot for p-actin monitored sample integrity.

Transport Assays-Xenopus oocyte isolation procedures and uptake assay. Ovaries were removed from tricaine methanesulfonate anesthetized adult female Xenopus laevis and follicle-free stage V and stage VI oocytes were isolated by treatment with collagenase A. After an overnight recovery period in Barth's buffer at 18° C., oocytes were microinjected with 20 ng of capped cRNA synthesized from linearized cDNA (mMessage mMachine in vitro transcription kit, Ambion, Inc., Austin, Tex.). Three days after injection, oocytes were randomly divided into experimental groups (n of 5) and incubated for 1 h at room temperature in oocyte Ringer 2 (in mM: 82.5 NaCl, 2.5 KCl, 1 Na₂PO₄, 3 NaOH, 1 CaCl₂, 1 MgCl₂, 1 pyruvic acid, 5 HEPES, pH7.6) containing 10 μM [³H]para-aminohippurate (PAH, 1 pCi/ml), 90 nM [³H]estrone sulfate (ES, 1 μCi/ml), or 500 nM [³H]taurocholate (TC, 1 μCi/ml) in the absence or presence of 1 mM probenecid (Pro). Oocyte radioactivity was measured in disintegrations/min (dpm) in a Packard 1600TR liquid scintillation counter with external quench correction.

Renal and hepatic tissue slice preparation and uptake assays were performed according to standard protocols. Four- to 6-month-old mice were euthanized by CO₂ inhalation, and the liver and kidneys were immediately placed into freshly oxygenated ice-cold saline. Tissue slices (˜0.5 mm; ˜5-10 mg, wet weight) were cut with a Stadie-Riggs microtome and maintained in ice-cold modified Cross and Taggart saline (in mM: 95 NaCl, 80 mannitol, 5 KCl, 0.74 CaCl₂, and 9.5 Na₂PO₄, pH 7.4). Slices were incubated for 1 h with substrate (1 pM taurocholate or para-aminohippurate, 100 nM estrone sulfate, 10 μM tetraethylammonium (TEA)) in the presence and absence of inhibitors (1 mM bromo-sulfophthalein (BSP) or probenecid, 200 μM quinine sulfate (Q)). Conditions for the PAH experiments were optimized for Oat1 by the addition of 10 μM glutarate to the uptake buffer. After incubation the slices were removed from the uptake medium, blotted, weighed, dissolved in 1 ml of 1M NaOH, neutralized with 1 ml of 1M HCl, and assayed by liquid scintillation spectroscopy. Duplicate medium samples (50 μl) were also assayed, and data are presented as tissue to medium (T/M) ratios (i.e. dpm/mg of tissue divided by dpm/μl of medium).

Choroid plexus isolation. Adult male and female wild-type and Oat3^{-/-} mice were euthanized with CO₂. Lateral CP were dissected immediately and transferred to ice-cold artificial cerebrospinal fluid (aCSF (in mM): 103 NaCl, 4.7 KCl, 1.2 KH₂PO₄, 1.2 MgSO₄, 25 NaHCO₃, 2.5 CaCl₂, 10 glucose, and 1 sodium pyruvate, pH 7.4), previously gassed with 95% O₂, 5% CO₂. A forty-five min accumulation of 1 μM fluorescein (FL) or 2 μM fluorescein-methotrexate (FL-MTX) was measured in CP incubated in 1 ml of aCSF in Teflon incubation chambers maintained in Ziploc plastic bags containing 95% O₂, 5% CO₂ at room temperature until imaging.

Confocal Fluorescence Microscopy. CP were imaged as using an inverted Zeiss model 510 laser scanning confocal microscope fitted with a 40μ water immersion objective (numeric aperture, 1.2). Samples were illuminated with the 488-nm line of an argon laser; a 510-nm dichroic filter was in the light path, and a long pass emission filter (515 nm) was positioned in front of the detector. Single confocal images (512×512×8 bits; 4 frames line-averaged) were obtained and stored for later analysis. For FL and FL-MTX transport studies, cellular and capillary fluorescence intensities were measured from the stored confocal images using NIH ImageJ 1.25. For each CP, 5-10 adjacent cellular and capillary areas were selected. After background subtraction, the average pixel intensity for each area was calculated and the values reported graphically for each CP are the means ±S.E. for all selected areas (n˜5-10). Values reported in the text are mean ±S.E. of the individual mean values for each CP as determined above (n˜4-6 animals/group).

Statistics. The renal slice data were compared using unpaired Student's t-test. Differences in mean values between the control and inhibited conditions were considered significant when p≦0.05.

Chemicals. [³H]TC (2 Ci/mmol), [³H]ES (40 Ci/mmol), and [³H]PAH (4 Ci/mmol) were obtained from PerkinElmer Life Sciences. [¹⁴C]TEA (55 mCi/mmol) was obtained from American Radiolabeled Chemicals, Inc. (St. Louis, Mo.). Unlabeled TC, ES, PAH, TEA, BSP, Pro, and Q were obtained from Sigma. FL and FL-MTX were purchased from Molecular Probes (Eugene, Oreg.). All other chemicals were of reagent grade.

Using the methodology described above, exon 3 of the murine Oat3 gene, which corresponds to putative transmembrane domain 2 in the Oat3 protein, was replaced by an inverted neomycin cassette via homologous recombination in CJ-7 embryonic stem cells (FIG. 9A). Southern analysis of selected embryonic stem cell clones confirmed specific targeting of the Oat3a allele, and chimeric mice were generated by blastocyst injection. Homozygous Oat3^{-/-} mice from the F2 generation of chimeric Oat3 mice crossed with C57BL/6J animals were subsequently backcrossed 4 generations with the C57BL/6J strain. Offspring from heterozygous pairings were genotyped by PCR assay (FIG. 9B). Identified Oat3^{-/-} mice appear healthy and normal, do not exhibit shortened life expectancy as compared with wild-type littermates, and are fertile, and an Oat3 knockout colony has been established. Histological study of Oat3^{-/-} mice and wild-type littermates, with an emphasis on kidney, liver, and choroid plexus, did not reveal any gross morphological abnormalities (FIG. 10).

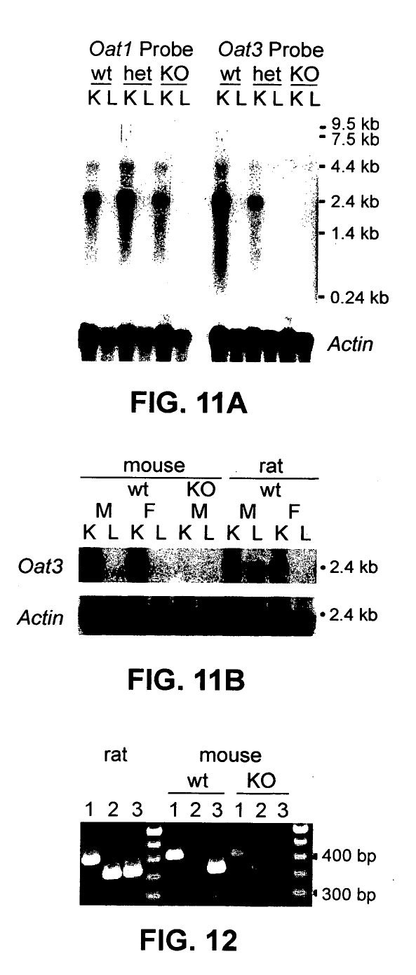

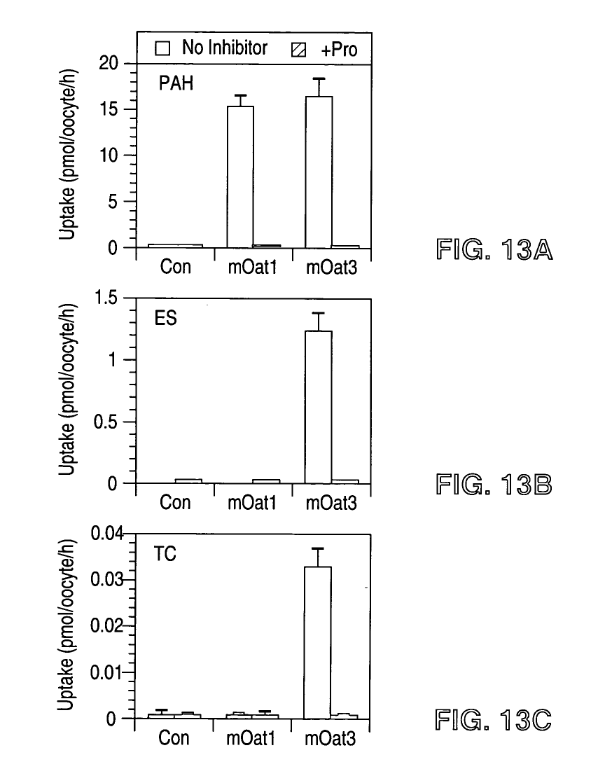

No Oat3 mRNA expression was detected in the kidney of Oat3^{-/-} mice by Northern analysis, but an approx 2.2-2.4 kb band corresponding to Oat3 was readily detected in wild-type littermates and to a lesser degree in heterozygous Oat3± mice (FIG. 11A). No Oat3 signal was observed in the liver. The blot was stripped and re-exposed to a human beta actin probe to confirm the integrity of RNA transferred to the blot (FIG. 11A). The experiment was repeated in a second set of littermates and yielded similar results. Expression of Oat1, a gene known to be expressed exclusively in the kidney and choroid plexus of adult rats, was also examined. In both sets of animals, Oat1 gene expression was detected in the kidney, but not in the liver, of wild-type, Oat3^{±}, and Oat3^{-/-} littermates (FIG. 11A). Differences in Oat3 expression between male and female wild-type mice and rats were also examined (FIG. 11B). Screening of the blot for beta actin confirmed sample integrity.

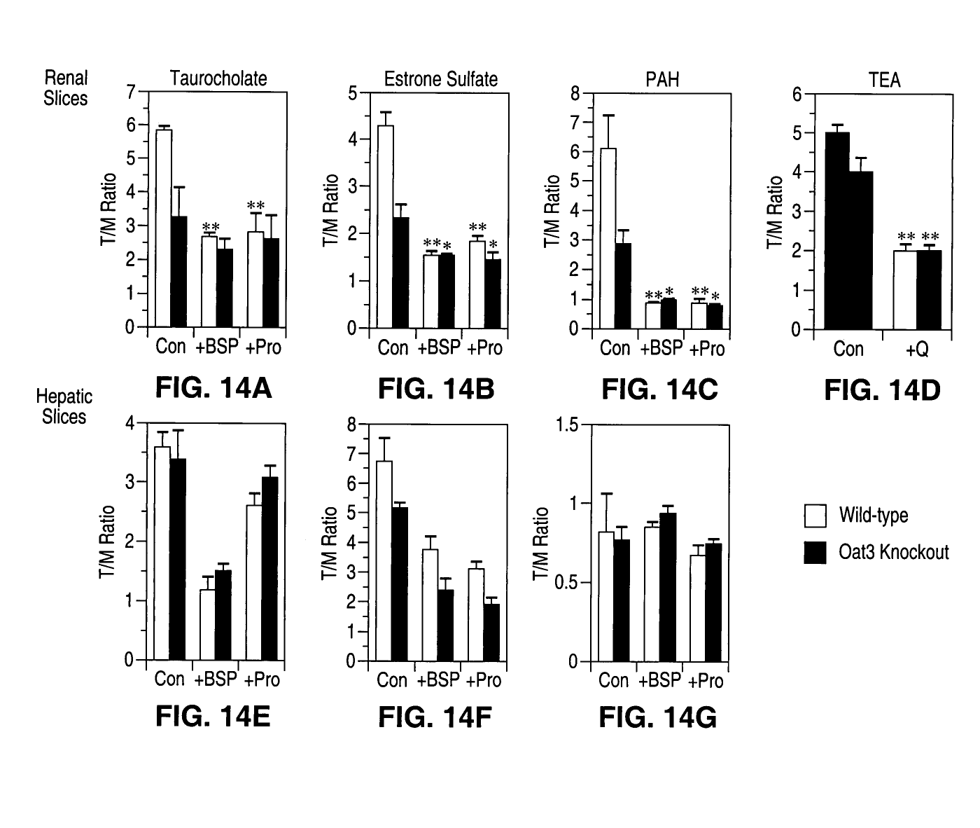

The significant (p<0.05) drop in TC uptake combined with the lack of any inhibitory indicates that renal taurocholate uptake is largely mediated by Oat3 and that Oat3^{-/-} mice have a demonstrable OA-deficient transport phenotype (FIG. 14). The significant (p<0.05) reduction in estrone sulfate transport in Oat3^{-/-} mice also supports this interpretation, with the additional drop in ES transport in the presence of BSP and Pro potentially because of Oat4 expression in the basolateral membrane of proximal tubule cells (FIG. 14). Although there is a significant (p<0.001) decrease in PAH uptake associated with Oat3 loss, there is nonetheless a large inhibitor-sensitive transport component left in renal slices from knockout animals, presumably representative of intact Oat1 transport function. The residual Oat4-mediated ES uptake and Oat1-mediated PAH uptake, along with unaltered organic cation (TEA) transport, in Oat3^{-/-} renal slices confirms that the observed OA transport-deficient phenotype in these animals is the result of specific Oat3 loss, as opposed to a generalized, nondescript disruption of transport function.

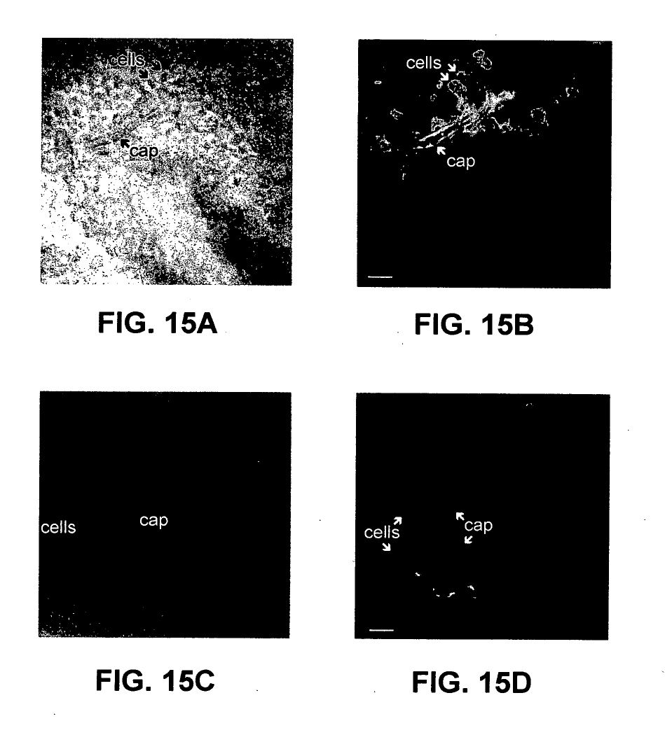

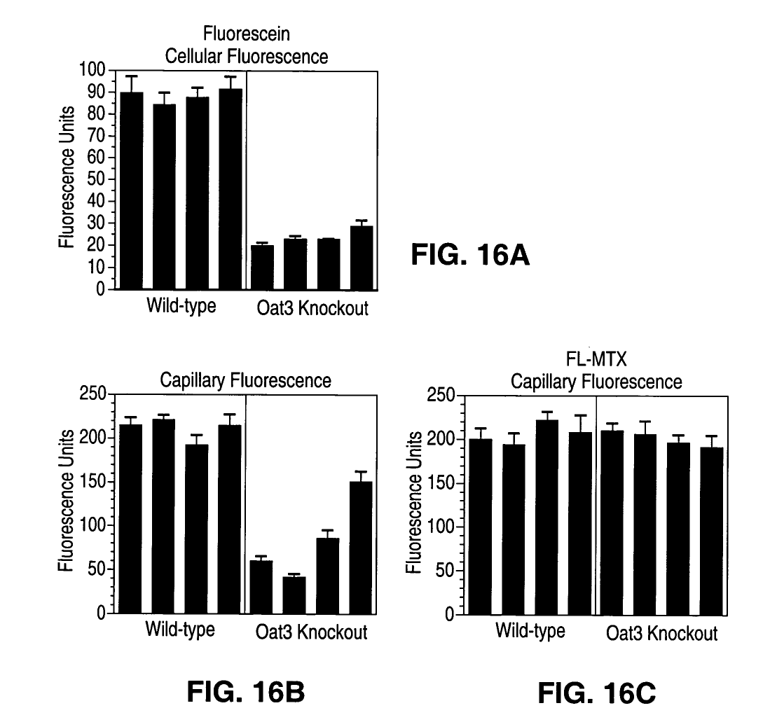

Recently it was reported that Oat3 is also expressed in the liver (In male, but not female, rats); however, in these studies no OAT3 expression was detected in mouse liver RNA from wild-type or heterozygous Oat3^{±} male mice (FIGS. 3, A and B). Regardless, to avoid the possibility that using female wild-type mice as control animals in hepatic transport studies would mask any actual change in OA transport as a result of Oat3 loss, only data using male wild-type littermates are presented in FIG. 14. Therefore, basolaterally expressed Oat2 would be the only OAT present in liver and none of the compounds used in this study are known substrates for Oat2. Thus, all of the uptake measured in hepatic slices should be attributable to non-OAT transporters. As such, Oatp1-4 have all been detected in liver and Oatp1, Oatp2, and Oatp4 have been localized to the basolateral membrane by immunocytochemistry. This interpretation is further supported by the lack of any significant difference in uptake between wild-type and Oat3^{-/-} mice (FIG. 14) for taurocholate and estrone sulfate and the complete lack of hepatic PAH uptake (PAH is not a substrate for Oat2 or Oatp1-4). As indicated in FIG. 12, OAT1, Oat2, and Oat3 expression has been detected in rat and murine CP. In rat CP, apical uptake of the organic anions PAH, 2,4-dichlorophenoxyacetic acid, and FL has been demonstrated to occur at least in part via the indirect sodium-coupled exchange mechanism utilized by Oat1. Furthermore, OAT1 and Na⁺, K⁺-ATPase have been demonstrated to be targeted to the apical membrane in rat CP. Thus, the CP is unique in that, to accomplish the extraction of organic anions (OA) from CSF to blood, the tissue exhibits a reversal of functional polarity as compared with other excretory epithelia (e.g. kidney and liver). Here, the disruption of the Oat3 gene leads to a significant decrease in FL uptake by murine choroid plexus, suggesting that Oat3, too, is involved in OA transport across the apical membrane of the CP (FIGS. 15 and 16). This is the first demonstration that Oat3 is localized to the apical surface (CSF side) of CP. The residual probenecid-sensitive FL uptake observed in CP from Oat3 knockout animals is presumably the result of functional OAT1 and, perhaps, Oat2. Thus, the OATs are poised to play an active role in the regulation of the composition of the extracellular fluid of the central nervous system compartment and in the protection of the central nervous system from toxic injury by mediating the selective exchange of OA substrates. Importantly, Oatp1 and Oatp: have also been detected specifically in CP, with Oatp1 immunolocalized to the apical membrane and Oatp2 to the basal membrane, whereas the expression of Oatp3 in brain is under dispute. Therefore, for the Oatps, currently only Oatp1 would be positioned to contribute to apical.

However, it has been demonstrated that FL, although a good substrate for OAT1 and Oat3, does not inhibit BSP uptake mediated by Oatp1 indicating that Oatp1 is probably not involved in apical FL uptake in the CP. Thus, the loss of FL transport noted in CP from Oat3^{-/-} animals in this study can be attributed to loss of Oat3 function. Observation of FL-MTX fluorescence levels in the underlying capillaries of the choroidal epithelium allows direct examination of one exit step across the basolateral membrane of the CP.

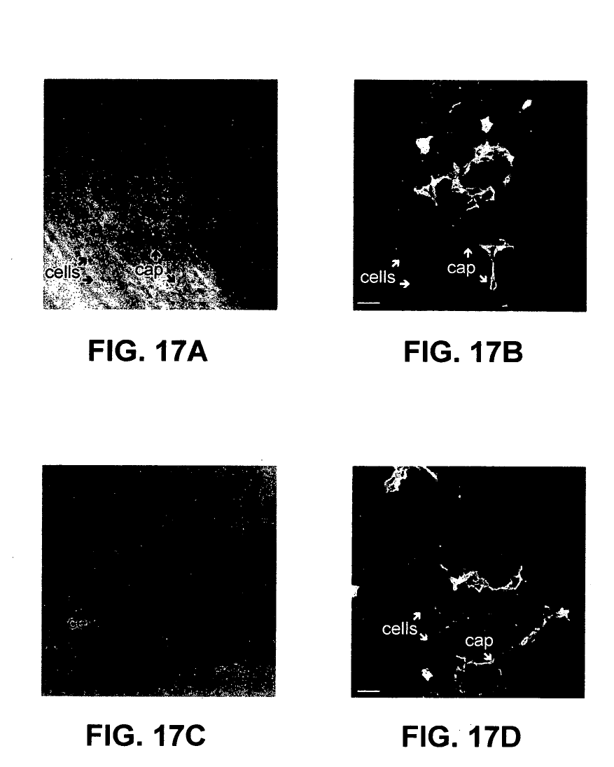

The fact that capillary accumulation of FL-MTX is unchanged between wild-type and Oat3^{-/-} CP (FIGS. 16 and 17) demonstrates that the basolateral exit step is unaffected by Oat3 loss, regardless of the transporter(s)responsible. This confirms that other uptake and eMux transporters are functional in Oat3^{-/-} CP and, in turn, corroborates the supposition that the marked reduction (˜75%) in cellular fluorescence observed for FL uptake in CP from Oat3 knockout animals is a result of decreased FL entry across the apical membrane.

Together, the data indicate an important role for Oat3 in the collective OA transport by kidney and CP. Particular substrates like taurocholate and estrone sulfate seem to be largely transported by Oat3, whereas it is likely that OAT1, and possibly Oat4, play equal or greater roles in the transport of other OATs. The results support the emerging model of OATs with overlapping specificities for a broad range of OA substrates, but high selectivity for certain substrates (e.g. TC and ES). It may be that OATs exhibit a type of affinity maturation for their substrates in that long term exposure results in expression or maturation of the more selective OAT. Thus, long term exposure of the knockout mice to certain OAT substrates may lead to a phenotype or at least to altered expression of the remaining OATs as compared to wild type. The generation of knockout animals and their interbreeding will help to identify which sets of OATs are involved in the transport of particular endogenous substrates and drugs. They will also help to determine the influence of genetic background on kidney and CP (CSF to blood) transport, an issue with potential ramifications in humans. Although Oat3 and other OATs are expressed in non-renal, non-CP sites in developing tissues, no developmental defects were observed. The double knockouts of Oat3 and OAT1 will help to determine the role OATs play in organogenesis.

Genomic sequencing. BAC clones containing mouse OAT1 and OAT3 were identified by hybridization to flanking STS's. Clone 457111 from C57BL/6J library RPCI-23 was shotgun sequenced to approximately 5× coverage by standard methods. BAC DNA was sheared by sonication and the ends were repaired by T4 DNA polymerase and size-selected fragments were subcloned into the pGEM3 (Promega Madison Wis.). DNA from individual clones was amplified by PCR or affinity purified from alkaline lysates and sequenced using fluorescent dye terminator chemistries (Applied Bio-systems Foster City Calif.). Shotgun sequence fragments were assembled in Sequencher (Gene Codes Corporation) using a two-tiered stringency nucleation strategy to reduce false-positive assembly steps. This independent assembly is equivalent to both public and private assemblies of the same interval but contains fewer gaps. Assembled contigs were anchored and oriented relative to a dense map of BAC clones (approximately 5× coverage for the interval) by PCR-based STS content. Assemblies and contig assignments were further verified by colinearity of corresponding cDNA sequences available in GenBank.

Computational analyses. Sequences were filtered for repeats and phylogenetic footprints (PFs) were determined using pairwise BLAST (http://www.ncbi.nlm.nih.gov/BLAST/). PFs were selected that had “expect” values no greater than 10□ 3. The remaining less significant PFs all had “expect” values greater than 1. Thus there was not a continuous gradation in PF significance which might have rendered arbitrary any choice of significance threshold. (Similar results were obtained with PipMaker (http://io.cse.psu.edu/pipmaker/) using moderately stringent criteria of at least 70% identity over at least 70 bp.) Transcription factor (TF) binding site searches used MatInspector (http://www.genomatix.de/software) and the TransFac 6.0 data base (http://transfac.gbf.de/). The specificity of the searches was increased by confining them to PFs as well as only retaining matches found in both the human and mouse sequences. Thus, enabling a relatively relaxed criteria was employed (core similarity threshold of 0.75 matrix similarity threshold “optimized”). Genomic locations were determined by BLAST searches (http://genome.cse.ucsc.edu/) of the draft human or mouse genome or tBLASTn searches of the Fugu genome (www-aluminum.jgi-psf.org/). Peptide sequences of OATs and OCTs were obtained from GenBank (www-.nci.nlm.nih.gov) and were aligned with ClustalX (available at http://www-u-strasg.fr/BioInfo/ClustalX/Top.html) with the gap opening penalty set to 10 and the gap extension penalty set to 0.1. Dendrograms were generated from the ClustalX output using TreeView (available from tax-onomy.zoology.gla.ac.uk/). RT-PCR. Tissue distributions of various OATs and OCTs were determined by semi-quantitative RT-PCR. A panel of 16 human adult tissue cDNAs (source RNAs were pooled from multiple individuals in each case) were purchased from Clontech (Palo Alto Calif.). Serial dilutions were made from these cDNAs and 1 ng 10 pg and 100 fg of template used in PCRs. Primers were designed to span introns and amplify cDNA fragments of ˜400-600 bp. Primer sequences were the following: OAT1 forward: 5′-GAAGGAGCCAAATTGAGTATGG-3′ (SEQ ID NO:11); OAT1 reverse: 5′-TACAGGAAGATGCAGTTGAAGG-3′ (SEQ ID NO:12); OAT3 forward: 5′-CTATGGGTGTGGAAGAATTTGG-3′ (SEQ ID NO:13); OAT3 reverse: 5′-TCCCGTAAAGATGATATTGGGG-3′ (SEQ ID NO:14); OAT4 forward: 5′-CTGGGAAAGGGATGTTTTGG-3′ (SEQ ID NO:15); OAT4 reverse: 5′-ACCGTCTCGTTATTGGTTGG-3′ (SEQ ID NO:16); URAT1 forward: 5′-CTTTGGCTTCACCTTCTTCG-3′ (SEQ ID NO:17); URAT1 reverse: 5′-GTGGATTTTAGGACAGAGTTCC-3′ (SEQ ID NO:18); UST3 forward: 5′-ACCAGAGGAAGGCTTAAAGG-3′ (SEQ ID NO:19); UST3 reverse: 5′-GGTGGAGAATACACACTTAGG-3′ (SEQ ID NO:20); OAT5 forward: 5′-TGTAAGATCCACCATGCAGG-3′ (SEQ ID NO:21); OAT5 reverse: 5′-CCGTTAAGGTCATCAAGAGG-3′ (SEQ ID NO:22); OCT1 forward: 5′-GAACCTCTACCTGGATTTCC-3′ (SEQ ID NO:23); OCT1 reverse: 5′-TTCATGGTCTCTGGCAAAGC-3′ (SEQ ID NO:24); OCT2 forward: 5′-CTCATGCTTGGGAAGAATGG-3′ (SEQ ID NO:25); OCT2 reverse: 5′-CTCATGCTTGGGAAGAATGG-3′ (SEQ ID NO:26); OCTN1 forward: 5′-GTTACTTTGCTCTGTCTCTGG-3′ (SEQ ID NO:27); OCTN1 reverse: 5′-CATCTGCTCTAAGGTTTCTGG-3′ (SEQ ID NO:28); OCTN2 forward: 5′-TTGACCTGTGTCTGACTTGC-3′ (SEQ ID NO:29); OCTN2 reverse: 5′-GGAGCATTTATTATGAGCCTGG-3′ (SEQ ID NO:30); ORCTL3 forward: 5′-AAGACCAGCCTTGCTTATGG-3′ (SEQ ID NO:31); ORCTL3 reverse: 5′-TATGACCCAGTGACCTATGG-3′ (SEQ ID NO:32); and ORCTL4 forward: 5′-CAGAGCTGAAATCCATGACG-3′ (SEQ ID NO:33); ORCTL4 reverse: 5′-CATACTTGGCCACTCAATTCC-3′ (SEQ ID NO:34). Control human β-actin primers were provided by Clontech.

Amplifications were performed with the HotStart Taq kit (Qiagen Hilden Germany). Cycle parameters were denaturing at 95° C. for 15 min; followed y 35 cycles of 94° C. denaturing for 20 s 60° C annealing for 30 s and 72° C. extension for 30 s with the exception that amplifications of actin cDNA used an annealing temperature of 55° C. and were performed for only 30 cycles. PCR products were visualized on 1.5% agarose gels and stained with ethidium bromide.

Transcriptional Regulation of OAT clusters and OCT clusters. The genomic sequences of murine OAT1 and 3 is a first step in characterizing regulatory regions within OATs and OCTs. The invention provides a genomic sequence of murine OAT1 and 3 allowing for the delineation of the evolutionarily conserved regions within non-coding sequences so-called phylogenetic footprints (PFs) by comparison to the publicly available orthologous human sequences. Such PFs typically contain important regulatory elements explaining the selective pressure which has resulted in their conservation. As might be expected the specificity of PFs for the detection of such elements usually varies directly with evolutionary distance while their sensitivity varies inversely. Comparisons between human and mouse separated by approximately 100 million years allow for detection of potential regulatory elements with both good sensitivity and specificity and predictions based on such comparisons have been experimentally verified in several cases.

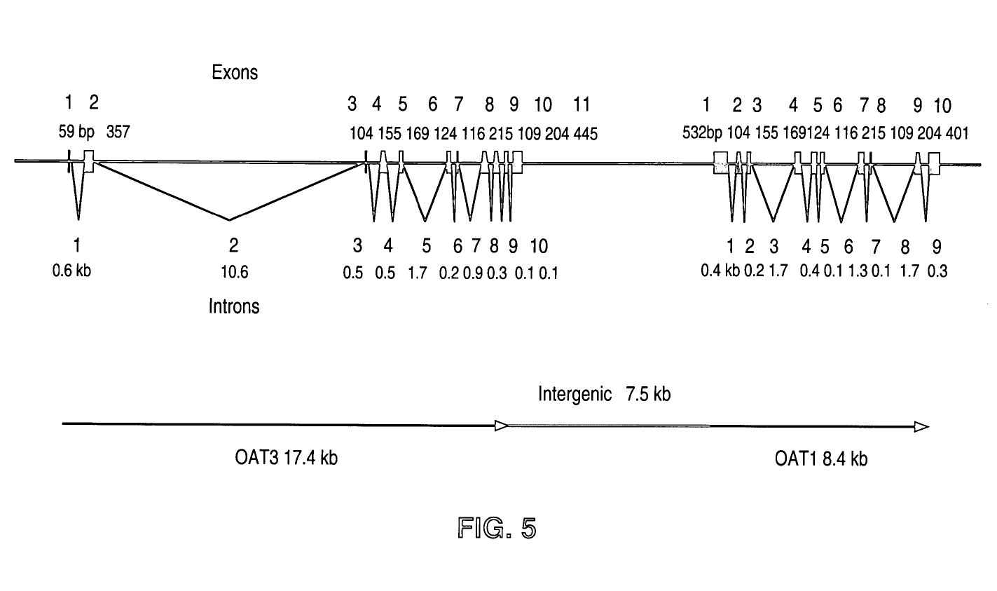

Murine OAT1 and 3 were located on a 59-kb BAC sequence derived from the region of mouse chromosome 19 syntenic to the human OAT1 and 3 locus at 11q12.3. Both the mouse and human genes are tightly linked with scant intergenic distances of 8.3 kb in human and 7.5 kb in mouse. The gene for OAT3 occupies 17.4 kb while that for OAT1 occupies 8.4 kb (FIG. 1). Thus including the 7.5-kb intergenic distance the two genes together occupy only 33.3 kb of mouse chromosome 19. Alignment of the cDNAs with the mouse genomic sequence reveals that OAT1 and 3 are composed of 10 and 11 exons respectively (FIG. 5). With the sole exception that OAT3 has an additional intron within its 5′ UTR. The structures of OAT1 and 3 are highly similar with other intron locations conserved and in fact with other exons being of identical sizes. Not unexpectedly, while intron positions have been conserved between paralogs their sizes have diverged considerably. Thus nearly all of the difference between the sizes of the OAT1 and 3 genes is attributable to changes in intron length.

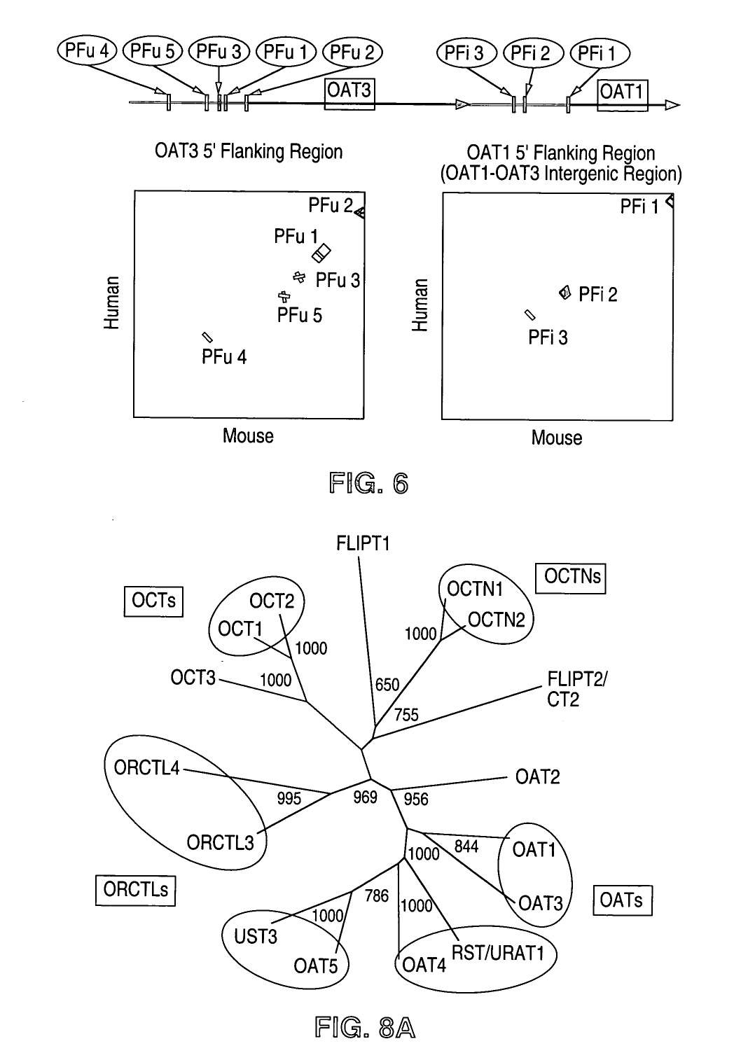

Phylogenetic footprints and potential transcription factor motifs within murine and human OAT1 and 3 were identified. The 5′ flanking sequences of murine OAT1 and 3 were compared to the orthologous human regions (derived from the draft human genome sequence) to identify PFs within the promoter regions of these genes. In the case of OAT3 the upstream 50 base pair member (FIG. 5) was compared with the 10 kb of flanking sequence and in the case of OAT1 the downstream pair member was compared with the entire intergenic sequences (7.5 kb in mouse and 8.3 kb in human). These comparisons revealed the presence of conspicuous islands of sequence conservation that clearly stood out from the large background of divergent sequence (FIG. 6) (control comparisons of the 50 flanking sequence of OAT1 to that of OAT3, i.e., comparison of paralogous rather than orthologous flanking regions did not reveal significant similarity in either species). The OAT3 5′ flanking region contains five footprints (PFu 1-5 numbered in decreasing order of significance) while the OAT1-3 intergenic region (i.e. the OAT1 5′ flanking region) contains three phylogenetic footprints (PFi 1-3 similarly numbered) (FIGS. 6 and 7). The locations of these footprints, their lengths and their percent sequence conservation are given in Table 1.

TABLE 1
Properties of the phylogenetic footprints in the 5′ flanking
sequences of the OAT1 and 3 genes

| | | | Percent conservation |
|---|---|---|---|
| Phylogenetic footprint | Location | Length (bp) | (%) |
| PFi 1 | −239 bp | 224 | 83 |
| PFi 2 | −3.7 kb | 199 | 81 |
| PFi 3 | −4.7 kb | 57 | 84 |
| PFu 1 | −2.1 kb | 420 | 81 |
| PFu 2 | −192 bp | 196 | 89 |
| PFu 3 | −2.6 kb | 113 | 74 |
| PFu 4 | −6.8 kb | 49 | 87 |
| PFu 5 | −3.5 kb | 81 | 80 |

Locations are given with respect to the transcription start site of murine OAT1 (Pfi 1-3) or AOT3 (PFu 1-5).

Over the last several years numerous transcription factor (TF) binding sites have been characterized leading to attempts to predict functional elements through identification of TF binding site matches within putative regulatory regions. However due to the degeneracy of binding sites and the large size of mammalian genomes such searches have proved highly non-specific to the extent that the great majority of computationally identified TF sites have proved non-functional. Consequently multiple strategies have been advanced to improve specificity including prioritizing sites that are clustered occur multiply or are biologically plausible. It was reasoned that only retaining TF sites that fell within PFs (using the PFs as a “filter” as it were to separate relevant from irrelevant sites) would greatly increase the likelihood of identifying functional sites. Therefore PFu 1-5 (OAT3 promoter region) was searched and PFi 1-3 (OAT1 promoter region) for TF binding site matches. Matches were retained only if present in both mouse and human sequences; i.e. TF sites were required to both occur in a generally conserved region (the PF) as well as to themselves being specifically conserved. Numerous conserved motifs were identified (boxed in FIG. 7) many of which promisingly recognize factors of demonstrated importance in the differentiation of the kidney the major site of expression of OAT1 and 3 in adult (FIG. 8B). These include PAX1 PBX WT1 (Wilms' tumor suppressor) TCF and HNF1 and are indicated by gray boxes. Among these HNF1 is a particularly plausible candidate for a role in the transcriptional regulation of OAT1 and 3 as it has been demonstrated to induce transcription of other renal transporters including the Na-phosphate cotransporter (NaPi) and the Type II Na-glucose cotransporter (SGLT2). These regulatory functions likely account in part for the finding that HNF1 knock-out mice are a model of Fanconi syndrome-proximal tubular dysfunction resulting in urinary loss of glucose amino-acids and phosphates. One might therefore predict that these knockout mice manifest defective renal excretion of organic anions.

TABLE 2
Location of the paired OAT and OCT gene in the human genome

| | Chromo- | | | Length of |
|---|---|---|---|---|
| Paired | somal | Position in | Orien- | intergenic |
| genes | band | chromosome | tation | sequence |
| OAT1 | 11q12.3 | 65,256,556-65,264,912 | Reverse | 8281 |
| OAT3 | | 65,273,192-65,294,913 | Reverse | |
| OAT5 | 11q12.3 | 65,570,112-65,584,842 | Forward | 64,885 |
| UST3 | | 65,649,726-65,690,038 | Forward | |
| OAT4 | 11q13.1 | 66,839,110-66,854,69 | Forward | 19,882 |
| URAT1 | | 66,874,571-66,885,50 | Forward | |
| OCT1 | 6q26 | 159,982,326-160,019,145 | Forward | 58,288 |
| OCT2 | | 160,077,432-160,119,328 | Reverse | |
| OCTN1 | 5q23.3 | 131,152,306-131,202,003 | Forward | 25,584 |
| OCTN2 | | 131,227,586-131,253,445 | Forward | |
| ORCTL3 | 3p22.2 | 37,596,495-37,608,97 | Forward | 27,636 |
| ORCTL4 | | 37,636,606-37,649,181 | Forward | |

Chromosomal positions are relative to the telomere of the bp arm and are based on the June 2002 assembly of the draft genome sequence.

Several conserved motifs are recognized by the myogenic factors MyoD TEF-1 and MEF2 and 3. While these factors are muscle-specific many related proteins with overlapping binding specificities are broadly expressed and thus may regulate OAT1 and 3 expression in kidney or in fetal brain/liver in which these genes are transiently expressed during development. Among the remaining motifs are a number that are bound by factors implicated in tissue-specific gene expression: POU- and homeo-domain factors (Oct-1 Brn-1 Tst-1 and S8-homeo-domains) GATA factors Isl-1, AP4, SF1, EKLF, ROAZ, and AREB6. Again while the relevance of these particular factors to OAT1 and 3 expression is not clear related proteins with overlapping binding specificities might regulate OAT1 and 3 expression through the above sites. Sites for widely expressed factors with a more general role in transcriptional regulation were noted: CREB, C/EBP, GR, CP2, and SP1. However, canonical TATA boxes in the two promoter-proximal PFs (PFi 1 and PFu 2) were not identified. Multiple OATs and OCTs are found in the genome as pairs of close paralogs. With the publication of the draft sequence of the human genome human orthologs have been identified for OAT1-5, RST/URAT1, UST3, OCT1-3, OCTN1 and 2, ORCTL3 and 4, and Flipt1 and 2; i.e. for all OATs and OCTs except UST1 and OCTN3.

An alignment of the peptide sequences of the human orthologs was performed to generate a dendrogram depicting their presumed phylogenetic relationships (FIG. 8A). As expected the dendrogram indicates that the family is broadly subdivisible into OATs, ORCTLs, OCTs, and OCTNs with the latter grouping including the somewhat divergent Flipts. Within these sub-families five pairs of closely related paralogs can be distinguished: OAT1 and 3, OAT5, and UST3, OCT1 and 2, OCTN1 and 2, and ORCTL3 and 4. In determining the locations of these genes in the human genome a remarkable feature of their chromosomal organization was noted: 12 of the 16 human orthologs occur as six tightly linked pairs; i.e. as adjoining neighbors with no interposed genes or gene predictions (Table 2; enclosed in ellipses in FIG. 8A schematically depicted in FIG. 8B). Five of these physical pairs are the same as the phylogenetic pairs noted above. The sixth pairing is of OAT4 and URAT1 which are closely related though the dendrogram suggests that OAT4 might share a more recent common ancestor with UST3 and OAT5 than it does with URAT1. The most closely linked genes are OAT1 and 3 which are separated by a scant 8.3 kb with the intergenic distances of the remaining pairs ranging from 19.9 to 64.9 kb.

The OAT1-OAT3 pair, OAT5-UST3 pair, and the OAT4-URAT1 pair occur in that order on the adjacent chromosomal bands 11q12.3 and 11q13.1 (Table 2 and FIG. 8B) with ˜280 kb separating the first and second pairs and ˜1.15 Mb separating the second and third. Of note OCT3 which is phylogenetically relatively distinct from OCT1 and 2 (FIG. 8A) is located approximately 90 kb downstream from the OCT1-2 locus. Thus, the only family members that are neither paired nor clustered with a pair (as is OCT3) are OAT2 and Flipt1 and 2.

The physical isolation of these genes mirrors their relative phylogenetic isolation as manifested by their lack of close paralogs (FIG. 8A). While several murine OATs and OCTs (including OAT4, UST3, OAT5, and ORCTL3 and 4) remain to be identified in the as yet incomplete mouse genome sequence the pairing of close paralogs appears to be preserved with murine OAT1 and 3, OCTN1 and 2, and OCT1 and 2 (mouse genome February 2002 assembly) known to exist as adjoining neighbors. Incomplete annotation of the Fugu (puffer-fish) genome precludes direct detection of OAT and OCT pairs. Nevertheless BLAST searches with various OATs and OCTs reveal multiple instances of high-scoring hits on adjacent gene predictions (e.g. gene predictions 5344 and 5345 on scaffold 401 29 716 and 29 717 on 564 and 13 752 and 13 750 on 2538 among others; Fugu genome version 3.0) suggesting that the pairing of OAT and OCT paralogs is evolutionarily ancient and not the product of recent tandem duplications.

It appears unlikely to merely reflect descent by duplication from an ancestral pair. If that were the case one would expect that the nearest phylogenetic relation of a particular pair member would be in a different pair rather than in the same pair as is the case with OATs and OCTs. However gene conversion might conceivably have increased the sequence similarity of pair members that were originally relatively divergent. Alternatively an intriguing explanation is selective pressure to hold pair members together due to the presence of shared regulatory elements such as locus control regions.

Such an explanation implies that pair members might resemble one another in their expression patterns. Pair members have similar tissue distributions. Previous studies of OAT and OCT expression suggest that pair members might indeed have similar tissue distributions. However, as these studies generally did not employ a consistent panel of tissues the degree of this similarity has been difficult to assess. Semi-quantitative PCR on the identical cDNAs was performed from 16 adult human tissues including kidney rain muscle and representative samples from the reproductive gastrointestinal and circulatory systems so as to compare the expression patterns of the paired OATs and OCTs (FIG. 8B). Possibly owing to the sensitivity of RT-PCR a faint expression of particular OATs and OCTs in tissues in which they had not been previously detected including OAT1 in liver ileum colon pancreas lung and heart OAT4 in spleen OCT1 in testis and pancreas and OCT2 in liver pancreas and heart (as well as the low expression levels in placenta testis and rain previously reported). OCTN1 and 2 and ORCTL3 and 4 are widely expressed while the other pairs are relatively restricted in expression. OAT5 and UST3 share high expression in liver and are unique among OATs and OCTs in being absent from kidney. OAT1 and 3 OAT4 and URAT1 and OCT1 and 2 are mostly expressed in kidney with significant ectopic expression of OAT1 in rain OAT4 in placenta and OCT1 in liver. Pair members do have generally similar distributions. These findings support the possibility that the physical pairing of OATs and OCTs exists to facilitate their coordinated expression. The further question arises then of what physiological advantage is conferred by such co-regulation. Coordinated expression might e expected for functionally interdependent genes (e.g. genes encoding enzymes that catalyze successive steps in a metabolic pathway). However OAT and OCT pair-members seem functionally independent both being generally expressed basolaterally and transporting overlapping groups of substrates.

Materials and Methods for OAT6 cloning and analysis. C57BL6/J mice were anesthetized with CO₂ and decapitated. Olfactory mucosa was freshly dissected, frozen in liquid nitrogen, and stored at −80° C.

Total RNA was isolated using TRI REAGENT® (Molecular Research Inc., Cincinnati Ohio) according to the manufacturer's protocol. Total RNA concentrations were quantified spectrophotometrically at 260 nm.

Tissue distribution of OAT6 was determined by RT-PCR. A panel of 7 mouse adult tissue cDNAs and 4 mouse fetal tissue cDNAs was purchased from Clonetech (Palo Alto, Calif.). Primers were designed using Primer3 (http://www-genome.wi.mit.edu) to span introns and amplify cDNA fragments of ˜400-600 bp. Primers employed for OAT6 were :forward GTTTGGGCTCAGCATCTACC (SEQ ID NO:37) and GTGGCACCAAAGGATACAGG (SEQ ID NO:38).

In addition, the expression patterns of OATs, OCTs, and OCTNs were examined by PCR on cDNA derived from olfactory epithelium. The primer sequences are as follows: OAT1 forward, TGGCTTCCTCTTTCAACTGC (SEQ ID NO:39); reverse, GGAGGCATTTCTCTGAATGG (SEQ ID NO:40). OAT2 forward, CTGGTGAGATAGGGAAAGC (SEQ ID NO:41); reverse, TAGCAGCTCCATCCTTAGGC (SEQ ID NO:42). OAT3 forward, TGCTCACTAGGCATTGTTGC (SEQ ID NO:43); reverse, TCTGTTGAGTGCTTGGATGG (SEQ ID NO:44). RST forward, TTTGGCTTCACCTTCTACGG (SEQ ID NO:45); reverse, ATCCAGGAGCCATAGACACC (SEQ ID NO:46); OCT1 forward, GAACCACTCAAGCGGTAAGG (SEQ ID NO:47); reverse, GACCATCTGCAACACAATG (SEQ ID NO:48). OCT2 forward, AGAATGGGCATCACCATAGC (SEQ ID NO:49); reverse, TCAGGGGTAAGTGAGGTTGG (SEQ ID NO:50). OCT3 forward, ATATAGTGGCAGGGGTGTCG (SEQ ID NO:51); reverse, TCCGAAATCTTTACGGTTCC (SEQ ID NO:52). OCTN1 forward, TAGCTGGGGTGCTATTCTGG (SEQ ID NO:53); reverse, TGGGGCTTTCTTCTCTGTCC (SEQ ID NO:54). OCTN2 forward, TGTCTAGGATGCACCAGAAGG (SEQ ID NO:55); reverse, TTCCCAAGCTTCTGCTAAGG (SEQ ID NO:56). OCTN3 forward, ACAACTGGTGCCTTCAGACC (SEQ ID NO:57); reverse, CCTTTAGGTTCGGAGGTTCG (SEQ ID NO:58). Control mouse G3PDH primers were provided by Clonetech. Amplifications were performed with the HotStart Taq Kit (Qiagen, Hilden, Germany). Cycle parameters were: denaturing at 95° C. for 15 min; followed by 35 cycles of 94° C. denaturing for 30 s, 60° C. annealing for 35 s, and 72° C. extension for 45 s. PCR products were visualized on 1.2% agarose gels and stained with ethidium bromide.

The Ensembl mouse genome database was searched (http://www-ensembl.org/Mus_{—}musculus/) for novel genes annotated as members of the slc22 family. Peptide sequences of OATs and OAT6 were obtained from GenBank (http://www-ncbi.nlm.nih.gov) and were aligned with ClustalX (http://www-u-strasbg.fr/BioInf/ClustalX/Top.html) with the gap penalty set to 10 and the gap extension penalty set to 0.1.Dendrograms were generated from ClustalX output using TreeView (http:-//taxonomy.zoology.gla.ac.uk/). Topologies for OAT6 were predicted using TopPred (http:-//bioweb.pasteur.fr/seqan1/interfaces/topred.html) using default parameters. The SOURCE database (http:-//source.stanford.edu) was used as database for mouse ESTs.

A number of embodiments of the invention have been described. Nevertheless, it will be understood that various modifications may be made without departing from the spirit and scope of the invention. Accordingly, other embodiments are within the scope of the following claims.

1. A double knockout non-human transgenic animal that lacks expression of at least two slc22 family member—organic ion transporters.

2. The double knockout non-human transgenic animal of claim 1, wherein the two organic ion transporters are both basolateral organic ion transporters.

3. The double knockout non-human transgenic animal of claim 1, wherein the transgenic knockout is 6AT3^{-/-}.

4. The double knockout non-human transgenic animal of claim 2, wherein the transgenic knockout is OAT1^{-/-} and OAT3^{-/-}.

5. The double knockout non-human transgenic animal of claim 1, wherein the two organic ion transporters are both apical organic ion transporters.

6. The double knockout non-human transgenic animal of claim 5, wherein the knockout lacks OAT4 and RST.

7. The double knockout non-human transgenic animal of claim 1, wherein the knockout lacks expression of at least two genes selected from the group consisting OAT1, OAT3, OAT4 and RST.

8. The double knockout non-human transgenic animal of claim 1, wherein the two organic ion transporters comprise one basolateral organic ion transporter and one apical organic ion transporter.

9. A non-human mammal that carries germline mutations in at least two slc22 family member—organic ion transport genes.

10. The mammal of claim 9 which is a mouse.

11. A method for producing the double knockout non-human mammal of claim 9 comprising the steps of:
(i) providing an embryonic stem (ES) cell from the relevant animal species comprising a first intact OAT gene;
(ii) providing a first targeting vector capable of disrupting the first intact OAT gene;
(iii) introducing the first targeting vector into the ES cells under conditions where the intact first OAT undergoes homologous recombination with the first targeting vector to produce a mutant first OAT gene;
(iv) introducing the ES cells carrying a disrupted first OAT gene into a blastocyst;
(v) implanting the blastocyst into the uterus of pseudopregnant female;
(vi) delivering animals from said females, identifying a first mutant animal that carries the mutant allele and obtaining mutant ES cells from the first mutant animal;
(v) providing a second targeting vector capable of disrupting a second intact OAT gene;
(vi) introducing the second targeting vector into the mutant ES cells under conditions where the intact second OAT gene undergoes homologous recombination with the second targeting vector to produce a mutant second OAT gene;
(vii) introducing the mutant ES cells carrying a disrupted second OAT gene into a blastocyst;
(viii) implanting the blastocyst into the uterus of pseudopregnant female;
(ix) delivering animals from said females; and
(x) selecting for OAT double knockout animals and breeding them.

12. The method of claim 11, wherein the mammal is a mouse.

13. The use of the double knockout mammal of claim 9, as a model for drug toxicity studies.

14. The use of claim 13, wherein the mammal is a mouse.

15. A method for determining whether a compound has toxic effects in humans, comprising administering the compound to an slc22 family member—organic ion transporter double knockout non-human mammal and evaluating any toxicity in the knockout non-human mammal.

16. A cell line derived from a double knockout animal of claim 9.

17. The cell line of claim 16, transfected with a wild type or modified organic ion transporter polynucleotide.

18. A cell of claim 16, wherein the cell is selected from the group consisting of stem cells, epithelial cells and renal cells, and blood brain barrier cells.

19. A method of determine a drug treatment for a mammalian subject comprising:
(i) identifying a polymorphism in an slc22 family member—organic ion transporter;
(ii) determining if the polymorphism is associated with drug sensitivity using a knockin of an slc22 family member—organic ion transporter gene containing a polymorphism in a non-human transgenic animal; and
(iii) identifying a drug that is efficacious for the subject based upon the polymorphism and any association with drug sensitivity based upon the polymorphism.

20. The method of claim 6, wherein the polymorphism is in a gene selected from OAT1, OAT3, OAT4, OAT6 and RST.

21. A substantially purified polypeptide selected from the group consisting of:
(a) a polypeptide comprising SEQ ID NO:36;
(b) a polypeptide encoded by a polynucleotide comprising SEQ ID NO:35;
(c) a polypeptide comprising a sequence that is at least 80% identical to SEQ ID NO:36 and has OAT6 activity;
(d) a polypeptide that is encoded by a polynucleotide that hybridizes to a nucleic acid consisting of SEQ ID NO:35 under moderate to high stringency conditions and wherein the polypeptide has OAT6 activity; and
(e) a polypeptide comprising a fragment of any of (a) to (d) above having OAT6 activity.

22. The substantially purified polypeptide of claim 21, wherein the polypeptide consists of SEQ ID NO:36.

23. The substantially purified polypeptide of claim 21, wherein the fragment is a soluble domain of a polypeptide consisting of SEQ ID NO:36.

24. The substantially purified polypeptide of claim 21 fused to a second polypeptide moiety.

25. An isolated polynucleotide encoding the polypeptide of claim 21.

26. An isolated polynucleotide selected from the group consisting of:
(a) a polynucleotide comprising SEQ ID NO:35;
(b) a polynucleotide that encodes a polypeptide having a sequence as set forth in SEQ ID NO:36;
(c) a polynucleotide that hybridizes to the complement of a nucleic acid consisting of SEQ ID NO:35, under stringent conditions of 0.5 M NaHPO₄, 7% sodium dodecyl sulfate (SDS), 1 mM EDTA at 65° C., and washing in 0.1×SSC/0.1% SDS at 68° C. and encodes a functionally equivalent OAT6 polypeptide;
(d) a polynucleotide that hybridizes to the complement of a nucleic acid consisting of SEQ ID NO:35, under moderately stringent conditions of washing in 0.2% SSC/0.2% SDS/0.1% SDS at 42° C. and encodes a functionally equivalent OAT6 polypeptide; and
(e) a fragment of any of (a) to (d) that are at least 15 nucleotides in length.

27. A vector comprising an isolated polynucleotide of claim 26.

28. A recombinant host cell comprising an isolated polynucleotide of claim 26.

29. A recombinant host cell comprising the vector of claim 27.

30. A transgenic organism comprising a knockout of OAT6.


SEQUENCE LISTING (85 sequences)

SEQ ID NO: 1 - DNA, 24 bp, Artificial sequence
  primer
cagtcttcatggcaggtatactgg

SEQ ID NO: 2 - DNA, 23 bp, Artificial sequence
  primer
gcgcatgctccagactgccttgg

SEQ ID NO: 3 - DNA, 20 bp, Artificial sequence
  primer
gtgtagcgccaagtgccagc

SEQ ID NO: 4 - DNA, 24 bp, Artificial sequence
  primer
gacaaagagaaggctatgacctgg

SEQ ID NO: 5 - DNA, 20 bp, Artificial Sequence
  primer
atgcctatccacacccgtgc

SEQ ID NO: 6 - DNA, 20 bp, Artificial sequence
  primer
ggcaaagctagtggcaaacc

SEQ ID NO: 7 - DNA, 22 bp, Artificial sequence
  primer
gctgcatgatggtgtggtttgg

SEQ ID NO: 8 - DNA, 22 bp, Artificial sequence
  primer
gtacaactcggacgtgaacagg

SEQ ID NO: 9 - DNA, 24 bp, Artificial sequence
  primer
cagtcttcatggcaggtatactgg

SEQ ID NO: 10 - DNA, 20 bp, Artificial sequence
  primer
ctgtagccagcgccactgag

SEQ ID NO: 11 - DNA, 22 bp, Artificial sequence
  primer
gaaggagccaaattgagtatgg

SEQ ID NO: 12 - DNA, 22 bp, Artificial sequence
  primer
tacaggaagatgcagttgaagg

SEQ ID NO: 13 - DNA, 22 bp, Artificial sequence
  primer
ctatgggtgtggaagaatttgg

SEQ ID NO: 14 - DNA, 22 bp, Artificial sequence
  primer
tcccgtaaagatgatattgggg

SEQ ID NO: 15 - DNA, 20 bp, Artificial sequence
  primer
ctgggaaagggatgttttgg

SEQ ID NO: 16 - DNA, 20 bp, Artificial sequence
  primer
accgtctcgttattggttgg

SEQ ID NO: 17 - DNA, 20 bp, Artificial sequence
  primer
ctttggcttcaccttcttcg

SEQ ID NO: 18 - DNA, 22 bp, Artificial sequence
  primer
gtggattttaggacagagttcc

SEQ ID NO: 19 - DNA, 20 bp, Artificial sequence
  primer
accagaggaaggcttaaagg

SEQ ID NO: 20 - DNA, 21 bp, Artificial sequence
  primer
ggtggagaatacacacttagg

SEQ ID NO: 21 - DNA, 20 bp, Artificial sequence
  primer
tgtaagatccaccatgcagg

SEQ ID NO: 22 - DNA, 20 bp, Artificial sequence
  primer
ccgttaaggtcatcaagagg

SEQ ID NO: 23 - DNA, 20 bp, Artificial sequence
  primer
gaacctctacctggatttcc

SEQ ID NO: 24 - DNA, 20 bp, Artificial sequence
  primer
ttcatggtctctggcaaagc

SEQ ID NO: 25 - DNA, 20 bp, Artificial sequence
  primer
ctcatgcttgggaagaatgg

SEQ ID NO: 26 - DNA, 20 bp, Artificial sequence
  primer
ctcatgcttgggaagaatgg

SEQ ID NO: 27 - DNA, 21 bp, Artificial sequence
  primer
gttactttgctctgtctctgg

SEQ ID NO: 28 - DNA, 21 bp, Artificial sequence
  primer
catctgctctaaggtttctgg

SEQ ID NO: 29 - DNA, 20 bp, Artificial sequence
  primer
ttgacctgtgtctgacttgc

SEQ ID NO: 30 - DNA, 22 bp, Artificial sequence
  primer
ggagcatttattatgagcctgg

SEQ ID NO: 31 - DNA, 20 bp, Artificial sequence
  primer
aagaccagccttgcttatgg

SEQ ID NO: 32 - DNA, 20 bp, Artificial sequence
  primer
tatgacccagtgacctatgg

SEQ ID NO: 33 - DNA, 20 bp, Artificial sequence
  primer
cagagctgaaatccatgacg

SEQ ID NO: 34 - DNA, 21 bp, Artificial sequence
  primer
catacttggccactcaattcc

SEQ ID NO: 35 - DNA, 1671 bp, Artificial sequence
  cDNA
atggccttcacagacctgctggatgccctgggaggcgtgggtcgcttccagcttgtctatacagccttgctgctgctgccctgtgggctgctggcttgccataccttcctacagaacttcacggctgctgcaccccctcaccactgccaacatcctgccaactacacagaacctaccaccaatgtctcaggggtctggctgagggctgccatacccctgaaccagcatggggaccccgagccgtgccggcgctatgtagaacctcagtgggcccttctgaaacccaacgcctcctcccatggggtggccaccgagggctgcaaggatggttgggtctacgaccgaagcattttcccatctaccattgtgatggagtgggacctggtgtgtgaggcccgcacccttcgtgacctggctcagtccatctacatgtccggggtgttggtgggagccgccttgtttggtggtcttgctgacaggttgggtcgcaaggccccactggtgtggtcgtacctgcagctggcagtttcgggggctgccacagcgtatgtgggctccttcagtgcctactgcgtcttccgcttcctgatgggcatgaccttctccggcatcatccttaactccctctccctggttgtagaatggatgccaaccagaggccgtacagtagcaggcatcttgttgggtttctccttcaccttgggccagctcatcctggctggcgtggcatacctgatccggccctggcggtggctgcagtttgctgtgtctgctccttttctggtctttttcctctattcttggtggcttccagagtcatcccgatggctcctccttcacggcaaggcccagcaagctgtgcagaaccttcagaaggtggccatgatgaatggcaggaaggcggaaggggagaggctgactacagaggtggtgagctcctacatccaggatgagtttgcaagtgtccgcacctccaactccatcttggacctctttagaacccctgccatcagaagggtcacgtgctgtctcatgggggtctggttctctaattctgtggcttactacggcctggccatggacctgcagaagtttgggctcagcatctacctggtacaggctctgtttgggatcatcgatatcccggccatgctggtcgccaccaccacgatgatttatgtgggacgacgggccacagtgtcctccttcctcatactagctgggctcatggtcattgccaacatgtttatgcctgaggatctgcagaccttgcggacagtgcaggcagcgctgggcaaaggctgcctggccagctccttcatctgcgtgtatctgttcacgggagaactctatcccacagagatcaggcagatggggatgggatttgcttctgtcaatgcccgcctcggaggcttggtggcacccctgatcaccacccttggcgagatcagtccagttctgccacctgtatcctttggtgccacttcagtcttggctggaatggctgtcgcctgcttcctgactgagacccgcaatgtgccactggtagaaactattgctgcaatggagagacgagtcaaacaaggccgttccaaaagagacacagaacagaagagtgaagaaatttctcttcagcagctgggggcttcacccctcaaagaaaccatttaa

SEQ ID NO: 36 - protein, 556 aa, Artificial sequence
  predicted amino acid sequence
MAFTDLLDALGGVGRFQLVYTALLLLPCGLLACHTFLQNFTAAAPPHHCQHPANYTEPTTNVSGVWLRAAIPLNQHGDPEPCRRYVEPQWALLKPNASSHGVATEGCKDGWVYDRSIFPSTIVMEWDLVCEARTLRDLAQSIYMSGVLVGAALFGGLADRLGRKAPLVWSYLQLAVSGAATAYVGSFSAYCVFRFLMGMTFSGIILNSLSLVVEWMPTRGRTVAGILLGFSFTLGQLILAGVAYLIRPWRWLQFAVSAPFLVFFLYSWWLPESSRWLLLHGKAQQAVQNLQKVAMMNGRKAEGERLTTEVVSSYIQDEFASVRTSNSILDLFRTPAIRRVTCCLMGVWFSNSVAYYGLAMDLQKFGLSIYLVQALFGIIDIPAMLVATTTMIYVGRRATVSSFLILAGLMVIANMFMPEDLQTLRTVQAALGKGCLASSFICVYLFTGELYPTEIRQMGMGFASVNARLGGLVAPLITTLGEISPVLPPVSFGATSVLAGMAVACFLTETRNVPLVETIAAMERRVKQGRSKRDTEQKSEEISLQQLGASPLKETI

SEQ ID NO: 37 - DNA, 20 bp, Artificial sequence
  primer
gtttgggctcagcatctacc

SEQ ID NO: 38 - DNA, 20 bp, Artificial sequence
  primer
gtggcaccaaaggatacagg

SEQ ID NO: 39 - DNA, 20 bp, Artificial sequence
  primer
tggcttcctctttcaactgc

SEQ ID NO: 40 - DNA, 20 bp, Artificial sequence
  primer
ggaggcatttctctgaatgg

SEQ ID NO: 41 - DNA, 19 bp, Artificial sequence
  primer
ctggtgagatagggaaagc

SEQ ID NO: 42 - DNA, 20 bp, Artificial sequence
  primer
tagcagctccatccttaggc

SEQ ID NO: 43 - DNA, 20 bp, Artificial sequence
  primer
tgctcactaggcattgttgc

SEQ ID NO: 44 - DNA, 20 bp, Artificial sequence
  primer
tctgttgagtgcttggatgg

SEQ ID NO: 45 - DNA, 20 bp, Artificial sequence
  primer
tttggcttcaccttctacgg

SEQ ID NO: 46 - DNA, 20 bp, Artificial sequence
  primer
atccaggagccatagacacc

SEQ ID NO: 47 - DNA, 20 bp, Artificial sequence
  primer
gaaccactcaagcggtaagg

SEQ ID NO: 48 - DNA, 19 bp, Artificial sequence
  primer
gaccatctgcaacacaatg

SEQ ID NO: 49 - DNA, 20 bp, Artificial sequence
  primer
agaatgggcatcaccatagc

SEQ ID NO: 50 - DNA, 20 bp, Artificial sequence
  primer
tcaggggtaagtgaggttgg

SEQ ID NO: 51 - DNA, 20 bp, Artificial sequence
  primer
atatagtggcaggggtgtcg

SEQ ID NO: 52 - DNA, 20 bp, Artificial sequence
  primer
tccgaaatctttacggttcc

SEQ ID NO: 53 - DNA, 20 bp, Artificial sequence
  primer
tagctggggtgctattctgg

SEQ ID NO: 54 - DNA, 20 bp, Artificial sequence
  primer
tggggctttcttctctgtcc

SEQ ID NO: 55 - DNA, 21 bp, Artificial sequence
  primer
tgtctaggatgcaccagaagg

SEQ ID NO: 56 - DNA, 20 bp, Artificial sequence
  primer
ttcccaagcttctgctaagg

SEQ ID NO: 57 - DNA, 20 bp, Artificial sequence
  primer
acaactggtgccttcagacc

SEQ ID NO: 58 - DNA, 20 bp, Artificial sequence
  primer
cctttaggttcggaggttcg

SEQ ID NO: 59 - DNA, 415 bp, mouse
gggggcgctgcccggatacagcggcccctcactgagctgttgggctatgtatactctggctcaaccccagaagcctcagggaaatcaccaggcgcctttaaacaacatcccactgggacaggaacagacggacagcccaggccaattaggaactagaaaagaacatctctgtgagggtgaacacccagaacagggggttagctgcagcagcccagcttgctgcaagcctcccagtcaaaaccttcctaaaacaaccctttggcggccttggagtataatttaatcttcctttgatgactccagatttcaccctgcctcaggtcactcggagaacatcaataattgctgagtccaggtggtaagcgaagaggaggggctggaacagggtttgtcatctgaccttgctttgtggtag

SEQ ID NO: 60 - DNA, 415 bp, Homo sapien
gggggcgctgcccggatgcagctgcccctggctgggcagtccagatatgtatactctggcttaactccagcagcctcagcggggagccaccaggctcctttaaacaacattccactgggaaaggaacagatggataacccaagccaattaggaaccagaaaatctccaagaggccaaatgcccagaacagggggagctcagggcggcagcccagctcactgcaagcctcacagtcgaaaatcttcctaaaacaaccccttggctgccttggagcataatttaatcttcctttgatgactccagatctcgccctgcctcaggccactctgagaacatcaataactgctgggcccaggtgggaagccgaggggcagggctggacccaggttgaccagctgaccctgccctgcggtag

SEQ ID NO: 61 - DNA, 196 bp, mouse
gctgaggcagccctttcaggagagctgggcttggtgggtgcacagcattccccctgccggatgttaatcttccaaagaaagtcaaacattagcccaggaaacagcttatgccttatataaggcaccccaggggagacacaaacagcttgttagagctgagctgtcctactacagcagctgctggaccctaggacag

SEQ ID NO: 62 - DNA, 200 bp, Homo sapien
gctgaggcagccctttgaggagagctgggcttggtgggtccacagcactctccctgccagtgacgttaatccgcaaaagaaagtcaaacattagcccaggaaacagcttatgccttatataagcccccctgggggaggcacaaacacagcttgttagagctgagctgccctactacagcagctgccggcccctaggacag

SEQ ID NO: 63 - DNA, 113 bp, mouse
  misc_feature (67)..(78)
  misc_feature (67)..(78): n is a, c, g, or t
ctatttccatgtcccaaattaaaaatacattgaatgtgtaagatttcttaagtacataatttctttnnnnnnnnnnnngtcaatttgaacacattaaatttaggatcacagca

SEQ ID NO: 64 - DNA, 112 bp, Homo sapien
ctatttccatatcccaaattaaaaatgcatctagcatgtaagatttcccaaatttatagtgtctttaaaaaaaaaaagttaatttgaacatattgaatttaggatcacagca

SEQ ID NO: 65 - DNA, 49 bp, mouse
cagagaaggcaatggaattccagaaggtaggaggaagggtcgcctacac

SEQ ID NO: 66 - DNA, 49 bp, Homo sapien
cagaaaagacaaagggattccagaaggtgggaggaggggtcgcctacac

SEQ ID NO: 67 - DNA, 80 bp, mouse
aggacagaaaagcctaggggctcaccagatcctctgagactgtcacagagacatcctggccctgaggccaaggccaccac

SEQ ID NO: 68 - DNA, 81 bp, Homo sapien
aggacagaagagcccaggggattatcggcccctctgagactatcgcagagatgtcccagcccctgagaccaaggccaccac

SEQ ID NO: 69 - DNA, 223 bp, mouse
tccatcctcccttgcccttcattcccaattggagaaattccactgacacaaggaatactcacagggttaatccttctgacaccaagtcacactttaactcattgtcatcaggacaaagattaaacgctgcctgcgtaagagtcagggctccagcagaccctgaaagctgagctgtccagacccccgaagtgaagaaaagaggcgagggcaagggagggccaga

SEQ ID NO: 70 - DNA, 224 bp, Homo sapien
tccaccctctcctgccctttataaccacttggagaaattccactgacacaaggaatccttggagggttaatccttctgataccaagtcacactttaactcattccctccaggccaaggattaaaaactgcccatgcaagggtcaggtctccagcagaccctgaaagctgagctgcccatacccccaaagtgaggagaagctgcaagggaaaagggagggacaga

SEQ ID NO: 71 - DNA, 196 bp, mouse
cctcagcactccacctactcctctagaagagcactaagtctcggcagggaactgaccccacaacctctaagctgtttgtccagctgtcctcgcctccccaggccacaggttcttatataccttggcctggagaatgtatcaggcatctggacacccatggagcccaagctagaatcaacacatgctgggaggggga

SEQ ID NO: 72 - DNA, 199 bp, Homo sapien
cctcagcactccacccactcccccatgagggcaccaggtcctggcaggagactggccccgcagtccccaagctgtttgtccagctgtcctcgcatccccagaccacaggactcttatgtatcttggcctggagaatgttgtcaggcatctggacacccactgggtcccagcccagaaccaacaacgggtgggaggggga

SEQ ID NO: 73 - DNA, 57 bp, mouse
agtgtcttctttgatctgtaaattatttatttgaaaggtgtcacaaagtaataaaaa

SEQ ID NO: 74 - DNA, 57 bp, Homo sapien
agtgttttatatgatctgtaaattatgtatttgaaaaatctcacaaaagaataaaaa

SEQ ID NO: 75 - protein, 556 aa, Unknown
  transmembrane protein
MPTVDDVLEHVGEFGWFQKQAFLLLCLISASLAPIYVGIVFLGFTPDHHCRSPGVAELSQRCGWSPAEELNYTVPGLGSAGEASFLSQCMKYEVDWNQSTLDCVDPLSSLAANRSHLPLSPCEHGWVYDTPGSSIVTEFNLVCGDAWKVDLFQSCVNLGFFLGSLVVGYIADRFGRKLCLLVTTLVTSLSGVLTAVAPDYTSMLLFRLLQGMVSKGSWVSGYTLITEFVGSGYRRTTAILYQVAFTVGLVGLAGVAYAIPDWRWLQLAVSLPTFLFLLYYWFVPESPRWLLSQKRTTQAVRIMEQIAQKNRKVPPADLKMMCLEEDASERRSPSFADLFRTPSLRKHTLILMYLWFSCAVLYQGLIMHVGATGANLYLDFFYSSLVEFPAAFIILVTIDRIGRIYPIAASNLVAGAACLLMIFIPHELHWLNVTLACLGRMGATIVLQMVCLVNAELYPTFIRNLGMMVCSALCDLGGIFTPFMVFRLMEVWQALPLILFGVLGLSAGAVTLLLPETKGVALPETIEEAENLGRRKSKAKENTIYLQVQTGKSPHT

SEQ ID NO: 76 - protein, 553 aa, Unknown
  transmembrane protein
MPTVDDILEHIGEFHLFQKQTFFLLALLSGAFTPIYVGIVFLGFTPNHHCRSPGVAELSQRCGWSPAEELNYTVPGLGSAGEVSFLSQCMRYEVDWNQSTLDCVDPLSSLAANRSHLPLSPCEHGWVYDTPGSSIVTEFNLVCAHSWMLDLFQSLVNVGFFIGAVGIGYLADRFGRKFCLLVTILINAISGVLMAISPNYAWMLVFRFLQGLVSKAGWLIGYILITEFVGLGYRRTVGICYQIAFTVGLLILAGVAYALPNWRWLQFAVTLPNFCFLLYFWCIPESPRWLISQNKNAKAMKIIKHIAKKNGKSVPVSLQSLTADEDTGMKLNPSFLDLVRTPQIRKHTLILMYNWFTSSVLYQGLIMHMGLAGDNIYLDFFYSALVEFPAAFIIILTIDRIGRRYPWAVSNMVAGAACLASVFIPDDLQWLKITVACLGRMGITIAYEMVCLVNAELYPTYIRNLAVLVCSSMCDIGGIVTPFLVYRLTDIWLEFPLVVFAVVGLVAGGLVLLLPETKGKALPETIEDAEKMQRPRKKKEKRIYLQVKKAELS

SEQ ID NO: 77 - protein, 551 aa, Unknown
  transmembrane protein
MPTFDQALRKAGEFGRFQRRVFLLLCLTGVTFAFLFVGVVFLGSQPDYYWCRGPRATALAERCAWSPEEEWNLTTPELHVPAERRGQGHCHRYLLEATNTSSELSCDPLTAFPNRSAPLVSCSGDWRYVETHSTIVSQFDLVCSNAWMLDLTQAILNLGFLAGAFTLGYAADRYGRLIIYLISCFGVGITGVVVAFAPNFSVFVIFRFLQGVFGKGAWMTCFVIVTEIVGSKQRRIVGIVIQMFFTLGIIILPGIAYFTPSWQGIQLAISLPSFLFLLYYWVVPESPRWLITRKQGEKALQILRRVAKCNGKHLSSNYSEITVTDEEVSNPSCLDLVRTPQMRKCTLILMFAWFTSAVVYQGLVMRLGLIGGNLYIDFFISGLVELPGALLILLTIERLGRRLPFAASNIVAGVSCLVTAFLPEGIPWLRTTVATLGRLGITMAFEIVYLVNSELYPTTLRNFGVSLCSGLCDFGGIIAPFLLFRLAAIWLELPLIIFGILASVCGGLVMLLPETKGIALPETVEDVEKLGSSQLHQCGRKKKTQVSTSDV

SEQ ID NO: 78 - protein, 545 aa, Unknown
  transmembrane protein
MAFNDLLKQVGGVGRFQLIQVTMVVAPLLLMASHNTLQNFTAAIPAHHCRPPANANLSKDGGLEAWLPLDKQGRPESCLRFPFPHNGTEANGTGVTEPCLDGWVYDNSTFPSTIVTEWNLVCSHRAFRQLAQSLFMVGVLLGAMMFGYLADRLGRRKVLILNYLQTAVSGTCAAYAPNYTVYCIFRLLSGMSLASIAINCMTLNMEWMPIHTRAYVGTLIGYVYSLGQFLLAGIAYAVPHWRHLQLAVSVPFFVAFIYSWFFIESARWYSSSGRLDLTLRALQRVARINGKQEEGAKLSIEVLQTSLQKELTLNKGQASAMELLRCPTLRRLFLCLSMLWFATSFAYYGLVMDLQGFGVSMYLIQVIFGAVDLPAKFVCFLVINSMGRRPAQLASLLLAGICILVNGIIPRGHTIIRTSLAVLGKGCLASSFNCIFLYTGELYPTMIRQTGLGMGSTMARVGSIVSPLISMTAEFYPSIPLFIFGAVPVAASAVTALLPETLGQPLPDTVQDLKSRSRGKQKQQQLEQQKQMIPLQVSTQEKNGL

SEQ ID NO: 79 - protein, 537 aa, Unknown
  transmembrane protein
MTFSEILDRVGSMGPFQYLHVTLLALPILGIANHNLLQIFTATTPDHHCRPPPNASLEPWVLPLGPNGKPEKCLRFVHLPNASLPNDTQGATEPCLDGWIYNSTRDTIVTEWDLVCGSNKLKEMAQSVFMAGILVGGPVFGELSDRFGRKPILTWSYLLLAASGSSAAFSPSLTVYMIFRFLCGCSISGISLSTIILNVEWVPTSTRAISSTTIGYCYTIGQFILPGLAYAVPQWRWLQLSVSAAFFIFSLLSWWVPESIRWLVLSGKFSKALKTLQRVATFNGKKEEGEKLTVEELKFNLQKDITSAKVKYGLSDLFRVSILRRVTFCLSLAWFATGFAYYSLAMGVEEFGVNIYILQIIFGGVDIPAKFITILSISYLGRRITQGFLLILAGVAILALIFVSSEMQLLRTALAVFGKGCLSGSFSCLFLYTSELYPTVLRQTGMGISNIWARVGSMIAPLVKITGELQPFIPNVIFVTMTLLGGSAAFFLLETLNRPLPETIEDIQDWYQQTKKTKQEPEAEKASQTIPLKTGGP

SEQ ID NO: 80 - protein, 556 aa, Unknown; identical to SEQ ID NO: 36
  transmembrane protein

SEQ ID NO: 81 - protein, 553 aa, Unknown
  transmembrane protein
MAFPELLDRVGGLGRFQLFQTVALVTPILWVTTQNMLENFSAAVPHHRCWVPLLDNSTSQASIPGDLGPDVLLAVSIPPGPDQQPHQCLRFRQPQWQLTESNATATNWSDAATEPCEDGWVYDHSTFRSTIVTTWDLVCNSQALRPMAQSIFLAGILVGAAVCGHASDRFGRRRVLTWSYLLVSVSGTAAAFMPTFPLYCLFRFLLASAVAGVMMNTASLLMEWTSAQGSPLVMTLNALGFSFGQVLTGSVAYGVRSWRMLQLAVSAPFFLFFVYSWWLPESARWLITVGKLDQGLQELQRVAAVNRRKAEGDTLTMEVLRSAMEEEPSRDKAGASLGTLLHTPGLRHRTIISMLCWFAFGFTFYGLALDLQALGSNIFLLQALIGIVDFPVKTGSLLLISRLGRRFCQVSFLVLPGLCILSNILVPHGMGVLRSALAVLGLGCLGGAFTCITIFSSELFPTVIRMTAVGLCQVAARGGAMLGPLVRLLGVYGSWMPLLVYGVVPVLSGLAALLLPETKNLPLPDTIQDIQKQSVKKVTHDTPDGSILMSTRL

SEQ ID NO: 82 - protein, 557 aa, Unknown
  transmembrane protein
MRDYDEVTAFLGEWGPFQRLIFFLLSASIIPNGFNGMSIVFLAGTPEHRCLVPHTVNLSSAWRNHSIPLETKDGRQVPQKCRRYRLATIANFSELGLEPGRDVDLEQLEQESCLDGWEYDKDVFLSTIVTEWDLVCKDDWKAPLTTSLFFVGVLMGSFISGQLSDRFGRKNVLFLTMGMQTGFSFLQVFSVNFEMFTVLFVLVGMGQISNYVAAFVLGTEILSKSIRIIFATLGVCIFYAFGFMVLPLFAYFIRDWRMLLLALTVPGVLCGALWWFIPESPRWLISQGRIKEAEVIIRKAAKINGIVAPSTIFDPSELQDLNSTKPQLHHIYDLIRTRNIRVITIMSIILWLTISVGYFGLSLDTPNLHGDIYVNCFLLAAVEVPAYVLAWLLLQYLPRRYSISAALFLGGSVLLFMQLVPSELFYLSTALVMVGKFGITSAYSMVYVYTAELYPTVVRNMGVGVSSTASRLGSILSPYFVYLGAYDRFLPYILMGSLTILTAILTLFFPESFGVPLPDTIDQMLRVKGIKQWQIQSQTRMQKDGEESPTVLKSTAF

SEQ ID NO: 83 - protein, 564 aa, Unknown
  transmembrane protein
MLDYDEVTAFLGEWGTFQRLIFFLLSASIIPNGFTGLSAVFLTAIPEHRCRIPDTVNLSSAWRNHSIPMETKDGPEVPQKCRRYRLATIANFSELGLEPGRDVDLEQLEQENCLDGWEYDKDIFLSTIVTEWDLVCKDDWKAPLTTSFFYVGVLLGSFISGQLSDRFGRKNILFLTMAMHTGFSFIQVFSVNFEMFTLLYTLVGMGHISNYVAAFVLGTEMLSKSVRIIFATLGVCIFFAFGFMVLPLFAYFIREWRRLLLAITLPGVLCGALWWFIPESPRWLISQGRIKEAEVIIRKAAKINGIVAPSTIFDPSETNKLQDDSSKKPQSHHIYDLVRTPNIRILTIMSIILWLTISVGYFGLSLDTPNLNGNIYVNCFLLAAVEVPAYVLAWLLLQHVSRRYSMAGSLFLGGSVLLLVQLVPSDLHYLSTTLVMVGKFGITSAYSMVYVYTAELYPTVVRNMGVGVSSTASRLGSILSPYFVYLGAYDRRLPYILMGSLTILTAIITLFFPESSGVSLPETIDEMQKVKKLKQRQSLSKKGSPKESKGNVSRTSRTSEPKGF

SEQ ID NO: 84 - protein, 551 aa, Unknown
  transmembrane
MAFQDLIIQIGSLGRFQILHMIFVLICHALSAPHTLLENFTAAIPSHRCWVPILDNDTASDNGSRILSQDDLLRISIPLDSNLRPDKCRRYIQPQWHLLHLNGTFPTVTEPDTEPCVDGWVYDQSTFLSTTVTQWDLVCGSQALNSVAKFIYMTGIFIGYIMGGHLSDKFGRKLIFTCSLLKMAITETCVAFAPSFLIYCSLRFLSGIFSSTLRTNSALLILEWTSPKFQALVMALIFIASGVGQTLLGGLAFAFRNWHHLQLALSVPMFLLLIPTRWLSESARWLIMANKPQKSLKELKKAACVNRIKNSGDALTLEVVKTIMKEELEAAQTKPSPLDLFRTPNLRKRICLLSFVRFVSVMSLLGLLINIQYLSNNVFLLQCLYGVVCIPANVLGNFSMNYMGRRMTQLIFMSVLGISILAVVFLPQEMQILRVFLSTLGGAISSASITSTLVHANELVPTIIRATALGVVGIAGSAGGALSPLLMILTTYSASLPWIIYGILPFLGGLVALLLPETKNQPLPDSIQDIENKRKSSKEAKKDVVAKVTPL

SEQ ID NO: 85 - protein, 540 aa, Unknown
  transmembrane protein
MGFEELLHKVGGFGPFQLRNLVLLALPRFLLPMHFLLPIFMAAVPAHHCALPDAPANLSHQDLWLKTHLPRETDGSFSSCLRFAYPQALPNVTLGTEVYNSGEPEGEPLTVPCSQGWEYDRSEFSSTIATEWDLVCEQRGLNKVTSTCFFIGVLLGAVVYGYLSDRFGRRRLLLVAYVSTLALGLMSAASVNYIMFVTTRMLTGSALAGFTIIVLPLELEWLDVEHRTVAGVISTTFWTGGVLLLTLVGYLIRSWRWLLLAATLPCVPGIISIWWVPESARWLLTQGRVEEAKKYLSICAKLNGRPISEDSLSQEALNKVITMERVSQRPSYLDLFRTSQLRHVSLCCMMMWFGVNFSYYGLTLDASGLGLTVYQTQLLFGAVEVPSKITVFFLVRLVGRRLTEAGMLLATALTFGISLLVSSDTKSWITALVVIGKAFSEAAFTTAYLFTSELYPTVLRQTGMGFTALIGRLGASLAPLVVLLDGVWLLLPKLAYGGISFLAACTVLLLPETKKAQLPETIQDVERKGRKIDRSGTELA